(19) United States
(12) Patent Application Publication
(10) Pub. No.: US 2019/0195891 A1
(43) Pub. Date: Jun. 27, 2019

(54) DIAGNOSTIC AND THERAPEUTIC USES OF EXOSOMES

(71) Applicant: Yeda Research and Development Co. Ltd., Rehovot (IL)

(72) Inventors: David WALLACH, Rechovot (IL); Andrew KOVALENKO, Rehovot (IL); Seongmin YOON, Rehovot (IL)

(73) Assignee: Yeda Research and Development Co. Ltd., Rehovot (IL)

(21) Appl. No.: 16/325,393

(22) PCT Filed: Aug. 18, 2017
(86) PCT No.: PCT/IL2017/050922

§ 371 Date: Feb. 14, 2019

(30) Foreign Application Priority Data
Aug. 18, 2016 (IL) 247368

(51) Int. Cl. G01N 33/68; C12Q 1/48

(52) U.S. Cl. CPC G01N 33/6893; G01N 2800/7095; C12Q 1/485

(57) ABSTRACT

A method of detecting activation of a necroptosis activation pathway in a subject is disclosed. The method comprising: (a) obtaining a biological sample comprising exosomes from the subject; (b) detecting an activity or expression of a component of the necroptosis activation pathway in an exosome fraction of the biological sample, wherein an increase in the activity or expression of the component of the necroptosis activation pathway indicates the activation of said necroptosis activation pathway. Methods of diagnosing necroptosis or inflammation by determining the level of exosomes in a biological sample. Method of modulating endocytosis by inhibiting MLKL or a cell surface receptor and a pharmaceutical composition comprising a population of exosomes comprising a component of the necroptosis activation pathway and its use in therapy of diseases, such as inflammation and cancer.

### FIELD AND BACKGROUND OF THE INVENTION

The present invention, in some embodiments thereof, relates to the expression of necroptosis pathway components in exosomes and to their effect on endosomal trafficking. More particularly, but not exclusively, it relates to the use of same for diagnosis and therapeutic applications.

The recent four decades have seen immense progress in clarifying the molecular mechanisms for cell death. Initially, the study of the mechanisms of cell death focused on apoptosis, and on proteins such as the caspases and members of the Bcl2 family that contribute to this form of programmed death. More recently, knowledge has also been gained of proteins that contribute to induction of various forms of programmed necrotic death. Most detailed information has been gained of necroptosis, a form of programmed necrotic cell death mainly induced by extracellular inducers like tumor necrosis factor (TNF) [Wallach D. et al., Science (2016) 352: aaf2154]. The mediation of necroptotic death depends on the pseudokinase mixed lineage kinase domain-like protein (MLKL) and on its phosphorylation by the kinase RIPK3 [Sun L. et al., Cell (2012) 148: 213-227; Zhao J. et al., Proceedings of the National Academy of Sciences of the United States of America (2012) 109: 5322-5327]. The activation of RIPK3 itself is mediated, by some of the necroptosis inducers, including TNF, through stimulation of the kinase RIPK1 [Wallach et al. (2016), supra]. MLKL phosphorylation by RIPK3 exposes the N-terminal coiled-coil region of the former and thus imposes its oligomerization. Several studies suggested that death is mediated by association of the oligomerized MLKL molecules with distinct lipids in the plasma membrane and consequent formation of pores in the membrane, while others suggested other mechanisms for death mediation by MLKL [Czabotar P E and Murphy J M. FEBS J (2015) 282: 4268-4278].

Though initially conceived as processes whose molecular components are exclusively destined to induction of death, it is now known that programmed processes of death are actually mediated by proteins that serve other functions as well. The ‘death receptors’ of the TNF family, for example, also control numerous non-deadly functions. Some of the caspases that mediate death also promote cell growth and differentiation, etc. The occurrence of death in a programmed manner is therefore dictated, not only by the identity of the proteins that participate in its mediation, but also by the choice of the particular activities that these proteins exert, among several that they possess—a choice dictated by effects of contextual cues [Wallach et al. (2016), supra]. Identification of non-deadly functions of the proteins that mediate death, and elucidation of their interrelationship with the deadly functions of these proteins, are crucial for our understanding of the way death is controlled as well as for our ability to apply our molecular knowledge of death to monitor its occurrence in vivo.

U.S. Patent Application No. 2016/0160189 provides methods and compositions for inducing necroptosis in target cells, including cancer cells. Specifically, according to U.S. 2016/0160189 necroptosis is induced using compositions including oligomers comprising RIPK3 proteins and RIPK1 proteins including, but not limited to, full length RIPK3 homodimers, truncated RIPK3 oligomers and/or full length RIPK3/RIPK1 heterodimers.

### SUMMARY OF THE INVENTION

According to an aspect of some embodiments of the present invention there is provided a method of detecting activation of a necroptosis activation pathway in a subject, the method comprising: (a) obtaining a biological sample comprising exosomes from the subject; (b) detecting an activity or expression of a component of the necroptosis activation pathway in an exosome fraction of the biological sample, wherein when an increase in the activity or expression of the component of the necroptosis activation pathway in the exosome fraction is beyond a predetermined threshold with respect to an activity or expression of the component of the necroptosis activation pathway in an exosome fraction from a non-necroptotic sample is indicated the sample is considered as having the activation of the necroptosis activation pathway.

According to an aspect of some embodiments of the present invention there is provided a method of diagnosing a disease associated with activation of a necroptosis activation pathway in a subject, the method comprising: (a) detecting activation of a necroptosis activation pathway in a biological sample of the subject according to some embodiments of the invention; and (b) diagnosing the subject as having the disease associated with the activation of the necroptosis activation pathway when an increase in the activity or expression of the component of the necroptosis activation pathway in the exosome fraction is beyond a predetermined threshold with respect to an activity or expression of the component of the necroptosis activation pathway in an exosome fraction from a non-necroptotic sample.

According to an aspect of some embodiments of the present invention there is provided a method of detecting necroptosis or inflammation in a subject, the method comprising: (a) obtaining a biological sample comprising exosomes from the subject; (b) detecting a level of exosomes in the biological sample, wherein when an increase in the level is beyond a predetermined threshold with respect to a level of the exosomes in a biological sample from a non-necroptotic sample is indicated the sample is considered as a necroptotic or inflammatory sample.

According to an aspect of some embodiments of the present invention there is provided a method of diagnosing necroptosis or inflammation in a subject, the method comprising: (a) detecting a level of exosomes in a biological sample of the subject according to some embodiments of the invention; and (b) diagnosing the subject as having necroptosis or inflammation when an increase in the level of exosomes in the biological sample is beyond a predetermined threshold with respect to a level of the exosomes in a biological sample from a non-necroptotic sample.

According to an aspect of some embodiments of the present invention there is provided a method of identifying a tissue undergoing necroptosis in a subject, the method comprising: (a) obtaining a biological sample from the subject; (b) detecting an activity or expression of a component of a necroptosis activation pathway and an expression of a cell specific marker in an exosome fraction of the biological sample; (c) identifying the tissue undergoing necroptosis based on the measured level of the activity or expression of the component of the necroptosis activation pathway and the expression of the cell specific marker.

According to an aspect of some embodiments of the present invention there is provided a method of treating necroptosis in a subject in need thereof, the method comprising selecting a subject identified as having a necroptosis in accordance with the method of some embodiments of the invention, and administering an anti-necroptosis therapy to the subject.

According to an aspect of some embodiments of the present invention there is provided an anti-necroptosis therapy for use in treating necroptosis in a subject identified as having a necroptosis in accordance with the method of some embodiments of the invention.

According to an aspect of some embodiments of the present invention there is provided a method of treating an inflammation in a subject in need thereof, the method comprising selecting a subject identified as having an inflammation in accordance with the method of some embodiments of the invention, and administering an anti-inflammatory therapy to the subject.

According to an aspect of some embodiments of the present invention there is provided an anti-inflammatory therapy for use in treating an inflammation in a subject identified as having an inflammation in accordance with the method of some embodiments of the invention.

According to an aspect of some embodiments of the present invention there is provided a method of modulating endocytosis of a cell surface receptor capable of ligand induced endocytosis, the method comprising contacting a cell which expresses the cell surface receptor with an agent capable of downregulating an activity or expression of a MLKL, thereby modulating endocytosis of the cell surface receptor.

According to an aspect of some embodiments of the present invention there is provided a pharmaceutical composition comprising as an active ingredient an agent capable of downregulating an endocytic activity of MLKL without compromising necroptotic activity of the MLKL, and a pharmaceutically accepted carrier.

According to an aspect of some embodiments of the present invention there is provided an article of manufacture comprising an agent capable of downregulating an endocytic activity of MLKL without compromising necroptotic activity of the MLKL, and a ligand capable of binding to a cell surface receptor capable of ligand induced endocytosis, being packaged in a packaging material and identified in print, in or on the packaging material for use in the treatment of a disease or disorder in which modulating endocytosis of a cell surface receptor capable of ligand induced endocytosis is beneficial.

According to an aspect of some embodiments of the present invention there is provided a method of treating a disease or disorder in which modulating endocytosis of a cell surface receptor capable of ligand induced endocytosis is beneficial, the method comprising administering to a subject an agent capable of downregulating an endocytic activity of MLKL without compromising necroptotic activity of the MLKL, thereby treating the disease or disorder in the subject.

According to an aspect of some embodiments of the present invention there is provided an agent capable of downregulating an endocytic activity of MLKL without compromising necroptotic activity of the MLKL for use in treating a disease or disorder in which modulating endocytosis of a cell surface receptor capable of ligand induced endocytosis is beneficial.

According to an aspect of some embodiments of the present invention there is provided a method of enhancing immunotherapy in a subject in need thereof, the method comprising modulating endocytosis of a cell surface receptor capable of ligand induced endocytosis according to the method of some embodiments of the invention, wherein the ligand is capable of modulating T cell activation and enhancing an immune response.

According to an aspect of some embodiments of the present invention there is provided a modulator of endocytosis of a cell surface receptor for use in enhancing immunotherapy in a subject in need thereof.

According to an aspect of some embodiments of the present invention there is provided a pharmaceutical composition comprising as an active ingredient a population of exosomes comprising a component of a necroptosis activation pathway and a pharmaceutically accepted carrier.

According to an aspect of some embodiments of the present invention there is provided a method of inducing necroptosis or inflammation in a subject in need thereof, the method comprising administering to the subject a therapeutically effective amount of a population of exosomes comprising a component of a necroptosis activation pathway.

According to an aspect of some embodiments of the present invention there is provided a therapeutically effective amount of a population of exosomes comprising a component of a necroptosis activation pathway for use in inducing necroptosis or inflammation in a subject in need thereof.

According to some embodiments of the invention, the disease associated with the activation of the necroptosis activation pathway is selected from the group consisting of a necroptosis, an inflammation, a tissue damage, a tissue injury, a myocardinal infarction, a stroke, an ischemia-reperfusion injury (IRI), an atherosclerosis, a psoriasis, a pancreatitis, an inflammatory bowel disease, and a neurodegeneration.

According to some embodiments of the invention, the method further comprises administering to the subject an effective amount of an anti-necroptosis therapy or an anti-inflammatory therapy.

According to some embodiments of the invention, the method further comprises measuring an activity or expression of a component of a necroptosis activation pathway in the exosomes, wherein a ratio of the activity or expression of the component of the necroptosis activation pathway per level of exsosomes beyond a predetermined threshold is indicative of necroptosis or inflammation.

According to some embodiments of the invention, the detecting the activity or expression of the component of the necroptosis activation pathway in the exosome fraction of the biological sample is effected by contacting the biological sample with an agent targeting the component of the necroptosis activation pathway and detecting binding between the component of the necroptosis activation pathway and the agent.

According to some embodiments of the invention, the agent targeting the component of the necroptosis activation pathway is an antibody.

According to some embodiments of the invention, the detecting the expression of the cell specific marker in the exosome fraction of the biological sample is effected by contacting the biological sample with an agent targeting the cell specific marker and detecting binding between the cell specific marker and the agent.

According to some embodiments of the invention, the agent targeting the cell specific marker is an antibody.

According to some embodiments of the invention, the exosomes co-express the component of the necroptosis activation pathway and the cell specific marker.

According to some embodiments of the invention, the method further comprises purifying an exosome fraction of the biological sample prior to step (b).

According to some embodiments of the invention, the exosome fraction is essentially free of cells.

According to some embodiments of the invention, the biological sample is selected from the group consisting of a whole blood, a serum, a plasma, a saliva, a lymph, a urine, a semen and a milk sample.

According to some embodiments of the invention, the method further comprises analyzing the exosomes for expression of a cell specific marker.

According to some embodiments of the invention, the cell specific marker is selected from the group consisting of a protein, a RNA or of DNA.

According to some embodiments of the invention, the cell is selected from the group consisting of a cardiac, a spleen, a breast, a lung, a head, a neck, a prostate, an esophageal, a tracheal, a brain, a liver, a bladder, a stomach, a pancreatic, an ovarian, a uterine, a cervical, a testicular, a colon, a rectal, a kidney and a skin cell.

According to some embodiments of the invention, the necroptosis is associated with a disease selected from the group consisting of a tissue damage, a tissue injury, an inflammation, a myocardinal infarction, a stroke, an ischemia-reperfusion injury (IRI), an atherosclerosis, a psoriasis, a pancreatitis, an inflammatory bowel disease, and a neurodegeneration.

According to some embodiments of the invention, the tissue injury comprises an injury in an organ selected from the group consisting of a brain, a heart, a lung, a kidney, a liver, an intestine and a pancreas.

According to some embodiments of the invention, the anti-necroptosis therapy comprises an anti-inflammatory agent, an immunosuppressant agent, non-steroid anti-inflammatory drugs (NSAIDs) or a small molecule inhibitor of necroptosis.

According to some embodiments of the invention, the anti-necroptosis therapy comprises an agent for downregulating an activity or expression of at least one of MLKL, RIPK1, RIPK3, TNF-α or a Toll-like receptor ligand.

According to some embodiments of the invention, the agent for downregulating the activity or expression of the MLKL specifically compromises necroptotic activity of the MLKL without compromising an endocytic activity of the MLKL.

According to some embodiments of the invention, the inflammation is associated with a chronic inflammatory disease.

According to some embodiments of the invention, the inflammation is associated with an acute inflammatory disease.

According to some embodiments of the invention, the inflammation is associated with a disease selected from the group consisting of an infectious disease, an autoimmune disease, a hypersensitivity associated inflammation, a graft rejection and an injury.

According to some embodiments of the invention, the agent is capable of downregulating an endocytic activity of the MLKL without compromising a necroptotic activity of the MLKL.

According to some embodiments of the invention, the modulating the endocytosis of the cell surface receptor reduces intracellular degradation of the ligand.

According to some embodiments of the invention, the method further comprises contacting the cell with the ligand.

According to some embodiments of the invention, the pharmaceutical composition further comprises a ligand capable of binding to a cell surface receptor capable of ligand induced endocytosis.

According to some embodiments of the invention, the method further comprises administering to the subject the ligand.

According to some embodiments of the invention, the ligand is selected from the group consisting of a tumor necrosis factor (TNF) family member, an epidermal growth factor (EGF), an insulin, a thrombopoietin, a IL-18, a IL-23, a transforming growth factor beta (TGF-β), a neurotransmitter and a nucleic acid.

According to some embodiments of the invention, the ligand comprises a TNF family member.

According to some embodiments of the invention, the modulator of endocytosis is an agent capable of downregulating an activity or expression of a MLKL.

According to some embodiments of the invention, the agent is capable of downregulating an endocytic activity of the MLKL without compromising a necroptotic activity of the MLKL.

According to some embodiments of the invention, modulating the endocytosis of the cell surface receptor reduces intracellular degradation of a ligand.

According to some embodiments of the invention, the agent for use or modulator of endocytosis for use according to some embodiments of the invention further comprises the use of the ligand.

According to some embodiments of the invention, the disease or disorder is selected from the group consisting of a tumor, an immunodeficiency, an autoimmune disease, a diabetes, an inflammatory disease, a chronic infection, a neurodegenerative disease, a thrombocytopenia and a Chronic Obstructive Pulmonary Disease (COPD).

According to some embodiments of the invention, the component of the necroptosis activation pathway comprises a mixed lineage kinase domain-like protein (MLKL).

According to some embodiments of the invention, the MLKL comprises a phosphorylated MLKL.

According to some embodiments of the invention, the MLKL comprises a constitutively active mutant.

According to some embodiments of the invention, the phosphorylated MLKL comprises a phospho-mimetic mutation at an amino acid residue that is the target of phosphorylation by RIPK3.

According to some embodiments of the invention, the phospho-mimetic mutation comprises a threonine to glutamic acid modification in amino acid 357 and/or a serine to aspartic acid modification in amino acid 358 of the MLKL.

According to some embodiments of the invention, the phosphorylated MLKL comprises a phospho-mimetic mutation at an amino acid residue within the ATP-binding pocket of the MLKL.

According to some embodiments of the invention, the phospho-mimetic mutation comprises a lysine to methionine modification in amino acid 230 and/or a glutamine to alanine modification in amino acid 356 of the MLKL.

According to some embodiments of the invention, the component of the necroptosis activation pathway comprises a receptor interacting protein kinase 1 (RIPK1) or a receptor interacting protein kinase 3 (RIPK3).

According to some embodiments of the invention, the RIPK1 or RIPK3 comprises a phosphorylated RIPK1 or RIPK3.

According to some embodiments of the invention, the RIPK1 or RIPK3 comprises a constitutively active mutant.

According to some embodiments of the invention, the exosomes have a particle size of about 20 to about 200 nm.

According to some embodiments of the invention, the exosomes are genetically engineered.

According to some embodiments of the invention, the exosomes further comprise a binding agent on their surface for targeting to a diseased cell.

According to some embodiments of the invention, the binding agent is selected from the group consisting of a protein, a peptide and a glycolipid molecule.

According to some embodiments of the invention, the diseased cell is an inflammatory associated cell, a cancerous cell or a cell of a hyperproliferative disorder.

According to some embodiments of the invention, the subject has an inflammatory disease, a cancer, or a hyperproliferative disorder.

According to some embodiments of the invention, the subject is a human subject.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-I illustrate that triggering the necroptotic signaling pathway enhances generation of exosomes containing phospho-MLKL. FIG. 1A is a graph illustrating the extent of necroptotic cells death at different time points after TBZ application to HT29 cells. FIG. 1B is a photograph illustrating scanning electron microscopy of the particulate material released by HT-29 cells. FIG. 1C is a graph illustrating Nanoparticle Tracking Analysis size distribution of the vesicles generated by untreated and TBZ-treated HT-29 cells. FIG. 1D is a photograph illustrating western blot analysis of the impact of TBZ on the composition of the exosomes generated by HT29 cells—kinetic analysis. FIG. 1E is a photograph illustrating western blot analysis of the impact of TBZ and its individual components (applied for 4 hours) on the composition of the exosomes generated by HT29 cells. FIG. 1F is a photograph illustrating enhancement of exosome generation in HT-29 cells by TBZ and its reversal by necrostatin-1 (Nec-1). FIGS. 1G-I illustrate the in vivo effect of injection of mice with TNF+zVAD. FIG. 1G—Induction of necroptosis, as reflected in increased serum levels of lactic dehydrogenase (LDH). FIG. 1H—Increased plasma content of exosomes. FIG. 1I—Presence of MLKL and of phospho-MLKL in the mice plasma exosomes.

FIGS. 2A-H illustrate that exosome generation is impeded in MLKL deficient cells. FIGS. 2A-F: Effects of MLKL RNA silencing (in HepG2 cells and in MEFs derived from Ripk3^{-/-} mice) and of MLKL deficiency achieved by gene targeting (in BMDCs and in MEFs), and of ionomycin treatment (IONO; 1 mM for 12 hours) on exosome generation, as assessed by (FIGS. 2A-C and 2E) nanoparticle tracking analysis (NTA), and (FIG. 2D) by immunoblotting. FIG. 2F is a photograph illustrating a western blot analysis showing arrest of constitutive exosome generation by MLKL knockout, but not by knockout of RIPK¹ in mouse fibroblasts. FIG. 2G is a graph illustrating normal extent of constitutive generation of exosomes by three lines of RIPK3 deficient mouse fibroblasts. FIG. 2H is a photograph illustrating western blot analysis of RIPK3 levels in HT-29, HeLa and HepG2 cells.

FIGS. 3A-G illustrate the mutational exploration of the structural requirement for the roles of MLKL in necroptosis and in controlling endosomal trafficking. FIGS. 3A-E illustrate the yield of exosomes (empty bars) and extent of cell death (black bars) in MLKL knocked-down HT-29 cells inducibly expressing MLKL and its indicated mutants for FIG. 3A—3 hours and for FIGS. 3B, 3D, 3E—12 hours. FIGS. 3C and 3E are photographs illustrating western blot analyses of the expressed proteins. FIGS. 3F and 3G illustrate the effects of inducible expression of wild-type MLKL and its indicated mutants on the levels of EGF and EGFR in MLKL knocked-down HepG2 cells after binding of EGF to them. EGF was applied to the cells 7 hours after induction of the mutants was started. FIG. 3F is a photograph illustrating western blot analysis. FIG. 3G is a graph illustrating densitometric quantification of the results. Expression of the K230M/Q356A mutant and of the T357E/S358D mutant caused the death of only about 1% and no death, respectively, of HepG2 cells within the time of the test.

FIGS. 4A-D illustrate that MLKL controls the accumulation of intraluminal vesicles in multivesicular bodies. FIGS. 4A-C illustrate the transmission electron microscopy (TEM) of the MVBs in wild-type and MLKL knockdown HepG2 cells. (FIG. 4A) Representative pictures (bar, 100 nm), and FIGS. 4B and 4C illustrate the quantification of the sizes of arbitrarily chosen MVBs (132 MVBs in control cells and 115 in MLKL knocked-down cells), identified by the presence of BSA—gold in them (arrows), and of their ILV content. FIG. 4D is a photograph illustrating an immunoelectron microscopic analysis of EGFR uptake into MVB in control and MLKL knocked-down HepG2 cells. Black arrows, BSA tagged with 5 nm gold particles, whose uptake by the cells served to mark the endosomal system; red arrows, EGFR, detected by antibodies tagged with 12 nm gold particles; black arrowheads, CD63, a marker of late endosomes and of MVB, detected by antibodies tagged with 18 nm gold particles.

FIGS. 5A-G illustrate that MLKL controls transport to the late endosomes. FIG. 5A is a photograph of western blot analysis of the kinetics of intracellular degradation of biotinylated TNF following its binding to the indicated cells, and the impact of MLKL knockdown on it. FIG. 5B is a photograph of western blot analysis of the kinetics of intracellular degradation of TNF in HT-29 cells, and the impact of MLKL knockdown on it. Also shown are the cellular levels of several targets of TNF receptor signaling—phosphorylated p38 as well as ERK, IκBα, p65 and their phosphorylated forms. FIG. 5C shows the effect of MLKL RNA silencing on the TNF-induced expression of inflammation-related genes, as assessed with the NanoString nCounter Analysis System. Control or MLKL-siRNA-silenced HT29 cells were treated with TNF for the indicated times. Numbers in the table record the fold increase in expression of the specified genes relative to control cells at 0 hours. Shown are the 20 most strongly upregulated genes (averages of duplicate tests), sorted in descending order for the 6 hour siRNA-silenced sample. Darker fill color indicates higher expression. FIG. 5D presents Real-time PCR validation of the expression kinetics of genes analyzed in (C) by the NanoString system. FIG. 5E is a photograph of western blot analysis of the kinetics of intracellular degradation of EGF and of the EGFR in HepG2 cells, and the impact of MLKL knockdown on them. Also shown are the cellular levels of phosphorylated EGFR and of three targets of EGFR signaling—AKT, STAT3, ERK—and their phosphorylated forms. FIG. 5F is a photograph showing the impact of MLKL deficiency on EGF and EGFR degradation and on EGFR signaling in the livers of wild-type and MLKL-knockout mice at various times after injection of biotinylated EGF. FIG. 5G presents real-time PCR analysis of the expression kinetics of several EGF-induced genes in the livers of mice, 2 hours after their injection with EGF.

FIGS. 6A-C present evidence that endosomal trafficking is slowed in MLKL-depleted cells. FIG. 6A presents immunofluorescence images. Left and right panels (of each set): immunocytochemical analysis of EGFR uptake after application of EGF to control and MLKL-siRNA-silenced HepG2 cells, and the kinetics of colocalization of the receptor with EEA1 and Rab7-markers of early and late endosomes, respectively. Yellow, co-localization of EGFR (green) and EEA1 (red); cyan, co-localization of EGFR (green) and Rab7 (blue). Scale bar, 10 mm. FIGS. 6B and 6C present quantification of the data presented in FIG. 6A. FIG. 6B—Total amounts of EGFR in the cells and the amounts of EGFR associated with the cell membrane at different times, expressed as percentages of the initial total amounts of EGFR in the cells. Values are averages of two independent experiments. The bars show the range of the results. FIG. 6C amounts of EEA1 and Rab7 that colocalize with EGFR expressed respectively as percentages of the total amounts of EEA1 and Rab7 in the cells. Quantification of the data presented in FIG. 6A.

FIG. 7 is a photograph illustrating an immunocytochemical analysis of a test performed as in FIG. 6, using lysotracker (a lysosome-staining reagent) and antibodies to EGFR and to the late endosomal marker Rab7. Shown are the results of immunostaining and their superposition on transmission pictures. Cyan arrows indicate co-localization of EGFR and Rab7 staining; white arrows indicate co-localization of EGFR, lysotracker, and Rab7 staining. At the time of the test (2 hours after ligand binding) the EGFRs taken up were almost fully degraded in the control cells, whereas in the MLKL knocked-down cells some EGFR remained in the late endosome compartment. Arrest of lysosomal degradation by CQ or Baf A1 allowed equal accumulation of EGFR in that compartment in the control and MLKL knocked-down cells. Bar, 10 μM.

FIGS. 8A-E illustrate that exosomes released from cells in which the necroptotic pathway is activated can serve as mediators of inflammation. FIG. 8A is a graph illustrating the yield of IL-1β in response to activation of the necroptotic pathway in dendritic cells as compared to the extent of death induction. FIG. 8B is a graph illustrating that activation of the necroptotic pathway by LPS in bone marrow derived dendritic cells enhanced exosome release. FIGS. 8C-D are photographs of western blot analysis revealing that these exosomes contained phospho-MLKL as well IL-1β and the processed form of caspase-1, the enzyme activating IL-1β. FIG. 8E is a graph showing that when applied to bone marrow derived macrophages, these exosomes triggered expression of the gene encoding the inflammatory cytokine IL-6. Of note, WT—wild-type, and C8 KO—caspase-8 deficient cells.

FIG. 9 is a schematic illustration of the effect of MLKL on endosomal trafficking and its roles in mediation of necroptosis and inflammation.

### DESCRIPTION OF SPECIFIC EMBODIMENTS OF THE INVENTION

The present invention, in some embodiments thereof, relates to the expression of necroptosis pathway components in exosomes and to their effect on endosomal trafficking. More particularly, but not exclusively, it relates to the use of same for diagnosis and therapeutic applications.

The principles and operation of the present invention may be better understood with reference to the drawings and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

The pseudo protein kinase Mixed Lineage Kinase Domain-Like (MLKL) mediates necroptosis—a form of programmed necrotic cell death. This activity depends on its phosphorylation by RIPK3—a protein kinase activated by signaling pathways triggered by death-inducing agents such as TNF or Toll-like receptor ligands.

While reducing the present invention to practice, the present inventors have surprisingly uncovered that the necroptosis activation pathway is associated with cellular mechanisms not directly related to cell death.

The present inventors have uncovered that the necroptotic signaling pathway enhances generation of exosomes and that these exosomes comprise activated components of the MLKL pathway. As such exosomes and components of the necroptosis activation pathway can be used as a measure of necroptosis in general and as a clinical tool for diagnosis and treatment of necroptosis associated diseases.

Specifically, the present inventors have surprisingly uncovered that triggering the necroptotic signaling pathway enhances generation of exosomes (see Example 1 of the Examples section which follows) while knockdown of components of the necroptosis signaling pathway, e.g. MLKL, RIPK1 or RIPK3, significantly reduced generation of exosomes (see Examples 1 and 2).

Whilst further reducing embodiments of the invention to practice, the present inventors uncovered a central role for MLKL in ligand-induced receptor endocytosis. This activity was found independent of its necroptotic activity. These results suggest that regulating MLKL can be used as a measure for controlling signaling of ligand induced cell surface molecules, suggesting role in disease control.

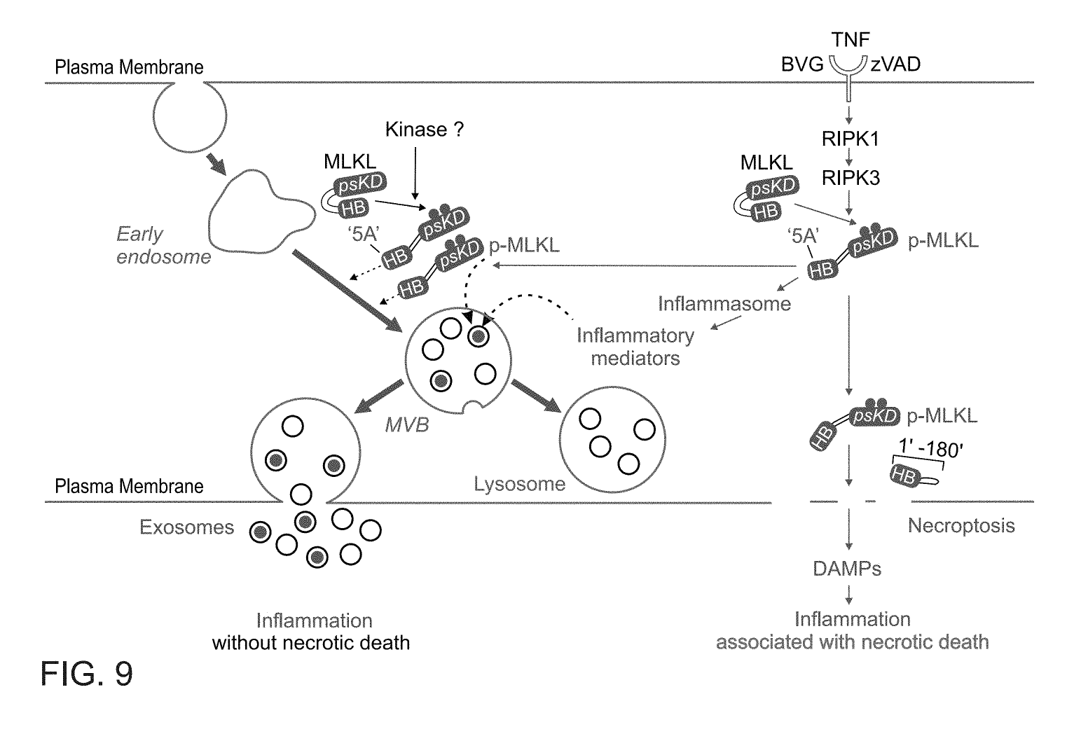

Specifically, the present inventors uncovered that MLKL, independently of death induction or of the functions of RIPK1 and RIPK3, also serves to regulate endosomal trafficking, thereby facilitating the transport of endocytosed proteins to intraluminal vesicles (ILVs) within the multivesicular bodies (MVBs) (see Examples 4 and 5 and FIG. 9). Deficiency of MLKL results in marked reduction in ILV generation, slowdown of lysosomal degradation of endocytosed proteins, and in a marked potentiation of the effects of extracellular ligands (e.g. TNF) (Example 7). Conversely, activation of the necroptotic pathway by RIPK3-mediated phosphorylation of MLKL results in enhanced lysosomal degradation, increased exosomal generation, and release of the phosphorylated MLKL as well as RIPK1 and RIPK3 within exosomes (see Example 1). Mutational analysis suggested that the structural requirements for the control of endosomal function by MLKL are in part identical with those for necroptosis and in part distinct from them (see Example 3).

Taken together, these results illustrate the diagnostic and therapeutic potential of components of the necroptosis activation pathway in exosomes and in endosomal trafficking.

Thus, according to one aspect of the present invention there is provided a method of detecting activation of a necroptosis activation pathway, the method comprising: detecting an activity or expression of a component of the necroptosis activation pathway in an exosome fraction of a biological sample comprising exosomes, wherein when an increase in the activity or expression of the component of the necroptosis activation pathway in the exosome fraction is beyond a predetermined threshold with respect to an activity or expression of the component of the necroptosis activation pathway in an exosome fraction from a non-necroptotic sample is indicated the sample is considered as having the activation of the necroptosis activation pathway.

Thus, according to an alternative or an additional aspect of the present invention there is provided a method of detecting activation of a necroptosis activation pathway in a subject, the method comprising: (a) obtaining a biological sample comprising exosomes from the subject; and (b) detecting an activity or expression of a component of the necroptosis activation pathway in an exosome fraction of the biological sample, wherein when an increase in the activity or expression of the component of the necroptosis activation pathway in the exosome fraction is beyond a predetermined threshold with respect to an activity or expression of the component of the necroptosis activation pathway in an exosome fraction from a non-necroptotic sample is indicated the sample is considered as having the activation of the necroptosis activation pathway.

According to an alternative or an additional aspect of the present invention there is provided a method of detecting necroptosis or inflammation in a subject, the method comprising: (a) obtaining a biological sample comprising exosomes from the subject; and (b) detecting a level of exosomes in the biological sample, wherein when an increase in the level is beyond a predetermined threshold with respect to a level of the exosomes in a biological sample from a non-necroptotic sample is indicated the sample is considered as a necroptotic or inflammatory sample.

The term “necroptosis” as used herein refers to a programmed necrotic cell death. Necroptosis is also referred to as necrosis associated with inflammation. Characteristically, necroptosis involves cellular swelling and rupture, thereby releasing the intracellular contents.

Necroptosis is typically evident by signs of cell death and tissue damage. These can be detected by imaging (e.g. MRI, CT, ultrasound etc.). Furthermore, cytoplasmic components, such as the enzyme lactic dehydrogenase, or inflammatory mediators, such as IL-1β, can be found at the site of necroptosis. These can be detected by typical blood tests or in biopsy samples. Necroptosis is also typically evident by activation of the necroptotic pathway, which serves as a marker for necroptosis. An increased expression or activity level of at least one component of the necroptosis activation pathway (e.g. RIPK1, RIPK3, MLKL, discussed in detail below) can be analyzed by any method known in the art, e.g. by western blot analysis. Alternatively or additionally, necroptosis can be detected by staining for dead cells by e.g. Annexin-V and 7-amino actinomycin D (or propidium iodine) and analyzing the stained cells (e.g. by FACS). Typically, cells that stain positive for both Annexin-V and 7AAD are considered not intact (e.g. necroptotic) Likewise, necroptosis can be identified by dual staining of cells for dichloro-dihydro-fluorescein diacetate (DCFH-DA) and propidium iodide and analyzing the stained cells (e.g. by FACS). Typically, cells that stain positive for both DCFH-DA and propidium iodide are considered necroptotic. Cell death can also be quantified using the Cytotoxicity Detection Kit (such as the one available from Roche Applied Science). Any of these can be used to corroborate necroptosis according to some embodiments of the invention.

Without being bound to theory, necroptosis typically begins by binding of extracellular inducers like tumor necrosis factor (TNF), ligands of Toll-like receptor (TLR) or interferon to their cellular receptors (e.g. TNF receptor, TLR). In the case of some of these inducers, this binding triggers stimulation of receptor interacting kinase 1 (RIPK1), which in turn activates receptor interacting protein kinase 3 (RIPK3), which phosphorylates and activates the pseudokinase mixed lineage kinase domain-like protein (MLKL). In turn, MLKL mediates cell death (e.g. via loss of cell membrane integrity) mediating the release of cellular contents (e.g. products of cell death). Other inducers activate RIPK3 in other ways. For example, initiation of the necroptosis activation pathway can begin by binding of viral factors to DNA-dependent activator of interferon regulatory factors (DAI). DAI interacts with RIPK3 to mediate virus-induced necrosis analogous to the RIPK1-RIPK3 pathway.

Inflammation as used herein, is an aspect of many diseases and disorders, also referred to inflammatory diseases, including but not limited to diseases related to immune disorders, viral and bacterial infection, arthritis, autoimmune diseases, collagen diseases, allergy, asthma, pollinosis, cancer and atopy (as described in further detail below).

The phrase “necroptosis activation pathway” as used herein refers to the signaling pathway which leads to necroptosis of a cell.

The phrase “component of the necroptosis activation pathway” as used herein refers to any cellular component (e.g., mRNA, protein or metabolite) involved in signaling in the necroptosis pathway including, but not limited to, RIPK1, RIPK3 and MLKL. An illustration of the necroptosis pathway is described e.g. in Belizário et al., Mediators of Inflammation (2015) pages 1-15.

The term “RIPK1” refers to the human Receptor Interacting Serine/Threonine Kinase 1 also termed Receptor-Interacting Protein Kinase 1 or Receptor-Interacting Protein 1, a product of Gene ID: 8737. Exemplary RIPK1 polypeptides are set forth in GenBank accession nos. NP_001303990.1 and NP_003795.2.

The term “RIPK3” refers to the human Receptor Interacting Serine/Threonine Kinase 3 also termed Receptor-Interacting Protein Kinase 3 or Receptor-Interacting Protein 3, a product of Gene ID: 11035. Exemplary RIPK3 polypeptides are set forth in GenBank accession no. NP_006862.2 and EC 2.7.11.1.

The term “MLKL” refers to the Mixed Lineage Kinase Domain-Like protein, a product of Gene ID: 197259. Exemplary MLKL polypeptides are set forth in GenBank accession nos. NP_001135969.1 and NP_689862.1.

The phrase “activation of a necroptosis activation pathway” refers to the state wherein a component of the necroptosis activation pathway, e.g. RIPK1, RIPK3 and/or MLKL, is transformed from a latent form to an active form.

According to another embodiment, activation of a necroptosis activation pathway is embodied by enhanced expression of a component of the necroptosis activation pathway, e.g. RIPK1, RIPK3 and/or MLKL (e.g. in an exosome).

According to one embodiment, activation of a necroptosis activation pathway is embodied by phosphorylation of a component of the necroptosis activation pathway e.g. RIPK1, RIPK3 and/or MLKL.

According to another embodiment, activation of a necroptosis activation pathway is embodied by an increase in the normalized phophorylation of a component of the necroptosis activation pathway, e.g. RIPK1, RIPK3 and/or MLKL, i.e. an increase in the ratio of phosphorylation per level of exosomes.

Detecting activation of a necroptosis pathway is typically carried out by first obtaining a biological sample comprising exosomes (e.g., from a subject in need thereof, as further described hereinbelow) and analyzing the biological sample for an activity or expression of a component of the necroptosis activation pathway in an exosome fraction of the biological sample.

As used herein, the terms “subject” or “subject in need thereof” include mammals, preferably human beings at any age or gender. The subject may be healthy or showing preliminary signs of a pathology, e.g. a pathology associated with a necroptosis activation pathway, e.g., with inflammation. This term also encompasses individuals who are at risk to develop the pathology (e.g. due to a tissue damage, tissue injury, stroke, infectious disease or organ transplant).

As used herein “a biological sample” refers to a biological sample (e.g., fluid or hard tissue) which comprises exosomes. Examples of fluid samples include, but are not limited to, whole blood, plasma, serum, spinal fluid, lymph fluid, bone marrow suspension, cerebrospinal fluid, brain fluid, ascites (e.g. malignant ascites), tears, saliva, sweat, urine, semen, sputum, ear flow, vaginal flow, secretions of the respiratory, intestinal and genitourinary tracts, milk, amniotic fluid, and samples of in vivo cell culture constituents. Examples of tissue samples include, but are not limited to, surgical samples, biopsy samples, tissues, feces, and cultured cells.

Methods of obtaining such biological samples are known in the art, and include without being limited to, standard blood retrieval procedures, standard urine and semen retrieval procedures, lumbar puncture, fine needle biopsy, needle biopsy, core needle biopsy and surgical biopsy (e.g., organ or brain biopsy), buccal smear and lavage. Regardless of the procedure employed, once a biopsy/sample is obtained the level of the variant can be determined and a diagnosis can thus be made.

According to one embodiment, the biological sample comprises exosomes (or is further processed to comprise exosomes, as discussed below) and is essentially without intact cells.

According to a specific embodiment, the biological sample (e.g. processed sample) comprises less than 1%, 2%, 5%, 10%, 20%, 30%, 40%, 50%, 60%, 70%, 80% or 90% intact cells per ml fluid sample.

However, the biological sample may contain some cells or cell contents. The cells can be any cells which are derived from the subject. Examples include, but are not limited to, blood cells, bone marrow cells, brain cells, hepatic cells, spleen cells, kidney cells, cardiac cells, skin cells (e.g., epithelial cells, fibroblasts, keratinocytes), lymph node cells, and fetal cells such as amniotic cells, placental cells (e.g., fetal trophoblasts) and/or cord blood cells.

The term “exosomes” as used herein refers to externally released vesicles originating from the endosomic compartment of cells. Exosomes typically have a particle size of about 20-200 nm (e.g. about 30-100 nm) and are released from many different cell types, including but not limited to, tumor cells, red blood cells, platelets, immune cells (e.g. antigen presenting cells, dendritic cells, macrophages, mast cells, T lymphocytes or B lymphocytes), kidney cells, hepatic cells, cardiac cells, lung cells, spleen cells, pancreatic cells, brain cells, skin cells, mesenchymal stem cells (e.g. human umbilical cord MSCs) and other cell types.

According to one embodiment, the exosomes originate from cells undergoing necroptosis or inflammation.

Typically, exosomes are formed by invagination and budding from the limiting membrane of late endosomes. They accumulate in cytosolic multivesicular bodies (MVBs) from where they are released by fusion with the plasma membrane. Alternatively, vesicles similar to exosomes (though somewhat larger, often called ‘microvesicles’) can be released directly from the plasma membrane. The process of vesicle shedding is particularly active in proliferating cells, such as cancer cells, where the release can occur continuously. Depending on the cellular origin, exosomes harbor biological material including e.g. nucleic acids (e.g. RNA or DNA), proteins, peptides, polypeptides, antigens, lipids, carbohydrates, and proteoglycans. For example, various cellular proteins can be found in exosomes including MHC molecules, tetraspanins, adhesion molecules and metalloproteinases.

The volume of the biological sample used for analyzing exosomes can be in the range of between 0.1-100 mL, such as less than about 100, 75, 50, 25, 15, 10, 9, 8, 7, 6, 5, 4, 3, 2, 1 or 0.1 mL.

The biological sample of some embodiments of the invention may comprise any number of exosomes, e.g. 1, 5, 10, 15, 20, 25, 50, 100, 150, 200, 250, 500, 1000, 2000, 5000, 10,000, 50,000, 100,000, 500,000, 1×10⁶ or more exosomes.

As used herein, the term “exosome fraction” relates to the fraction of the biological sample comprising the exosomes.

According to one embodiment, the exosome fraction comprises exosomes and is free of cells (as discussed above).

According to one embodiment, exosomes are obtained from a freshly collected biological sample or from a biological sample that has been stored frozen or refrigerated.

Exosomes can be isolated from the biological sample by any method known in the art. Suitable methods are taught, for example, in U.S. Pat. Nos. 9,347,087 and 8,278,059, incorporated herein by reference.

For example, exosomes may be obtained from a fluid sample by first removing any debris from the sample e.g. by precipitation with a volume-excluding polymer (e.g. polyethylene glycol (PEG) or dextrans and derivatives such as dextran sulfate, dextran acetate, and hydrophilic polymers such as polyvinyl alcohol, polyvinyl acetate and polyvinyl sulfate). Methods of clarification include centrifugation, ultracentrifugation, filtration or ultrafiltration. The skilled artisan is aware of the fact, that an efficient separation might require several centrifugation steps using different centrifugation procedures, temperatures, speeds, durations, rotors, and the like. For example, suitable volume-excluding polymers may have a molecular weight between 1000 and 1,000,000 daltons. In general, when higher concentrations of exosomes are present in a sample, lower molecular weight polymers may be used. Volume-excluding polymers may be used at a final concentration of from 1% to 90% (w/v) upon mixing with the sample. A variety of buffers commonly used for biological samples may be used for incubation of the exosome sample with the volume-excluding polymer including phosphate, acetate, citrate and TRIS buffers. The pH of the buffer may be any pH that is compatible with the sample, but a typical range is from 6 to 8. Incubation of the biological sample with the volume-excluding polymer may be performed at various temperatures, e.g. 4° C. to room temperature (e.g. 20° C.). The time of incubation of the sample with the volume-excluding polymer may be any, typically in the range 1 minute to 24 hours (e.g. 30 minutes to 12 hours, 30 minutes to 6 hours, 30 minutes to 4 hours, or 30 minutes to 2 hours). One of skill in the art is aware that the incubation time is influenced by, among other factors, the concentration of the volume-excluding polymer, the molecular weight of the volume-excluding polymer, the temperature of incubation and the concentration of exosomes and other components in the sample. After completion of the incubation of the sample with the volume-excluding polymer the precipitated exosomes may be isolated by centrifugation, ultracentrifugation, filtration or ultrafiltration.

According to one embodiment, exosomes are separated from a biological fluid sample by first centrifugation of the biological sample (e.g. fluid sample such as plasma) at 3000 rpm for 15 minutes, then passing the sample through a filter (e.g. 0.1-0.5 μm filter, e.g. 0.2 μm filter) and centrifugation at about 10,000×g for 60-120 minutes (e.g. 90 minutes). Centrifugation can be repeated (e.g. after suspending the pellet in phosphate-buffered saline (PBS)) under the same conditions.

When isolating exosomes from tissue sources it may be necessary to homogenize the tissue in order to obtain a single cell suspension followed by lysis of the cells to release the exosomes. When isolating exosomes from tissue samples it is important to select homoginazation and lysis procedures that do not result in disruption of the exosomes.

The exosomal fraction may be further purified or concentrated prior to analysis. For example, a heterogeneous population of exosomes can be quantitated (i.e. total level of exosomes in a sample), or a homogeneous population of exosomes, such as a population of exosomes with a particular size, with a particular marker profile, obtained from a particular type of biological sample (e.g. urine, serum, plasma, etc.) or derived from a particular cell type (e.g. expressing a cell specific marker as described in detail below) can be isolated from a heterogeneous population of exosomes and quantitated.

For example, exosomes may be purified or concentrated from a biological sample using size exclusion chromatography, density gradient centrifugation, differential centrifugation, nanomembrane ultrafiltration, immunoabsorbent capture, affinity purification, microfluidic separation, or combinations thereof.

Size exclusion chromatography, such as gel permeation columns, centrifugation or density gradient centrifugation, and filtration methods can be used. For example, exosomes can be isolated by differential centrifugation, anion exchange and/or gel permeation chromatography (as described e.g. in U.S. Pat. Nos. 6,899,863 and 6,812,023), sucrose density gradients, organelle electrophoresis (as described e.g. in U.S. Pat. No. 7,198,923), magnetic activated cell sorting (MACS), or with a nanomembrane ultrafiltration concentrator. Thus, various combinations of isolation or concentration methods can be used as known to one of skill in the art.

Sub-populations of exosomes may also be isolated by using other properties of the exosomes such as the presence of surface markers. Surface markers which may be used for fraction of exosomes include but are not limited to tumor markers, cell type specific markers and MHC class II markers. MHC class II markers which have been associated with exosomes include HLA DP, DQ and DR haplotypes. Other surface markers associated with exosomes include, but are not limited to, CD9, CD81, CD63, CD82, CD37, CD53, or Rab-5b (Thery et al. Nat. Rev. Immunol. 2 (2002) 569-579; Valadi et al. Nat. Cell. Biol. 9 (2007) 654-659).

As an example, exosomes having CD63 on their surface may be isolated using antibody coated magnetic particles e.g. using Dynabeads®, super-paramagnetic polystyrene beads which may be conjugated with anti-human CD63 antibody either directly to the bead surface or via a secondary linker (e.g. anti-mouse IgG). The beads may be between 1 and 4.5 μm in diameter. Accordingly, the antibody coated Dynabeads® may be added to an exosome sample (e.g. prepared as described above) and incubated at e.g. 2-8° C. or at room temperature from 5 minutes to overnight. Dynabeads® with bound exosomes may then be collected using a magnet. The isolated, bead bound exosomes may then be resuspended in an appropriate buffer such as phosphate buffered saline and used for analysis (qRT-PCR, sequencing, western blot, ELISA, flow cytometry, etc. as discussed below). Similar protocols may be used for any other surface marker for which an antibody or other specific ligand is available. Indirect binding methods such as those using biotin-avidin may also be used.

Determining the level of exosomes in a sample can be carried out using any method known in the art, e.g. by ELISA, using commercially available kits such as, for example, the ExoQuick kit (System Biosciences, Mountain View, Calif.), magnetic activated cell sorting (MACS) or by FACS using an antigen or antigens which bind general exosome markers, such as but not limited to, CD63, CD9, CD81, CD82, CD37, CD53, or Rab-5b.

According to one embodiment, once an isolated exosome sample (i.e. exosome fraction) has been prepared it can be stored, such as in a sample bank and retrieved for analysis as necessary, alternatively, the exosome fraction can be analyzed without storing the sample.

According to one embodiment, the exosomes are analyzed as a whole (i.e. without damaging the exosomal membrane).

According to another embodiment, the contents of the exosomes are extracted for study and characterization. Biological material which may be extracted from exosomes includes, for example, proteins, peptides, polypeptides, nucleic acids (e.g. RNA or DNA) and lipids. For example the mirVana™ PARIS Kit (AM1556, Life Technologies) or the ME™ Kit for Exosome Isolation may be used to recover native protein and RNA species, including small RNAs such as miRNA, snRNA, and snoRNA, from exosomes.

For example, total RNA may be extracted using acid-phenol:chloroform extraction. RNA may then be purified using a glass-fiber filter under conditions that recover small-RNA containing total RNA, or that separate small RNA species less than 200 nucleotides in length from longer RNA species such as mRNA. Because the RNA is eluted in a small volume, no alcohol precipitation step may be required for isolation of the RNA.

As taught by the present invention, components of the necroptosis activation pathway can be found in exosomes (e.g. RIPK1, RIPK3, MLKL, or phosphorylated forms thereof).

Detection of an activity or expression of a component of the necroptosis activation pathway in an exosome fraction can be carried out using any method known in the art, e.g. on the polypeptide level or on the transcript level.

Following is a non-limiting list of examples of methods of determining the activity or expression of a component of the necroptosis activation pathway on the polypeptide level.

Enzyme linked immunosorbent assay (ELISA): This method involves a reaction between an enzyme and a substrate. A biological sample which comprises a component of the necroptosis activation pathway (e.g. exosome fraction disrupted using detergent) is put in a microwell dish. A specific antibody (e.g. capable of targeting a component of the necroptosis activation pathway) coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western blot: This method involves separation of a substrate from other protein by means of an acrylamide gel followed by transfer of the substrate to a membrane (e.g., nylon or PVDF). Presence of the substrate is then detected by antibodies specific to the substrate (e.g. an antibody capable of targeting a component of the necroptosis activation pathway), which are in turn detected by antibody binding reagents. Antibody binding reagents may be, for example, protein A, or other antibodies. Antibody binding reagents may be radiolabeled or enzyme linked as described hereinabove. Detection may be by autoradiography, colorimetric reaction or chemiluminescence. This method allows both quantitation of an amount of substrate and determination of its identity by a relative position on the membrane which is indicative of a migration distance in the acrylamide gel during electrophoresis.

Radio-immunoassay (RIA): In one version, this method involves precipitation of the desired protein (i.e., the substrate) with a specific antibody capable of targeting a component of the necroptosis activation pathway, and radiolabeled antibody binding protein (e.g., protein A labeled with I¹²⁵) immobilized on a precipitable carrier such as agarose beads. The number of counts in the precipitated pellet is proportional to the amount of substrate.

In an alternate version of the RIA, a labeled substrate and an unlabelled antibody binding protein are employed. A sample containing an unknown amount of substrate is added in varying amounts. The decrease in precipitated counts from the labeled substrate is proportional to the amount of substrate in the added sample.

Fluorescence activated cell sorting (FACS): This method involves detection of a substrate in situ in exosomes by substrate specific antibodies i.e., antibodies capable of targeting a component of the necroptosis activation pathway. The substrate specific antibodies are linked to fluorophores. Detection is by means of a cell sorting machine which reads the wavelength of light emitted from each cell as it passes through a light beam. This method may employ two or more antibodies simultaneously.

Immunohistochemical analysis: This method involves detection of a substrate in situ in fixed exosomes by substrate specific antibodies, i.e., antibodies capable of targeting a component of the necroptosis activation pathway. The substrate specific antibodies may be enzyme linked or linked to fluorophores. Detection is by microscopy and subjective or automatic evaluation. If enzyme linked antibodies are employed, a colorimetric reaction may be required. It will be appreciated that immunohistochemistry is often followed by counterstaining of the cell nuclei using for example Hematoxyline or Giemsa stain.

In situ activity assay: According to this method, a chromogenic substrate is applied on the exosomes containing an active enzyme and the enzyme catalyzes a reaction in which the substrate is decomposed to produce a chromogenic product visible by a light or a fluorescent microscope.

In vitro activity assays: In these methods the activity of a particular enzyme is measured in a protein mixture extracted from the exosomes. The activity can be measured in a spectrophotometer well using colorimetric methods or can be measured in a non-denaturing acrylamide gel (i.e., activity gel). Following electrophoresis the gel is soaked in a solution containing a substrate and colorimetric reagents. The resulting stained band corresponds to the enzymatic activity of the protein of interest. If well calibrated and within the linear range of response, the amount of enzyme present in the sample is proportional to the amount of color produced. An enzyme standard is generally employed to improve quantitative accuracy.

According to one embodiment, for detection of RIPK1, anti-RIPK1 antibodies can be used (which detect the phosphorylated or non-phosphorylated form thereof) which can be commercially bought from e.g. OriGene (e.g. TA306838 and TA319759), Abcam (e.g. Q13546), Cloud-Clone Corp. (e.g. MAE640Hu21 and PAE640Hu01), GeneTex and Santa Cruz Biotechnology (e.g RIP (K-20) and RIP (H-207)).

According to one embodiment, for detection of RIPK3, anti-RIPK3 antibodies can be used (which detect the phosphorylated or non-phosphorylated form thereof) which can be commercially bought from e.g. OriGene (e.g. TA306042 and TA337010), Abcam (e.g. Q9Y572), Cloud-Clone Corp. (e.g. MAE639Hu21 and PAE639Hu01), GeneTex and Santa Cruz Biotechnology (e.g. RIP3 (N-14) and RIP3 (Rippy-3)).

According to one embodiment, for detection of MLKL, anti-MLKL antibodies can be used (which detect the phosphorylated or non-phosphorylated form thereof) which can be commercially bought from e.g. OriGene (e.g. TA316215), GeneTex, Santa Cruz Biotechnology (e.g. MLKL (Q-15) and MLKL (Y-14)), and EMD Millipore.

Following is a non-limiting list of examples of methods of determining the expression of a component of the necroptosis activation pathway on the transcript level.

The presence and/or level of a component of the necroptosis activation pathway nucleic acid sequence (e.g. RIPK1, RIPK3 or MLKL transcript) can be determined using an isolated polynucleotide (e.g., a polynucleotide probe, an oligonucleotide probe/primer) capable of hybridizing to a nucleic acid sequence of a component of the necroptosis activation pathway. Such a polynucleotide can be at any size, such as a short polynucleotide (e.g., of 15-200 bases), and intermediate polynucleotide (e.g., 200-2000 bases) or a long polynucleotide larger of 2000 bases.

The isolated polynucleotide probe used by the present invention can be any directly or indirectly labeled RNA molecule (e.g., RNA oligonucleotide, an in vitro transcribed RNA molecule), DNA molecule (e.g., oligonucleotide, cDNA molecule, genomic molecule) and/or an analogue thereof [e.g., peptide nucleic acid (PNA)] which is specific to the RNA transcript of the present invention.

Oligonucleotides designed according to the teachings of the present invention can be generated according to any oligonucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988) and “Oligonucleotide Synthesis” Gait, M. J., ed. (1984) utilizing solid phase chemistry, e.g. cyanoethyl phosphoramidite followed by deprotection, desalting and purification by for example, an automated trityl-on method or HPLC.

The above-described polynucleotides can be employed in a variety of transcript detection methods. Following is a non-limiting list of RNA-based hybridization methods which can be used to detect a component of the necroptosis pathway of the present invention.

Northern Blot analysis—This method involves the detection of a particular RNA in a mixture of RNAs. An RNA sample is denatured by treatment with an agent (e.g., formaldehyde) that prevents hydrogen bonding between base pairs, ensuring that all the RNA molecules have an unfolded, linear conformation. The individual RNA molecules are then separated according to size by gel electrophoresis and transferred to a nitrocellulose or a nylon-based membrane to which the denatured RNAs adhere. The membrane is then exposed to labeled DNA, RNA or oligonucleotide (composed of deoxyribo or ribonucleotides) probes. Probes may be labeled using radio-isotopes or enzyme linked nucleotides. Detection may be using autoradiography, colorimetric reaction or chemiluminescence. This method allows both quantitation of an amount of particular RNA molecules and determination of its identity by a relative position on the membrane which is indicative of a migration distance in the gel during electrophoresis.

Reverse-transcribed PCR (RT-PCR) analysis—This method is performed using specific primers. It will be appreciated that a semi-quantitative RT-PCR reaction can be also employed by adjusting the number of PCR cycles and comparing the amplification product to known controls. Alternatively, quantitative RT-PCR can be performed using, for example, the Light Cycler™ (Roche).

RNA in situ hybridization stain—In this method DNA, RNA or oligonucleotide (composed of deoxyribo or ribonucleotides) probes are attached to the RNA molecules present in the exosomes. Generally, the exosomes are first fixed to microscopic slides to preserve the cellular structure and to prevent the RNA molecules from being degraded and then are subjected to hybridization buffer containing the labeled probe. The hybridization buffer includes reagents such as formamide and salts (e.g., sodium chloride and sodium citrate) which enable specific hybridization of the DNA or RNA probes with their target mRNA molecules in situ while avoiding non-specific binding of probe. Those of skills in the art are capable of adjusting the hybridization conditions (i.e., temperature, concentration of salts and formamide and the like) to specific probes and types of exosomes. Following hybridization, any unbound probe is washed off and the slide is subjected to either a photographic emulsion which reveals signals generated using radio-labeled probes or to a colorimetric reaction which reveals signals generated using enzyme-linked labeled probes.

Oligonucleotide microarray analysis—This method can be performed by attaching oligonucleotide probes which are capable of specifically hybridizing with the transcript of the necroptosis activation pathway (e.g. RIPK1, RIPK3 or MLKL transcript) to a solid surface (e.g., a glass wafer). Each oligonucleotide probe is of approximately 20-25 nucleic acids in length. To detect the expression pattern of the transcript of the necroptosis activation pathway of the present invention in a specific sample (e.g., exosomes), RNA is extracted from the exosomes using methods known in the art (using e.g., a TRIZOL solution, Gibco BRL, USA). Hybridization can take place using either labeled oligonucleotide probes (e.g., 5′-biotinylated probes) or labeled fragments of complementary DNA (cDNA) or RNA (cRNA). Briefly, double stranded cDNA is prepared from the RNA using reverse transcriptase (RT) (e.g., Superscript II RT), DNA ligase and DNA polymerase I, all according to manufacturer's instructions (Invitrogen Life Technologies, Frederick, Md., USA). To prepare labeled cRNA, the double stranded cDNA is subjected to an in vitro transcription reaction in the presence of biotinylated nucleotides using e.g., the BioArray High Yield RNA Transcript Labeling Kit (Enzo, Diagnostics, Affymetix Santa Clara Calif.). For efficient hybridization the labeled cRNA can be fragmented by incubating the RNA in 40 mM Tris Acetate (pH 8.1), 100 mM potassium acetate and 30 mM magnesium acetate for 35 minutes at 94° C. Following hybridization, the microarray is washed and the hybridization signal is scanned using a confocal laser fluorescence scanner which measures fluorescence intensity emitted by the labeled cRNA bound to the probe arrays.

Affymetrix microarray (Affymetrix®, Santa Clara, Calif.)—in this method each gene on the array is represented by a series of different oligonucleotide probes, of which, each probe pair consists of a perfect match oligonucleotide and a mismatch oligonucleotide. While the perfect match probe has a sequence exactly complimentary to the particular gene, thus enabling the measurement of the level of expression of the particular gene, the mismatch probe differs from the perfect match probe by a single base substitution at the center base position. The hybridization signal is scanned using the Agilent scanner, and the Microarray Suite software subtracts the non-specific signal resulting from the mismatch probe from the signal resulting from the perfect match probe.

According to one embodiment, for detection of RIPK1 on the transcript level any of the following commercially bought products can be used: miRTarBase miRNAs that target RIPK1, ViGene Biosciences pre-made micro RNAs for RIPK1 gene (e.g. SH810875), and SwitchGear RIPK1 3′ UTR sequence.

According to one embodiment, for detection of RIPK3 on the transcript level any of the following commercially bought products can be used: ViGene Biosciences pre-made micro RNAs for RIPK3 gene (e.g. SH853021), SwitchGear RIPK3 3′ UTR sequence. According to one embodiment, for detection of MLKL on the transcript level any of the following commercially bought products can be used: ViGene Biosciences pre-made micro RNAs for MLKL gene (e.g. SH806464 or SH877338), SwitchGear MLKL 3′ UTR sequence.

As mentioned, detection of activation of a necroptosis activation pathway in an exosome fraction of a biological sample may be considered positive when there is an increase in the activity or expression of the component of the necroptosis activation pathway in the exosome fraction beyond a predetermined threshold with respect to an activity or expression of the component of the necroptosis activation pathway in an exosome fraction from a non-necroptotic sample.

According to some embodiments, the term “non-necroptotic sample” refers to an unaffected control sample taken from a healthy subject (i.e. known not to have a necroptosis or inflammation) or from the same subject prior to the onset of the necroptosis or inflammation (i.e., healthy). Since biological characteristics depend on, amongst other things, species and age, it is preferable that the control sample is retrieved from a subject of the same species, age and/or gender. Alternatively, control data may be taken from databases and literature. It will be appreciated that the control sample may also be taken from the diseased subject at a particular time-point, in order to analyze the progression (i.e., monitoring) of the disease.

The term “increase” according to specific embodiment should be statistically significant.

According to one embodiment, the increase is by about 5%, about 10%, about 20%, about 30%, about 40%, about 50%, about 60%, about 70%, about 80%, about 90%, about 100% or more.

As mentioned, detection of a biological sample being necroptotic or inflammatory may be considered positive when there is an increase in the exosome level beyond a predetermined threshold with respect to a level of the exosomes in a biological sample from a non-necroptotic sample. Determining the level of exosomes in a sample can be carried out using any method known in the art, as described in detail hereinabove. Once detection of activation of a necroptosis activation pathway is achieved, a method of diagnosing a disease associated with the activation of the necroptosis activation pathway can be attained.

Thus, according to one aspect of the invention, there is provided a method of diagnosing a disease associated with activation of a necroptosis activation pathway in a subject, the method comprising: (a) detecting activation of a necroptosis activation pathway in a biological sample of the subject according to some embodiments of the invention; and (b) diagnosing the subject as having the disease associated with activation of the necroptosis activation pathway when an increase in the activity or expression of a component of the necroptosis activation pathway in the exosome fraction is beyond a predetermined threshold with respect to an activity or expression of the component of the necroptosis activation pathway in an exosome fraction from a non-necroptotic sample.

As used herein, the term “diagnosing” refers to classifying a pathology (e.g., a disease, disorder, syndrome, medical condition and/or a symptom thereof), determining a severity of the pathology, monitoring the progression of a pathology, forecasting an outcome of the pathology and/or prospects of recovery (e.g., prognosis). Diagnosing may also refer to the contribution of ruling out alternative diagnoses.

The phrase “disease associated with activation of a necroptosis activation pathway” as used herein refers to any disease or disorder which involves activation of a component of the necroptosis activation pathway. Exemplary diseases include, but are not limited to, necroptosis, inflammation, necroptosis associated with inflammation, necroptosis associated with an infection, tissue damage, tissue injury, myocardinal infarction (MI), stroke, ischemia-reperfusion injury (IRI), atherosclerosis, psoriasis, rheumatoid diseases (e.g. Rheumatoid arthritis), pancreatitis, diabetes, asthma, emphysema, kidney tissue damage (e.g. Acute tubular necrosis), autoimmune disease (e.g. multiple sclerosis, lupus), inflammatory bowel disease (IBD), Ulcerative colitis (UC), Crohn's disease (CD), neurodegeneration (e.g. Parkinson's disease, Alzheimer's disease), and graft related diseases (e.g. graft rejection and graft versus host disease). Such diseases are discussed in detail below.

According to a specific embodiment the disease is a necroptosis, an inflammation, a necroptosis associated with inflammation, a necroptosis associated with an infection, a brain tissue damage or injury (e.g. neurodegeneration or stroke), a kidney tissue damage or injury (e.g. Acute tubular necrosis), a lung tissue damage or injury (e.g. emphysema), a cardiac tissue damage or injury (e.g. MI) or a gastrointestinal tissue damage or injury (e.g. IBD, UC or CD).

The present teachings also contemplate for a composition of matter comprising exosomoal faction (as described herein) and a reagent for specifically detecting (e.g., primary antibody or oligonucleotide probe, as described hereinabove) for a component in the necroptotic pathway.

Moreover, once detection of a necroptotic or inflammatory sample is achieved, a method of diagnosing a necroptosis or inflammation can be attained.

Thus, according to an alternative or an additional aspect of the invention, there is provided a method of diagnosing necroptosis or inflammation in a subject, the method comprising: (a) detecting a level of exosomes in a biological sample of the subject according to some embodiments of the invention; and (b) diagnosing the subject as having necroptosis or inflammation when an increase in the level of exosomes in the biological sample is beyond a predetermined threshold with respect to a level of the exosomes in a biological sample from a non-necroptotic sample.

The method of diagnosing necroptosis or inflammation may further comprise measuring an activity or expression of a component of a necroptosis activation pathway in the exosomes, wherein a ratio of the activity or expression of the component of the necroptosis activation pathway per level of exosomes (also referred to as normalized expression) beyond a predetermined threshold is indicative of necroptosis or inflammation. The phrase “a ratio of the activity or expression of the component of the necroptosis activation pathway per level of exosomes” refers to the activity or expression of a component of the necroptosis activation pathway per number of exosomes (i.e. in the exosome fraction) of a biological sample.

Once diagnosis is made, the subject may be informed of the disease e.g. a disease associated with activation of a necroptosis activation pathway, a necroptosis or an inflammation.

Diagnosis may be further substantiated with any other method known in the art. For example, necroptosis or inflammation may be corroborated by standard blood tests testing for erythrocyte sedimentation rate (ESR), C-reactive protein (CRP) and plasma viscosity (PV).

The methods of the present invention may be further implemented for assessing a specific tissue undergoing necroptosis e.g., in a subject.

Thus, according to an alternative or an additional aspect, there is provided a method of identifying a tissue undergoing necroptosis, the method comprising: (a) obtaining a biological sample such as from a subject in need thereof; (b) detecting an activity or expression of a component of a necroptosis activation pathway and an expression of a cell specific marker in an exosome fraction of the biological sample; and (c) identifying the tissue undergoing necroptosis based on the measured level of the activity or expression of the component of the necroptosis activation pathway and the expression of the cell specific marker.

According to one embodiment, the tissue is a soft tissue or hard tissue.

The term “cell specific marker” as used herein refers to gene or expression product e.g., mRNA or protein that identify a cell population such as within a heterogeneous cell population. Thus, for example, cell populations can be identified by the presence of human clusters of differentiation (CD) molecules (as exemplified in www.en(dot)wikipedia(dot)org/wiki/List_of human_clusters_of_differentiation, incorporated herein by reference). Additionally or alternatively, different cell populations may be identified by expression of genes or proteins specific for their cellular type, location or function.

As mentioned above, exosomes are derived from various cell types and depending on their cellular origin, comprise biological material from their cells of origin. Thus, the term cell specific marker is meant to include any biological material specific to a cell of origin, such as but not limited to, nucleic acids (e.g. RNA), proteins, peptides, polypeptides and antigens. The proteins may include, for example, membrane expressed proteins such as human clusters of differentiation (CD) molecules, MHC molecules and cellular receptors. RNA may include, for example, mRNA (including coding or non-coding mRNA) as well as miRNA, snRNA, snoRNA, rRNAs, tRNAs, siRNA, hnRNA, or shRNA.

According to one embodiment, each exosome expresses 1, 2, 3, 4, 5, 6, 7, 8, 9, 10, 15, 20, 25, 50, 100, 250, 500, 1000 or more cell specific markers.

According to one embodiment, exosomes are derived from a cell type, including but not limited to, cardiac, pulmonary, pancreas, stomach, intestine, spleen, bladder, nephric, ovarian, testis, uterine, cervical, skin, colon, rectal, colorectal, breast, prostate, brain, head, neck, esophagus, tracheal, hepatic, placenta, lymphoid, mononuclear, bone marrow, or fetal cells.

According to one embodiment, the exosomes are derived from a necroptotic cell, such as a necroptotic cell associated with a disease.

The term “necroptotic cell associated with a disease” refers to a cell undergoing necroptosis as a result (i.e. directly or indirectly) of a disease or condition. It will be appreciated that a disease or condition occurring (i.e. directly or indirectly) as a result of a necroptotic cell is also included under this definition.

According to one embodiment, when the cell is a pulmonary cell, the cell specific marker comprises, for example, a gene or gene product expressed by type I pneumocytes (e.g. aquaporin-5, Aq-5), type II pneumocytes (e.g. surfactants A-D, Sp-A, Sp-B, Sp-C, Sp-D) and clara cells (e.g. clara cell-specific protein, CCSP).

According to one embodiment, when the cell is a prostate cell, the cell specific marker comprises, for example, a gene or gene product of CD10, CD13, CD26, CD38, CD82, CD104 or CD107a/b.

According to one embodiment, when the cell is a hepatic cell, the cell specific marker comprises, for example, a gene or gene product of A-fetoprotein, CK18, CK19, HNF4, albumin, G-6-P or alpha-1-antitrypsin (AAT).

According to one embodiment, when the cell is a pancreatic cell (e.g. pancreatic islet cell such as islets of Langerhans), the cell specific marker comprises, for example, a gene or gene product of insulin, amylase, glucagon, CD142, CD200 or CD318.

According to one embodiment, when the cell is a cell associated with diabetes (e.g. diseased pancreatic β cell), the cell specific marker comprises, for example, a gene or gene product of IL-6, IL-8, CRP, RBP4, CTSS, ITGB2, HLA-DRA, CD53, PLAG27, or MMP9.

According to one embodiment, when the cell is a cardiac cell, the cell specific marker comprises, for example, a gene or gene product of integrin αvβ6, ALCAM (CD166), alpha-Actinin, Annexin 5/6, ANP (atrial natriuretic peptide), Cardiac troponin I (cTnI), Cardiac troponin-T (cTnT), Caveolin-2/3, CNP (C-type natriuretic peptide), CARP (cardiac adriamycin-responsive protein), H-FABP or GATA-4/6.

According to one embodiment, when the cell is a cell associated with a cardiovascular disease (e.g. diseased cardiac cell or blood vessel cell), the cell specific marker comprises, for example, a gene or gene product of FATP6, MRP14 or CD69. Additionally or alternatively, the cell specific marker may comprise one or more overexpressed miRs, such as, but not limited to, miR-195, miR-208, miR-214, let-7b, let-7c, let-7e, miR-15b, miR-23a, miR-24, miR-27a, miR-27b, miR-93, miR-99b, miR-100, miR-103, miR-125b, miR-140, miR-145, miR-181a, miR-191, miR-195, miR-199a, miR-320, miR-342, miR-451, or miR-499, or any combination thereof. Additionally or alternatively, the cell specific marker may comprise one or more underexpressed miRs, such as, but not limited to, miR-1, miR-10a, miR-17-5p, miR-19a, miR-19b, miR-20a, miR-20b, miR-26b, miR-28, miR-30e-5p, miR-101, miR-106a, miR-126, miR-222, miR-374, miR-422b, or miR-423, or any combination thereof.

According to one embodiment, when the cell is a cell associated with stroke (e.g. diseased brain cell or blood vessel cell), the cell specific marker comprises, for example, a gene or gene product of S-100, neuron specific enolase, PARK7, NDKA, ApoC-I, ApoC-III, SAA or AT-III fragment, Lp-PLA2, hs-CRP, MMP9, S100-P, S100A12, S100A9, coagulation factor V, Arginasel, CA-IV, monocarboxylic acid transporter, ets-2, EIF2alpha, cytoskeleton associated protein 4, N-formylpeptide receptor, Ribonuclease2, N-acetylneuraminate pyruvate lyase, BCL-6, or Glycogen phosphorylase.

According to one embodiment, when the cell is a cell associated with Parkinson's Disease (e.g. diseased brain cell or neuron), the cell specific marker comprises, for example, a gene or gene product of PARK2, ceruloplasmin, VDBP, tau, DJ-1, apo-H, Ceruloplasmin, BDNF, IL-8, Beta2-microglobulin, apoAII, ABeta1-42, or DJ-1.

According to one embodiment, when the cell is a cell associated with Alzheimer's Disease (e.g. diseased brain cell or neuron), the cell specific marker comprises, for example, a gene or gene product of APP695, APP751 or APP770, BACE1, cystatin C, amyloid-β, T-τ, complement factor H or α-2-macroglobulin. Additionally or alternatively, the cell specific marker may comprise one or more underexpressed miRs, such as, but not limited to, miR-107, miR-29a, miR-29b-1, or miR-9.

According to one embodiment, when the cell is a cell associated with an autoimmune disease, the cell specific marker comprises, for example, a gene or gene product of Tim-2.

According to one embodiment, when the cell is a cell associated with Irritable Bowel Disease/Inflammatory Bowel Disease (IBD) or Syndrome (IBS) (e.g. diseased colon cell or intestinal cell), the cell specific marker comprises, for example, a gene or gene product of IL-16, IL-1β, IL-12, TNF-α, interferon-γ, IL-6, Rantes, 11-12, MCP-1 or 5HT.

According to one embodiment, when the cell is a cell associated with Ulcerative colitis (UC) or Crohn's disease (CD) (e.g. diseased cell of the gastrointestinal (GI) tract such as a colon cell, rectum cell, or intestinal cell), the cell specific marker comprises, for example, a gene or gene product of IFITM1, IFITM3, STAT1, STAT3, TAP1, PSME2, PSMB8, HNF4G, KLFS, AQP8, APT2B1, SLC16A, MFAP4, CCNG2, SLC44A4, DDAH1, TOB1, MKNK1, CEACAM7*, CDC42SE2, PSD3, GSN, GPM6B, PDPK1, ANP32E, ADAM9, CDH1, NLRP2, OSBPL1, VNN1, RABGAP1L, PHACTR2, ASH1L, CDH1, NLRP2, OSBPL1, VNN1, RABGAP1L, PHACTR2, ASH1, ZNF3, FUT2, IGHA1, EDEM1, GPR171, LOC643187, FLVCR1, ETNK1, LOC728411, POSTN, MUC12, HOXA5, SIGLEC1, LARP5, PIGR, SPTBN1, UFM1, C6orf62, WDR90, ALDH1A3, F2RL1, IGHV1-69, DUOX2, RAB5A, or CP.

According to one embodiment, when the cell is a cell associated with Multiple Sclerosis (MS) (e.g. diseased brain cell or neuroglia cell e.g. oligodendrocyte), the cell specific marker comprises, for example, a gene or gene product of B7, B7-2, CD-95 (fas), Apo-1/Fas, IL-6, IL-17, PAR-3, IL-17, T1/ST2, JunD, 5-LO, LTA4H, MBP, PLP, or alpha-beta crystallin.

According to one embodiment, when the cell is a cell associated with Lupus (e.g. diseased connective tissue cell, cardiac cell, lung cell, kidney cell, brain cell or blood vessel cell), the cell specific marker comprises, for example, a gene or gene product of TNFR.

According to one embodiment, when the cell is a cell associated with asthma (e.g. a diseased cell of the airways, e.g. lung cell), the cell specific marker comprises, for example, a gene or gene product of YKL-40, S-nitrosothiols, SSCA2, PAI, amphiregulin, or periostin.

According to one embodiment, when the cell is a cell associated with psoriasis (e.g. diseased skin cell), the cell specific marker comprises, for example, a gene or gene product of flt-1, VPF receptors, kdr, IL-20, VEGFR-1, VEGFR-2, VEGFR-3, or EGR1. Additionally or alternatively, the cell specific marker may comprise one or more overexpressed miRs, such as, but not limited to, miR-146b, miR-20a, miR-146a, miR-31, miR-200a, miR-17-5p, miR-30e-5p, miR-141, miR-203, miR-142-3p, miR-21, or miR-106a, or any combination thereof.

According to one embodiment, when the cell is a cell associated with a Rheumatic Disease (e.g. diseased joint cell), the cell specific marker comprises, for example, a gene or gene product of citrulinated fibrin α-chain, CD5 antigen-like fibrinogen fragment D, CD5 antigen-like fibrinogen fragment B, TNF-α, HOXD10, HOXD11, HOXD13, CCL8, LIM homeobox2, or CENP-E. Additionally or alternatively the cell specific marker may comprise one or more underexpressed miRs, such as, but not limited to, miR-146a, miR-155, miR-132, miR-16, or miR-181, or any combination thereof.

According to one embodiment, when the cell is a cell associated with Cirrhosis (e.g. diseased liver cell), the cell specific marker comprises, for example, a gene or gene product of NLT, HBsAg, NLT, HBsAG, AST, YKL-40, Hyaluronic acid, TIMP-1, alpha 2 macroglobulin, a-1-antitrypsin PIZ allele, haptoglobin, or acid phosphatase ACP AC.

According to one embodiment, when the cell is a cell associated with HIV (e.g.

diseased T lymphocyte), the cell specific marker comprises, for example, a gene or gene product of gp41 or gp120. Additionally or alternatively the cell specific marker may comprise one or more overexpressed miRs, such as, but not limited to, miR-28, miR-29a, miR-29b, miR-125b, miR-149, miR-150, miR-223, miR-378, miR-324-5p or miR-382.

According to one embodiment, when the cell is a cell associated with sepsis (e.g. blood cell, platelet), the cell specific marker comprises, for example, a gene or gene product of 15-Hydroxy-PG dehydrogenase (up), LAIR1 (up), NFKB1A (up), TLR2, PGLYPR1, TLR4, MD2, TLR5, IFNAR2, IRAK2, IRAK3, IRAK4, PI3K, PI3KCB, MAP2K6, MAPK14, NFKB1A, NFKB1, IL-1R1, MAP2K1IP1, MKNK1, FAS, CASP4, GADD45B, SOCS3, TNFSF10, TNFSF13B, OSM, HGF, or IL-18R1.

According to one embodiment, when the cell is a cell associated with organ rejection, the cell specific marker comprises, for example, a gene or gene product of matix metalloprotein-9, proteinase 3, or HNP. Additionally or alternatively, the cell specific marker may comprise one or more overexpressed miRs, such as, but not limited to, miR-658, miR-125a, miR-320, miR-381, miR-628, miR-602, miR-629, or miR-125a, or any combination thereof. Additionally or alternatively, the cell specific marker may comprise one or more underexpressed miRs, such as, but not limited to, miR-324-3p, miR-611, miR-654, miR-330_MM1, miR-524, miR-17-3p_MM1, miR-483, miR-663, miR-516-5p, miR-326, miR-197_MM2, or miR-346, or any combination thereof.

According to one embodiment, when the cell is a cell associated with kidney failure, the cell specific marker comprises, for example, a gene or gene product of beta-transducin.

According to one embodiment, detecting the expression of a cell specific marker is effected by contacting the biological sample (e.g. exosome fraction of the biological sample) with an agent targeting the cell specific marker and detecting binding between the cell specific marker and the agent using methods such as described hereinabove.

According to another embodiment, detecting an activity or expression of a component of the necroptosis activation pathway is effected by contacting the biological sample (e.g. exosome fraction of the biological sample) with an agent targeting the component of the necroptosis activation pathway and detecting binding between the component of the necroptosis activation pathway and the agent.

An agent according to some embodiments of the invention is typically an affinity binding moiety having a binding affinity (K_{D}) of at least about 2 to about 200 M (i.e. as long as the binding is specific i.e., no background binding), an antibody (e.g. monoclonal antibody, polyclonal antibody, Fabs, Fab′, single chain antibody, synthetic antibody), a DNA, a RNA, an aptamer (DNA/RNA), a peptoid, a zDNA, a peptide nucleic acid (PNA), a locked nucleic acid (LNA), a lectin, a synthetic or naturally occurring chemical compound (including, but not limited to, a drug, a labeling reagent), a dendrimer, or combination of any of these agents.

According to one embodiment, the agent is an antibody.

The term “antibody” as used in this invention includes intact molecules as well as functional fragments thereof, such as Fab, F(ab′)2, and Fv that are capable of binding to macrophages. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule. Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

According to one embodiment, the antibody is coupled to a substrate or linked directly or indirectly to a solid surface and used to identify and optionally isolate the exosome, such as described herein.

According to one embodiment, any of the affinity binding moieties discussed herein can be used in single phase or two phase assays (where the latter typically uses the affinity binding moiety when bound to a solid support/surface).

Accordingly, a solid surface or substrate can be any physically separable solid to which a agent can be directly or indirectly attached including, but not limited to, surfaces provided by microarrays and wells, particles such as beads or microspheres, columns, glass, coated beads or particles, magnetic particles, plastics (including acrylics, polystyrene, copolymers of styrene or other materials, polypropylene, polyethylene, polybutylene, polyurethanes, Teflon™, etc.), polysaccharides, nylon or nitrocellulose, resins, silica or silica-based materials including silicon and modified silicon, carbon, metals, or ceramics.

According to one embodiment, the agent is applied to an exosomal fraction of a biological sample, typically after exosomes are purified or concentrated from the biological sample (as discussed in detail above). Accordingly, exosomes expressing a cell specific marker and/or a component of the necroptosis activation pathway may be identified in a heterogeneous population of exosomes. Alternatively, exosomes expressing a cell specific marker and/or a component of the necroptosis activation pathway may be identified in a homogeneous population of exosomes (i.e. after the exosomal fraction was further purified or concentrated) such as a population of exosomes comprising a particular size or comprising a particular marker profile, as described in detail above.

Alternatively, an agent may be used on a biological sample comprising exosomes without a prior purification step or concentration of exosomes. For example, an agent is used to identify an exosome expressing a cell specific marker and/or a component of the necroptosis activation pathway in a biological sample.

According to one embodiment, a single agent (e.g. antibody) is used to identify an exosome expressing a cell specific marker and/or a component of the necroptosis activation pathway.

According to another embodiment, a combination of different agents may be used to identify an exosome for expression of a cell specific marker and/or a component of the necroptosis activation pathway. For example 2, 3, 4, 5, 6, 7, 8, 9, 10 or more different agents may be used to identify an exosome.

According to a specific embodiment, the agent targeting the component of the necroptosis activation pathway is an antibody (e.g. anti-RIPK1 antibody, anti-RIPK3 or anti-MLKL antibody). According to a specific embodiment, the antibody recognizes the active (i.e. phosphorylated) form of the component of the necroptosis activation pathway (e.g. p-RIPK1, p-RIPK3 or p-MLKL).

Any method known in the art can be used for determination of expression of a cell specific marker and/or a component of the necroptosis activation pathway in exosomes, such as those described above. Exemplary methods include, but are not limited to, MACS, ELISA, and FACS, or any of the methods described hereinabove.

According to one embodiment, the exosomes co-express a component of the necroptosis activation pathway and a cell specific marker.

In order to determine co-expression, the exosomes can be analyzed first for expression of a component of the necroptosis activation pathway, and only the exosomes providing a positive result can then be analyzed for expression of a cell specific marker.

Likewise, co-expression can be determined by first determining expression of a cell specific marker and only the exosomes providing a positive result can then be analyzed for expression of a component of the necroptosis activation pathway.

Alternatively, co-expression can be determined using an agent which recognizes both a component of the necroptosis activation pathway and a cell specific marker. Accordingly, antibodies can be generated using methods well known in the art, for dual targeting.

Identification that a tissue is undergoing necroptosis may be considered positive based on the measured level of the activity or expression of the component of the necroptosis activation pathway and the expression of the cell specific marker.

In order to determine that a tissue is undergoing necroptosis it is sufficient to determine an increase in the activity or expression of a component of the necroptosis activation pathway and an increase in the expression of a cell specific marker in the exosome fraction beyond a predetermined threshold with respect to those in an exosome fraction from a non-necroptotic sample.

According to one embodiment, the increase in the activity or expression of a component of the necroptosis activation pathway in the exosome fraction is by about 5%, about 10%, about 20%, about 30%, about 40%, about 50%, about 60%, about 70%, about 80%, about 90%, about 100% or more with respect to those in an exosome fraction from a non-necroptotic sample.

According to one embodiment, the increase in the expression of a cell specific marker in the exosome fraction is by about 5%, about 10%, about 20%, about 30%, about 40%, about 50%, about 60%, about 70%, about 80%, about 90%, about 100% or more with respect to those in an exosome fraction from a non-necroptotic sample.

Thus, co-expression is not obligatory for determination that a tissue is undergoing necroptosis.

According to another embodiment, in order to determine that a tissue is undergoing necroptosis it is sufficient to determine that 1, 2, 5, 10, 25, 50, 100, 250, 500, 1000, 5000, 10,000, 50,000, 100,000 or more exosomes co-express a component of the necroptosis activation pathway and a cell specific marker.

According to another embodiment, the number of exosomes co-expressing a component of the necroptosis activation pathway and a cell specific marker in the exosomal fraction is about 0.01%, about 0.1%, about 0.5%, about 1%, about 5%, about 10%, about 25% or more.

Following diagnosis of a disease associated with activation of necroptosis activation pathway, a necroptosis or an inflammation, as well as determination that a specific tissue is undergoing necroptosis, the present invention further provides methods of treatment of such diseases.

Thus, according to one aspect of the invention, there is provided a method of treating an inflammation in a subject in need thereof, the method comprising selecting a subject identified as having an inflammation in accordance with the method of some embodiments of the invention, and administering an anti-inflammatory therapy to the subject.

As used herein the term “treating” refers to preventing, curing, reversing, attenuating, alleviating, minimizing, suppressing or halting the deleterious effects of a disease or disorder.

A number of diseases and conditions, which involve an inflammatory response, can be treated using the methodology described hereinabove. Examples of such diseases and conditions are summarized infra.

Inflammatory diseases—Include, but are not limited to, chronic inflammatory diseases and acute inflammatory diseases.

Inflammatory Diseases Associated with Hypersensitivity

Examples of hypersensitivity include, but are not limited to, Type I hypersensitivity, Type II hypersensitivity, Type III hypersensitivity, Type IV hypersensitivity, immediate hypersensitivity, antibody mediated hypersensitivity, immune complex mediated hypersensitivity, T lymphocyte mediated hypersensitivity and DTH.

Type I or immediate hypersensitivity, such as asthma.

Type II hypersensitivity include, but are not limited to, rheumatoid diseases, rheumatoid autoimmune diseases, rheumatoid arthritis (Krenn V. et al., Histol Histopathol 2000 July; 15 (3):791), spondylitis, ankylosing spondylitis (Jan Voswinkel et al., Arthritis Res 2001; 3 (3): 189), systemic diseases, systemic autoimmune diseases, systemic lupus erythematosus (Erikson J. et al., Immunol Res 1998; 17 (1-2):49), sclerosis, systemic sclerosis (Renaudineau Y. et al., Clin Diagn Lab Immunol. 1999 March; 6 (2):156); Chan O T. et al., Immunol Rev 1999 June; 169:107), glandular diseases, glandular autoimmune diseases, pancreatic autoimmune diseases, diabetes, Type I diabetes (Zimmet P. Diabetes Res Clin Pract 1996 October; 34 Suppl:S125), thyroid diseases, autoimmune thyroid diseases, Graves' disease (Orgiazzi J. Endocrinol Metab Clin North Am 2000 June; 29 (2):339), thyroiditis, spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), myxedema, idiopathic myxedema (Mitsuma T. Nippon Rinsho. 1999 August; 57 (8):1759); autoimmune reproductive diseases, ovarian diseases, ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), repeated fetal loss (Tincani A. et al., Lupus 1998; 7 Suppl 2:S107-9), neurodegenerative diseases, neurological diseases, neurological autoimmune diseases, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83), motor neuropathies (Kornberg A J. J Clin Neurosci. 2000 May; 7 (3):191), Guillain-Barre syndrome, neuropathies and autoimmune neuropathies (Kusunoki S. Am J Med Sci. 2000 April; 319 (4):234), myasthenic diseases, Lambert-Eaton myasthenic syndrome (Takamori M. Am J Med Sci. 2000 April; 319 (4):204), paraneoplastic neurological diseases, cerebellar atrophy, paraneoplastic cerebellar atrophy, non-paraneoplastic stiff man syndrome, cerebellar atrophies, progressive cerebellar atrophies, encephalitis, Rasmussen's encephalitis, amyotrophic lateral sclerosis, Sydeham chorea, Gilles de la Tourette syndrome, polyendocrinopathies, autoimmune polyendocrinopathies (Antoine J C. and Honnorat J. Rev Neurol (Paris) 2000 January; 156 (1):23); neuropathies, dysimmune neuropathies (Nobile-Orazio E. et al., Electroencephalogr Clin Neurophysiol Suppl 1999; 50:419); neuromyotonia, acquired neuromyotonia, arthrogryposis multiplex congenita (Vincent A. et al., Ann N Y Acad Sci. 1998 May 13; 841:482), cardiovascular diseases, cardiovascular autoimmune diseases, atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), granulomatosis, Wegener's granulomatosis, arteritis, Takayasu's arteritis and Kawasaki syndrome (Praprotnik S. et al., Wien Klin Wochenschr 2000 Aug. 25; 112 (15-16):660); anti-factor VIII autoimmune disease (Lacroix-Desmazes S. et al., Semin Thromb Hemost.2000; 26 (2):157); vasculitises, necrotizing small vessel vasculitises, microscopic polyangiitis, Churg and Strauss syndrome, glomerulonephritis, pauci-immune focal necrotizing glomerulonephritis, crescentic glomerulonephritis (Noel L H. Ann Med Interne (Paris). 2000 May; 151 (3):178); antiphospholipid syndrome (Flamholz R. et al., J Clin Apheresis 1999; 14 (4):171); heart failure, agonist-like beta-adrenoceptor antibodies in heart failure (Wallukat G. et al., Am J Cardiol. 1999 Jun. 17; 83 (12A):75H), thrombocytopenic purpura (Moccia F. Ann Ital Med Int. 1999 April-June; 14 (2):114); hemolytic anemia, autoimmune hemolytic anemia (Efremov D G. et al., Leuk Lymphoma 1998 January; 28 (3-4):285), gastrointestinal diseases, autoimmune diseases of the gastrointestinal tract, intestinal diseases, chronic inflammatory intestinal disease (Garcia Herola A. et al., Gastroenterol Hepatol. 2000 January; 23 (1):16), celiac disease (Landau Y E. and Shoenfeld Y. Harefuah 2000 Jan. 16; 138 (2):122), autoimmune diseases of the musculature, myositis, autoimmune myositis, Sjogren's syndrome (Feist E. et al., Int Arch Allergy Immunol 2000 September; 123 (1):92); smooth muscle autoimmune disease (Zauli D. et al., Biomed Pharmacother 1999 June; 53 (5-6):234), hepatic diseases, hepatic autoimmune diseases, autoimmune hepatitis (Manns M P. J Hepatol 2000 August; 33 (2):326) and primary biliary cirrhosis (Strassburg C P. et al., Eur J Gastroenterol Hepatol. 1999 June; 11 (6):595).

Type IV or T cell mediated hypersensitivity, include, but are not limited to, rheumatoid diseases, rheumatoid arthritis (Tisch R, McDevitt H O. Proc Natl Acad Sci USA 1994 Jan. 18; 91 (2):437), systemic diseases, systemic autoimmune diseases, systemic lupus erythematosus (Datta S K., Lupus 1998; 7 (9):591), glandular diseases, glandular autoimmune diseases, pancreatic diseases, pancreatic autoimmune diseases, Type 1 diabetes (Castano L. and Eisenbarth G S. Ann. Rev. Immunol. 8:647); thyroid diseases, autoimmune thyroid diseases, Graves' disease (Sakata S. et al., Mol Cell Endocrinol 1993 March; 92 (1):77); ovarian diseases (Garza K M. et al., J Reprod Immunol 1998 February; 37 (2):87), prostatitis, autoimmune prostatitis (Alexander R B. et al., Urology 1997 December; 50 (6):893), polyglandular syndrome, autoimmune polyglandular syndrome, Type I autoimmune polyglandular syndrome (Hara T. et al., Blood. 1991 Mar. 1; 77 (5):1127), neurological diseases, autoimmune neurological diseases, multiple sclerosis, neuritis, optic neuritis (Soderstrom M. et al., J Neurol Neurosurg Psychiatry 1994 May; 57 (5):544), myasthenia gravis (Oshima M. et al., Eur J Immunol 1990 December; 20 (12):2563), stiff-man syndrome (Hiemstra H S. et al., Proc Natl Acad Sci USA 2001 Mar. 27; 98 (7):3988), cardiovascular diseases, cardiac autoimmunity in Chagas' disease (Cunha-Neto E. et al., J Clin Invest 1996 Oct. 15; 98 (8):1709), autoimmune thrombocytopenic purpura (Semple J W. et al., Blood 1996 May 15; 87 (10):4245), anti-helper T lymphocyte autoimmunity (Caporossi A P. et al., Viral Immunol 1998; 11 (1):9), hemolytic anemia (Sallah S. et al., Ann Hematol 1997 March; 74 (3):139), hepatic diseases, hepatic autoimmune diseases, hepatitis, chronic active hepatitis (Franco A. et al., Clin Immunol Immunopathol 1990 March; 54 (3):382), biliary cirrhosis, primary biliary cirrhosis (Jones D E. Clin Sci (Colch) 1996 November; 91 (5):551), nephric diseases, nephric autoimmune diseases, nephritis, interstitial nephritis (Kelly C J. J Am Soc Nephrol 1990 August; 1 (2):140), connective tissue diseases, ear diseases, autoimmune connective tissue diseases, autoimmune ear disease (Yoo T J. et al., Cell Immunol 1994 August; 157 (1):249), disease of the inner ear (Gloddek B. et al., Ann N Y Acad Sci 1997 Dec. 29; 830:266), skin diseases, cutaneous diseases, dermal diseases, bullous skin diseases, pemphigus vulgaris, bullous pemphigoid and pemphigus foliaceus.

Examples of delayed type hypersensitivity include, but are not limited to, contact dermatitis and drug eruption.

Examples of types of T lymphocyte mediating hypersensitivity include, but are not limited to, helper T lymphocytes and cytotoxic T lymphocytes.

Examples of helper T lymphocyte-mediated hypersensitivity include, but are not limited to, Thl lymphocyte mediated hypersensitivity and Th2 lymphocyte mediated hypersensitivity.

Autoimmune Diseases

Include, but are not limited to, cardiovascular diseases, rheumatoid diseases, glandular diseases, gastrointestinal diseases, cutaneous diseases, hepatic diseases, neurological diseases, muscular diseases, nephric diseases, diseases related to reproduction, connective tissue diseases and systemic diseases.

Examples of autoimmune cardiovascular diseases include, but are not limited to atherosclerosis (Matsuura E. et al., Lupus. 1998; 7 Suppl 2:S135), myocardial infarction (Vaarala O. Lupus. 1998;7 Suppl 2:S132), thrombosis (Tincani A. et al., Lupus 1998; 7 Suppl 2:S107-9), Wegener's granulomatosis, Takayasu's arteritis, Kawasaki syndrome (Praprotnik S. et al., Wien Klin Wochenschr 2000 Aug. 25; 112 (15-16):660), anti-factor VIII autoimmune disease (Lacroix-Desmazes S. et al., Semin Thromb Hemost.2000; 26 (2):157), necrotizing small vessel vasculitis, microscopic polyangiitis, Churg and Strauss syndrome, pauci-immune focal necrotizing and crescentic glomerulonephritis (Noel L H. Ann Med Interne (Paris). 2000 May; 151 (3):178), antiphospholipid syndrome (Flamholz R. et al., J Clin Apheresis 1999; 14 (4):171), antibody-induced heart failure (Wallukat G. et al., Am J Cardiol. 1999 Jun. 17; 83 (12A):75H), thrombocytopenic purpura (Moccia F. Ann Ital Med Int. 1999 April-June; 14 (2):114; Semple J W. et al., Blood 1996 May 15; 87 (10):4245), autoimmune hemolytic anemia (Efremov D G. et al., Leuk Lymphoma 1998 January; 28 (3-4):285; Sallah S. et al., Ann Hematol 1997 March; 74 (3):139), cardiac autoimmunity in Chagas' disease (Cunha-Neto E. et al., J Clin Invest 1996 Oct. 15; 98 (8):1709) and anti-helper T lymphocyte autoimmunity (Caporossi A P. et al., Viral Immunol 1998; 11 (1):9).

Examples of autoimmune rheumatoid diseases include, but are not limited to rheumatoid arthritis (Krenn V. et al., Histol Histopathol 2000 July; 15 (3):791; Tisch R, McDevitt H O. Proc Natl Acad Sci USA 1994 Jan. 18; 91 (2):437) and ankylosing spondylitis (Jan Voswinkel et al., Arthritis Res 2001; 3 (3): 189).

Examples of autoimmune glandular diseases include, but are not limited to, pancreatic disease, Type I diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome. Diseases include, but are not limited to autoimmune diseases of the pancreas, Type 1 diabetes (Castano L. and Eisenbarth G S. Ann. Rev. Immunol. 8:647; Zimmet P. Diabetes Res Clin Pract 1996 October; 34 Suppl:S125), autoimmune thyroid diseases, Graves' disease (Orgiazzi J. Endocrinol Metab Clin North Am 2000 June; 29 (2):339; Sakata S. et al., Mol Cell Endocrinol 1993 March; 92 (1):77), spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), idiopathic myxedema (Mitsuma T. Nippon Rinsho. 1999 August; 57 (8):1759), ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), autoimmune prostatitis (Alexander R B. et al., Urology 1997 December; 50 (6):893) and Type I autoimmune polyglandular syndrome (Hara T. et al., Blood. 1991 Mar. 1; 77 (5):1127).

Examples of autoimmune gastrointestinal diseases include, but are not limited to, chronic inflammatory intestinal diseases (Garcia Herola A. et al., Gastroenterol Hepatol. 2000 January; 23 (1):16), celiac disease (Landau Y E. and Shoenfeld Y. Harefuah 2000 Jan. 16; 138 (2):122), colitis, ileitis and Crohn's disease.

Examples of autoimmune cutaneous diseases include, but are not limited to, autoimmune bullous skin diseases, such as, but are not limited to, pemphigus vulgaris, bullous pemphigoid and pemphigus foliaceus.

Examples of autoimmune hepatic diseases include, but are not limited to, hepatitis, autoimmune chronic active hepatitis (Franco A. et al., Clin Immunol Immunopathol 1990 March; 54 (3):382), primary biliary cirrhosis (Jones D E. Clin Sci (Colch) 1996 November; 91 (5):551; Strassburg C P. et al., Eur J Gastroenterol Hepatol. 1999 June; 11 (6):595) and autoimmune hepatitis (Manns M P. J Hepatol 2000 August; 33 (2):326).

Examples of autoimmune neurological diseases include, but are not limited to, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83; Oshima M. et al., Eur J Immunol 1990 December; 20 (12):2563), neuropathies, motor neuropathies (Kornberg A J. J Clin Neurosci. 2000 May; 7 (3):191); Guillain-Barre syndrome and autoimmune neuropathies (Kusunoki S. Am J Med Sci. 2000 April; 319 (4):234), myasthenia, Lambert-Eaton myasthenic syndrome (Takamori M. Am J Med Sci. 2000 April; 319 (4):204); paraneoplastic neurological diseases, cerebellar atrophy, paraneoplastic cerebellar atrophy and stiff-man syndrome (Hiemstra H S. et al., Proc Natl Acad Sci USA 2001 Mar. 27; 98 (7):3988); non-paraneoplastic stiff man syndrome, progressive cerebellar atrophies, encephalitis, Rasmussen's encephalitis, amyotrophic lateral sclerosis, Sydeham chorea, Gilles de la Tourette syndrome and autoimmune polyendocrinopathies (Antoine J C. and Honnorat J. Rev Neurol (Paris) 2000 January; 156 (1):23); dysimmune neuropathies (Nobile-Orazio E. et al., Electroencephalogr Clin Neurophysiol Suppl 1999; 50:419); acquired neuromyotonia, arthrogryposis multiplex congenita (Vincent A. et al., Ann N Y Acad Sci. 1998 May 13; 841:482), neuritis, optic neuritis (Soderstrom M. et al., J Neurol Neurosurg Psychiatry 1994 May; 57 (5):544) and neurodegenerative diseases.

Examples of autoimmune muscular diseases include, but are not limited to, myositis, autoimmune myositis and primary Sjogren's syndrome (Feist E. et al., Int Arch Allergy Immunol 2000 September; 123 (1):92) and smooth muscle autoimmune disease (Zauli D. et al., Biomed Pharmacother 1999 June; 53 (5-6):234).

Examples of autoimmune nephric diseases include, but are not limited to, nephritis and autoimmune interstitial nephritis (Kelly C J. J Am Soc Nephrol 1990 August; 1 (2):140).

Examples of autoimmune diseases related to reproduction include, but are not limited to, repeated fetal loss (Tincani A. et al., Lupus 1998; 7 Suppl 2:S107-9).

Examples of autoimmune connective tissue diseases include, but are not limited to, ear diseases, autoimmune ear diseases (Yoo T J. et al., Cell Immunol 1994 August; 157 (1):249) and autoimmune diseases of the inner ear (Gloddek B. et al., Ann N Y Acad Sci 1997 Dec. 29; 830:266).

Examples of autoimmune systemic diseases include, but are not limited to, systemic lupus erythematosus (Erikson J. et al., Immunol Res 1998; 17 (1-2):49) and systemic sclerosis (Renaudineau Y. et al., Clin Diagn Lab Immunol. 1999 March; 6 (2):156); Chan O T. et al., Immunol Rev 1999 June; 169:107).

Infectious Diseases

Examples of infectious diseases include, but are not limited to, chronic infectious diseases, subacute infectious diseases, acute infectious diseases, viral diseases, bacterial diseases, protozoan diseases, parasitic diseases, fungal diseases, mycoplasma diseases and prion diseases.

Graft Rejection Diseases

Examples of diseases associated with transplantation of a graft include, but are not limited to, graft rejection, chronic graft rejection, subacute graft rejection, hyper-acute graft rejection, acute graft rejection and graft versus host disease (GVHD).

Allergic Diseases

Examples of allergic diseases include, but are not limited to, asthma, hives, urticaria, pollen allergy, dust mite allergy, venom allergy, cosmetics allergy, latex allergy, chemical allergy, drug allergy, insect bite allergy, animal dander allergy, stinging plant allergy, poison ivy allergy and food allergy.

In a specific embodiment, the inflammatory condition is associated with an infectious disease, an autoimmune disease, a hypersensitivity associated inflammation, a graft rejection, a tissue injury or damage.

The methods of the invention may be used to treat any injury or damage to a cell, tissue (e.g. soft tissue) or organ, including, but not limited to, acute, chronic, ischemic, or traumatic (e.g. such as that associated with a surgery or accident) injury to the skeletal muscle, heart (e.g. cardiac muscle or cardiovascular cell), kidney, liver, intestine, brain, lung, pancreas, vascular, dermal tissue, scalp, or eye as well as ischemia-reperfusion injury (IRI).

As mentioned hereinabove, the method of this aspect of the present invention is affected by administering to the subject an anti-inflammatory therapy. Thus, for example, the anti-inflammatory therapy may include, without being limited to, NSAIDs (Non-Steroidal Anti-inflammatory Drugs), corticosteroids (such as prednisone) and anti-histamines.

Anti-inflammatory agents which may be used according to the present teachings include, but are not limited to, Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Momiflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; Zomepirac Sodium.

Any of the above described agents may be administered individually or in combination.

According to an alternative or an additional aspect of the invention, there is provided a method of treating necroptosis in a subject in need thereof, the method comprising selecting a subject identified as having a necroptosis in accordance with the method of some embodiments of the invention, and administering an anti-necroptosis therapy to the subject.

A number of diseases and conditions which involve necroptosis can be treated using the present methodology. Exemplary diseases include, but are not limited to, inflammation, tissue injury or damage, infectious diseases, sepsis, myocardinal infarction, stroke, organ ischemia, ischemia-reperfusion injury (IRI), atherosclerosis, psoriasis, rheumatoid diseases, pancreatitis (e.g. acute necrotizing pancreatitis), liver diseases (e.g. Cirrhosis, Hepatitis), diabetes, asthma, autoimmune diseases (e.g. multiple sclerosis, lupus), inflammatory bowel disease (IBD), Ulcerative colitis (UC), Crohn's disease (CD), brain injury (e.g. traumatic brain injury), neurodegeneration (e.g. Parkinson's disease, Alzheimer's disease), cancer chemo/radiation therapy-induced necroptosis, thyroiditis and graft related diseases (e.g. organ/graft rejection and graft versus host disease).

As mentioned above, the methods of the invention may be used to treat any injury or damage to an organ or tissue, such as an organ or tissue (e.g. brain, heart, lung, kidney, liver, intestine, spleen or pancreas) undergoing necroptosis.

According to one embodiment, the necroptosis is associated with inflammation.

According to one embodiment, the necroptosis is associated with a tissue injury or damage.

According to a specific embodiment, the injury is an acute organ injury.

According to a specific embodiment, the injury is a chronic organ injury.

As mentioned hereinabove, the method of this aspect of the present invention is affected by administering to the subject an anti-necroptosis therapy. Thus, for example, the anti-necroptosis therapy may include, without being limited to, an anti-inflammatory agent, an immunosuppressant agent, non-steroid anti-inflammatory drugs (NSAIDs) or a small molecule inhibitor of necroptosis.

Exemplary anti-inflammatory agents are described in detail above.

Examples of immunosuppressive agents include, but are not limited to, Tacrolimus (also referred to as FK-506 or fujimycin, trade names: Prograf, Advagraf, Protopic), Mycophenolate Mofetil, Mycophenolate Sodium, Prednisone, methotrexate, cyclophosphamide, cyclosporine, cyclosporin A, chloroquine, hydroxychloroquine, sulfasalazine (sulphasalazopyrine), gold salts, D-penicillamine, leflunomide, azathioprine, anakinra, infliximab (REMICADE), etanercept, TNF.alpha. blockers, a biological agent that targets an inflammatory cytokine, and Non-Steroidal Anti-Inflammatory Drug (NSAIDs). Examples of NSAIDs include, but are not limited to aspirin, acetyl salicylic acid, choline magnesium salicylate, diflunisal, magnesium salicylate, salsalate, sodium salicylate, diclofenac, etodolac, fenoprofen, flurbiprofen, indomethacin, ketoprofen, ketorolac, meclofenamate, naproxen, nabumetone, phenylbutazone, piroxicam, sulindac, tolmetin, acetaminophen, ibuprofen, Cox-2 inhibitors, tramadol, rapamycin (sirolimus) and rapamycin analogs (such as CCI-779, RAD001, AP23573).

Ample guidance for selecting and administering suitable immunosuppressive agents are provided in the literature (for example, refer to: Kirkpatrick C H. and Rowlands D T Jr., 1992. JAMA. 268, 2952; Higgins R M. et al., 1996. Lancet 348, 1208; Suthanthiran M. and Strom T B., 1996. New Engl. J. Med. 331, 365; Midthun D E. et al., 1997. Mayo Clin Proc. 72, 175; Morrison V A. et al., 1994. Am J Med. 97, 14; Hanto D W., 1995. Annu Rev Med. 46, 381; Senderowicz A M. et al., 1997. Ann Intern Med. 126, 882; Vincenti F. et al., 1998. New Engl. J. Med. 338, 161; Dantal J. et al. 1998. Lancet 351, 623).

Additionally, necroptosis can be treated using any small molecule inhibitor of necroptosis e.g. heterocyclic derivatives that inhibit TNF-α induced necroptosis, as taught e.g. in U.S. Pat. Nos. 9,108,955 and 8,278,344, and in U.S. Patent Application Nos. 20140024657, 20160024098, 20140323489, 20160102053, 20140128437, 20120122889, 20120309795, 20100317701 and 20090099242, all of which are incorporated herein by reference.

According to one embodiment, the anti-necroptosis therapy comprises an agent for downregulating an activity or expression of at least one of MLKL, RIPK1, RIPK3, TNF-α or a Toll-like receptor ligand.

According to one embodiment, the agent for downregulating an activity or expression of MLKL comprises necrosulfonamide (see Sun et al., Cell (2012) 148, 213-227). According to another embodiment, the agent for downregulating an activity or expression of MLKL comprises a RNAi, a shRNA, or a siRNA, as discussed in further detail below.

According to a specific embodiment, the agent for downregulating the activity or expression of MLKL specifically compromises the necroptotic activity of MLKL without compromising an endocytic activity of MLKL.

Screening for agents capable of compromising the necroptotic activity of MLKL without compromising an endocytic activity of MLKL can be carried out using any method known in the art and as described in detail hereinbelow.

According to one embodiment, the agent for downregulating an activity or expression of RIPK1 or RIPK3 comprises a small organic molecule, a RNAi, a shRNA, or a siRNA, as discussed in further detail below.

According to one embodiment, the agent for downregulating an activity or expression of RIPK1 comprises Necrostatin-1 (commercially available from e.g. ApexBio Technology).

According to one embodiment, the agent for downregulating an activity or expression of RIPK3 comprises GSK' 872 (commercially available from e.g. Merck Millipore).

According to one embodiment, the agent for downregulating an activity or expression of TNF-α comprises, for example, a Tumor Necrosis Factor (TNF) Blocker marketed as e.g. Remicade, Enbrel, Humira, Cimzia, and Simponi.

According to one embodiment, the agent for downregulating an activity or expression of Toll-like receptor ligand comprises, for example, a neutralizing antibody or small molecule antagonist. Exemplary drugs targeting TLRs are taught in Savva A. and Roger T, Front Immunol. (2013) 4: 387, incorporated herein by reference.

As mentioned above, the present inventors have illustrated that MLKL is involved in the general endocytic/exocytic pathways of cells. Indeed the present inventors have uncovered a role of MLKL in regulation of endosomal trafficking. Specifically, MLKL facilitates the transport of endocytosed proteins to intraluminal vesicles (ILVs) within the multivesicular bodies (MVBs) (see Examples 4 and 5 of the Examples section which follows). Furthermore, the present inventors illustrated that deficiency of MLKL results in marked reduction in ILV generation, slowdown of lysosomal degradation of endocytosed proteins, and in a marked potentiation of extracellular ligands (e.g. TNF) (see Example 7 of the Examples section which follows).

Thus, according to an alternative or an additional aspect of the invention there is provided a method of modulating endocytosis of a cell surface receptor capable of ligand induced endocytosis, the method comprising contacting a cell which expresses the cell surface receptor with an agent capable of downregulating an activity or expression of a MLKL, thereby modulating endocytosis of the cell surface receptor.

The cell surface receptor of some embodiments of the invention is capable of ligand induced endocytosis. Exemplary cell surface receptors include, but are not limited to, tumor necrosis factor receptor family (TNFR, including but not limited to TNFR-1, TNFR-2, OX40, CD40, CD27, CD30, Fas receptor, 4-1BB, RANK, TROY, Death receptor-3, Death receptor-4, Death receptor-5, Death receptor-6), transforming growth factor beta receptor (TGF-β receptor, including but not limited to, TGFβR1 (ALK5), TGFβR2, TGFβR3 (β-glycan)), epidermal growth factor receptor family (EGFR, including, but not limited to, EGFR (ErbB-1), HER2/c-neu (ErbB-2), Her 3 (ErbB-3) and Her 4 (ErbB-4)), vascular endothelial growth factor receptor (VEGFR including, but not limited to, VEGFR-1, VEGFR-2, and VEGFR-3), fibroblast growth factor receptor FGF receptor (FGFR, including, but not limited to, FGFR1, FGFR2, FGFR3, FGFR4), Type I cytokine receptor (including, but not limited to, IL-2R, IL-4R, IL-7R, IL-9R, IL-13R, IL-15R, GM-CSF receptor, IL-3R, IL-5R, Leukemia inhibitory factor receptor (LIFR)), Type II cytokine receptor (including, but not limited to, interferon receptor e.g. interferon-alpha/beta receptor, interferon-gamma receptor; and interleukin receptor e.g. IL-10R, IL-20R, IL-22R, IL-28R), Immunoglobulin (Ig) receptor superfamily (including, but not limited to, antigen receptor, MHC I, MHC II, beta-2 microglobulin, CD4, CD8, CD19, CD3, CD79, CD28, CD80, CD86, killer-cell immunoglobulin-like receptors (KIR), leukocyte immunoglobulin-like receptors (LILR), IL-6R, platelet-derived growth factor receptor (PDGFR)), chemokine receptor (e.g. CXCR4 and CCR5), insulin receptor and LDL receptor.

The term “ligand” as used herein refers to a naturally occurring or synthetic compound that binds to a cell surface receptor. The ligand of some embodiments of the invention may comprise, for example, a polypeptide, a protein, a metabolite, a hormone or a nucleic acid (e.g. double stranded DNA).

Exemplary ligands include, without being limited to, antigens (e.g. viral, bacterial, tumor associated), cytokines including lymphokines, interleukins, and chemokines such as, but not limited to, IL-2, IL-3, IL-4, IL-5, IL-7, IL-9, IL-10, IL-12, IL-10, IL-13, IL-15, IL-18, IL-20, IL-21, IL-22, IL-23, IL-28, GM-CSF, Leukemia inhibitory factor (LIF), IFN-γ, transforming growth factor beta (TGF-β), tumor necrosis factor (TNF) family members (including, but not limited to, TNFα, Lymphotoxin-alpha (LT-alpha), heterotrimers of lymphotoxin-beta (LT-beta) and LT-alpha, CD40L, CD27L, CD30L, OX40L, CD154, FasL, CD70, CD153, 4-1BB ligand, TRAIL, RANKL, TWEAK, BAFF, LIGHT, NT-3, NT-4, NGF, TL-1A, EDA-A2), chemokines including C-C chemokines (e.g. RANTES, MCP-1, MIP-1α, and MIP-1B), C-X-C chemokines (e.g. IL-8), C chemokines (e.g. Lymphotactin), and CXXXC chemokines (e.g. Fractalkine), neurotransmitters, glucagon, insulin and other growth factors (including, but not limited to, thrombopoietin (TPO), Erythropoietin (EPO), Fibroblast growth factor (FGF), ephrin, macrophage colony-stimulating factor (m-CSF), Granulocyte colony-stimulating factor (G-CSF), Granulocyte macrophage colony-stimulating factor (GM-CSF), Epidermal growth factor (EGF), vascular endothelial growth factor (VEGF)).

According to a specific embodiment, the ligand is a TNF family member including, but not limited to, TNFα, Lymphotoxin-alpha (LT-alpha), heterotrimers of lymphotoxin-beta (LT-beta) and LT-alpha, CD40L, CD27L, CD30L, OX40L, CD154, FasL, CD70, CD153, 4-1BB ligand, TRAIL, RANKL, TWEAK, BAFF, LIGHT, NT-3, NT-4, NGF, TL-1A, EDA-A2.

The phrase “ligand induced endocytosis “refers to a process by which a ligand binds to a cell surface receptor (e.g. on the surface of the cell membrane) and the resulting ligand-cell surface receptor complex is internalized by the cell, i.e., moves into the cytoplasm of the cell or a compartment within the cytoplasm of the cell (endosomes, vesicles etc.) without causing irreparable damage to the cell membrane. Internalization may be followed up by dissociation of the resulting complex within the cytoplasm and typically results in receptor degradation.

Exemplary cells include, but are not limited to, cardiac, spleen, breast, lung, head, neck, prostate, esophageal, tracheal, brain, liver, bladder, stomach, pancreatic, ovarian, uterine, cervical, testicular, colon, rectal, kidney and skin cells.

Downregulation of MLKL can be affected directly (i.e. by downregulating an activity or expression of MLKL) or by downregulation of a factor which activates MLKL (e.g. by downregulating the activity or expression of RIPK1 or RIPK3).

As used herein the phrase “dowregulating an activity or expression” refers to dowregulating the expression of a protein (e.g. MLKL, RIPK3 or RIPK1) at the genomic (e.g. homologous recombination and site specific endonucleases) and/or the transcript level using a variety of molecules which interfere with transcription and/or translation (e.g., RNA silencing agents) or on the protein level (e.g., aptamers, small molecules and inhibitory peptides, antagonists, enzymes that cleave the polypeptide, antibodies and the like).

For the same culture conditions the activity or expression is generally expressed in comparison to the expression in a cell of the same species but not contacted with the agent or contacted with a vehicle control, also referred to as control.

Downregulation of an activity or expression may be either transient or permanent.

According to specific embodiments, downregulating expression refers to the absence of mRNA and/or protein, as detected by RT-PCR or Western blot, respectively.

According to other specific embodiments downregulating expression refers to a decrease in the level of mRNA and/or protein, as detected by RT-PCR or Western blot, respectively. The reduction may be by at least a 10%, at least 20%, at least 30%, at least 40%, at least 50%, at least 60%, at least 70%, at least 80%, at least 90%, at least 95% or at least 99% reduction.

Non-limiting examples of agents capable of down regulating MLKL, RIPK3 or RIPK1 activity or expression are described in details hereinbelow.

Down-Regulation at the Nucleic Acid Level

Down-regulation at the nucleic acid level is typically effected using a nucleic acid agent, having a nucleic acid backbone, DNA, RNA, mimetics thereof or a combination of same. The nucleic acid agent may be encoded from a DNA molecule or provided to the cell per se.

Thus, downregulation of MLKL, RIPK3 or RIPK1 can be achieved by RNA silencing. As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of specifically inhibiting or “silencing” the expression of a target gene. In certain embodiments, the RNA silencing agent is capable of preventing complete processing (e.g, the full translation and/or expression) of an mRNA molecule through a post-transcriptional silencing mechanism. RNA silencing agents include non-coding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs.

In one embodiment, the RNA silencing agent is capable of inducing RNA interference.

In another embodiment, the RNA silencing agent is capable of mediating translational repression.

According to an embodiment of the invention, the RNA silencing agent is specific to the target RNA (e.g., MLKL, RIPK3 or RIPK1) and does not cross inhibit or silence other targets or a splice variant which exhibits 99% or less global homology to the target gene, e.g., less than 98%, 97%, 96%, 95%, 94%, 93%, 92%, 91%, 90%, 89%, 88%, 87%, 86%, 85%, 84%, 83%, 82%, 81% global homology to the target gene; as determined by PCR, Western blot, Immunohistochemistry and/or flow cytometry.

RNA interference refers to the process of sequence-specific post-transcriptional gene silencing in animals mediated by short interfering RNAs (siRNAs).

Following is a detailed description on RNA silencing agents that can be used according to specific embodiments of the present invention.

DsRNA, siRNA and shRNA—The presence of long dsRNAs in cells stimulates the activity of a ribonuclease III enzyme referred to as dicer. Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex.

Accordingly, some embodiments of the invention contemplate use of dsRNA to downregulate protein expression from mRNA.

According to one embodiment dsRNA longer than 30 bp are used. Various studies demonstrate that long dsRNAs can be used to silence gene expression without inducing the stress response or causing significant off-target effects—see for example [Strat et al., Nucleic Acids Research, 2006, Vol. 34, No. 13 3803-3810; Bhargava A et al. Brain Res. Protoc. 2004; 13:115-125; Diallo M., et al., Oligonucleotides. 2003; 13:381-392; Paddison P. J., et al., Proc. Natl Acad. Sci. USA. 2002; 99:1443-1448; Tran N., et al., FEBS Lett. 2004; 573:127-134].

According to some embodiments of the invention, dsRNA is provided in cells where the interferon pathway is not activated, see for example Billy et al., PNAS 2001, Vol 98, pages 14428-14433. and Diallo et al, Oligonucleotides, Oct. 1, 2003, 13(5): 381-392. doi: 10.1089/154545703322617069.

According to an embodiment of the invention, the long dsRNA are specifically designed not to induce the interferon and PKR pathways for down-regulating gene expression. For example, Shinagwa and Ishii [Genes & Dev. 17 (11): 1340-1345, 2003] have developed a vector, named pDECAP, to express long double-strand RNA from an RNA polymerase II (Pol II) promoter. Because the transcripts from pDECAP lack both the 5′-cap structure and the 3′-poly(A) tail that facilitate ds-RNA export to the cytoplasm, long ds-RNA from pDECAP does not induce the interferon response.

Another method of evading the interferon and PKR pathways in mammalian systems is by introduction of small inhibitory RNAs (siRNAs) either via transfection or endogenous expression.

The term “siRNA” refers to small inhibitory RNA duplexes (generally between 18-30 base pairs) that induce the RNA interference (RNAi) pathway. Typically, siRNAs are chemically synthesized as 2lmers with a central 19 bp duplex region and symmetric 2-base 3′-overhangs on the termini, although it has been recently described that chemically synthesized RNA duplexes of 25-30 base length can have as much as a 100-fold increase in potency compared with 21 mers at the same location. The observed increased potency obtained using longer RNAs in triggering RNAi is suggested to result from providing Dicer with a substrate (27 mer) instead of a product (21 mer) and that this improves the rate or efficiency of entry of the siRNA duplex into RISC.

It has been found that position of the 3′-overhang influences potency of an siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

The strands of a double-stranded interfering RNA (e.g., an siRNA) may be connected to form a hairpin or stem-loop structure (e.g., an shRNA). Thus, as mentioned, the RNA silencing agent of some embodiments of the invention may also be a short hairpin RNA (shRNA).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Examples of oligonucleotide sequences that can be used to form the loop include 5′-CAAGAGA-3′ and 5′-UUACAA-3′ (International Patent Application Nos. WO2013126963 and WO2014107763). It will be recognized by one of skill in the art that the resulting single chain oligonucleotide forms a stem-loop or hairpin structure comprising a double-stranded region capable of interacting with the RNAi machinery.

Synthesis of RNA silencing agents suitable for use with some embodiments of the invention can be effected as follows. First, the MLKL, RIPK3 or RIPK1 mRNA sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (www.ambion.com/techlib/tn/91/912.html).

Second, potential target sites are compared to an appropriate genomic database (e.g., human, mouse, rat etc.) using any sequence alignment software, such as the BLAST software available from the NCBI server (www.ncbi.nlm.nih.gov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

It will be appreciated that, and as mentioned hereinabove, the RNA silencing agent of some embodiments of the invention need not be limited to those molecules containing only RNA, but further encompasses chemically-modified nucleotides and non-nucleotides.

miRNA and miRNA mimics—According to another embodiment the RNA silencing agent may be a miRNA.

The term “microRNA”, “miRNA”, and “miR” are synonymous and refer to a collection of non-coding single-stranded RNA molecules of about 19-28 nucleotides in length, which regulate gene expression. miRNAs are found in a wide range of organisms (viruses.fwdarw.humans) and have been shown to play a role in development, homeostasis, and disease etiology.

Below is a brief description of the mechanism of miRNA activity. Genes coding for miRNAs are transcribed leading to production of a miRNA precursor known as the pri-miRNA. The pri-miRNA is typically part of a polycistronic RNA comprising multiple pri-miRNAs. The pri-miRNA may form a hairpin with a stem and loop. The stem may comprise mismatched bases.

The hairpin structure of the pri-miRNA is recognized by Drosha, which is an RNase III endonuclease. Drosha typically recognizes terminal loops in the pri-miRNA and cleaves approximately two helical turns into the stem to produce a 60-70 nucleotide precursor known as the pre-miRNA. Drosha cleaves the pri-miRNA with a staggered cut typical of RNase III endonucleases yielding a pre-miRNA stem loop with a 5′ phosphate and ˜2 nucleotide 3′ overhang. It is estimated that approximately one helical turn of stem (˜10 nucleotides) extending beyond the Drosha cleavage site is essential for efficient processing. The pre-miRNA is then actively transported from the nucleus to the cytoplasm by Ran-GTP and the export receptor Ex-portin-5.

The double-stranded stem of the pre-miRNA is then recognized by Dicer, which is also an RNase III endonuclease. Dicer may also recognize the 5′ phosphate and 3′ overhang at the base of the stem loop. Dicer then cleaves off the terminal loop two helical turns away from the base of the stem loop leaving an additional 5′ phosphate and ˜2 nucleotide 3′ overhang. The resulting siRNA-like duplex, which may comprise mismatches, comprises the mature miRNA and a similar-sized fragment known as the miRNA*. The miRNA and miRNA* may be derived from opposing arms of the pri-miRNA and pre-miRNA. miRNA* sequences may be found in libraries of cloned miRNAs but typically at lower frequency than the miRNAs.

Although initially present as a double-stranded species with miRNA*, the miRNA eventually becomes incorporated as a single-stranded RNA into a ribonucleoprotein complex known as the RNA-induced silencing complex (RISC). Various proteins can form the RISC, which can lead to variability in specificity for miRNA/miRNA* duplexes, binding site of the target gene, activity of miRNA (repress or activate), and which strand of the miRNA/miRNA* duplex is loaded in to the RISC.

When the miRNA strand of the miRNA:miRNA* duplex is loaded into the RISC, the miRNA* is removed and degraded. The strand of the miRNA:miRNA* duplex that is loaded into the RISC is the strand whose 5′ end is less tightly paired. In cases where both ends of the miRNA:miRNA* have roughly equivalent 5′ pairing, both miRNA and miRNA* may have gene silencing activity.

The RISC identifies target nucleic acids based on high levels of complementarity between the miRNA and the mRNA, especially by nucleotides 2-7 of the miRNA.

A number of studies have looked at the base-pairing requirement between miRNA and its mRNA target for achieving efficient inhibition of translation (reviewed by Bartel 2004, Cell 116-281). In mammalian cells, the first 8 nucleotides of the miRNA may be important (Doench & Sharp 2004 GenesDev 2004-504). However, other parts of the microRNA may also participate in mRNA binding. Moreover, sufficient base pairing at the 3′ can compensate for insufficient pairing at the 5′ (Brennecke et al, 2005 PLoS 3-e85). Computation studies, analyzing miRNA binding on whole genomes have suggested a specific role for bases 2-7 at the 5′ of the miRNA in target binding but the role of the first nucleotide, found usually to be “A” was also recognized (Lewis et at 2005 Cell 120-15). Similarly, nucleotides 1-7 or 2-8 were used to identify and validate targets by Krek et al. (2005, Nat Genet 37-495).

The target sites in the mRNA may be in the 5′ UTR, the 3′ UTR or in the coding region. Interestingly, multiple miRNAs may regulate the same mRNA target by recognizing the same or multiple sites. The presence of multiple miRNA binding sites in most genetically identified targets may indicate that the cooperative action of multiple RISCs provides the most efficient translational inhibition.

miRNAs may direct the RISC to downregulate gene expression by either of two mechanisms: mRNA cleavage or translational repression. The miRNA may specify cleavage of the mRNA if the mRNA has a certain degree of complementarity to the miRNA. When a miRNA guides cleavage, the cut is typically between the nucleotides pairing to residues 10 and 11 of the miRNA. Alternatively, the miRNA may repress translation if the miRNA does not have the requisite degree of complementarity to the miRNA. Translational repression may be more prevalent in animals since animals may have a lower degree of complementarity between the miRNA and binding site.

It should be noted that there may be variability in the 5′ and 3′ ends of any pair of miRNA and miRNA*. This variability may be due to variability in the enzymatic processing of Drosha and Dicer with respect to the site of cleavage. Variability at the 5′ and 3′ ends of miRNA and miRNA* may also be due to mismatches in the stem structures of the pri-miRNA and pre-miRNA. The mismatches of the stem strands may lead to a population of different hairpin structures. Variability in the stem structures may also lead to variability in the products of cleavage by Drosha and Dicer.

The term “microRNA mimic” or “miRNA mimic” refers to synthetic non-coding RNAs that are capable of entering the RNAi pathway and regulating gene expression. miRNA mimics imitate the function of endogenous miRNAs and can be designed as mature, double stranded molecules or mimic precursors (e.g., or pre-miRNAs). miRNA mimics can be comprised of modified or unmodified RNA, DNA, RNA-DNA hybrids, or alternative nucleic acid chemistries (e.g., LNAs or 2′-O,4′-C-ethylene-bridged nucleic acids (ENA)). For mature, double stranded miRNA mimics, the length of the duplex region can vary between 13-33, 18-24 or 21-23 nucleotides. The miRNA may also comprise a total of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides. The sequence of the miRNA may be the first 13-33 nucleotides of the pre-miRNA. The sequence of the miRNA may also be the last 13-33 nucleotides of the pre-miRNA.

Preparation of miRNAs mimics can be effected by any method known in the art such as chemical synthesis or recombinant methods.

It will be appreciated from the description provided herein above that contacting cells with a miRNA may be effected by transfecting the cells with e.g. the mature double stranded miRNA, the pre-miRNA or the pri-miRNA.

The pre-miRNA sequence may comprise from 45-90, 60-80 or 60-70 nucleotides.

The pri-miRNA sequence may comprise from 45-30,000, 50-25,000, 100-20,000, 1,000-1,500 or 80-100 nucleotides.

Antisense—Antisense is a single stranded RNA designed to prevent or inhibit expression of a gene by specifically hybridizing to its mRNA. Downregulation of a MLKL, RIPK3 or RIPK1 can be effected using an antisense polynucleotide capable of specifically hybridizing with an mRNA transcript encoding MLKL, RIPK3 or RIPK1, respectively.

Design of antisense molecules which can be used to efficiently downregulate a MLKL, RIPK3 or RIPK1 must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof.

The prior art teaches of a number of delivery strategies which can be used to efficiently deliver oligonucleotides into a wide variety of cell types [see, for example, Jääskeläinen et al. Cell Mol Biol Lett. (2002) 7(2):236-7; Gait, Cell Mol Life Sci. (2003) 60(5):844-53; Martino et al. J Biomed Biotechnol. (2009) 2009:410260; Grijalvo et al. Expert Opin Ther Pat. (2014) 24(7):801-19; Falzarano et al, Nucleic Acid Ther. (2014) 24(1):87-100; Shilakari et al. Biomed Res Int. (2014) 2014: 526391; Prakash et al. Nucleic Acids Res. (2014) 42(13):8796-807 and Asseline et al. J Gene Med. (2014) 16(7-8):157-65].

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. Biotechnol Bioeng 65: 1-9 (1999)]. Such algorithms have been successfully used to implement an antisense approach in cells.

In addition, several approaches for designing and predicting efficiency of specific oligonucleotides using an in vitro system were also published (Matveeva et al., Nature Biotechnology 16: 1374-1375 (1998)].

Thus, the generation of highly accurate antisense design algorithms and a wide variety of oligonucleotide delivery systems, enable an ordinarily skilled artisan to design and implement antisense approaches suitable for downregulating expression of known sequences without having to resort to undue trial and error experimentation.

Nucleic acid agents can also operate at the DNA level as summarized infra.

Downregulation of MLKL, RIPK3 or RIPK1 can also be achieved by inactivating the gene (e.g., MLKL, RIPK3 or RIPK1) via introducing targeted mutations involving loss-of function alterations (e.g. point mutations, deletions and insertions) in the gene structure.

As used herein, the phrase “loss-of-function alterations” refers to any mutation in the DNA sequence of a gene (e.g., MLKL, RIPK3 or RIPK1) which results in downregulation of the expression level and/or activity of the expressed product, i.e., the mRNA transcript and/or the translated protein. Non-limiting examples of such loss-of-function alterations include a missense mutation, i.e., a mutation which changes an amino acid residue in the protein with another amino acid residue and thereby abolishes the enzymatic activity of the protein; a nonsense mutation, i.e., a mutation which introduces a stop codon in a protein, e.g., an early stop codon which results in a shorter protein devoid of the enzymatic activity; a frame-shift mutation, i.e., a mutation, usually, deletion or insertion of nucleic acid(s) which changes the reading frame of the protein, and may result in an early termination by introducing a stop codon into a reading frame (e.g., a truncated protein, devoid of the enzymatic activity), or in a longer amino acid sequence (e.g., a readthrough protein) which affects the secondary or tertiary structure of the protein and results in a non-functional protein, devoid of the enzymatic activity of the non-mutated polypeptide; a readthrough mutation due to a frame-shift mutation or a modified stop codon mutation (i.e., when the stop codon is mutated into an amino acid codon), with an abolished enzymatic activity; a promoter mutation, i.e., a mutation in a promoter sequence, usually 5′ to the transcription start site of a gene, which results in down-regulation of a specific gene product; a regulatory mutation, i.e., a mutation in a region upstream or downstream, or within a gene, which affects the expression of the gene product; a deletion mutation, i.e., a mutation which deletes coding nucleic acids in a gene sequence and which may result in a frame-shift mutation or an in-frame mutation (within the coding sequence, deletion of one or more amino acid codons); an insertion mutation, i.e., a mutation which inserts coding or non-coding nucleic acids into a gene sequence, and which may result in a frame-shift mutation or an in-frame insertion of one or more amino acid codons; an inversion, i.e., a mutation which results in an inverted coding or non-coding sequence; a splice mutation i.e., a mutation which results in abnormal splicing or poor splicing; and a duplication mutation, i.e., a mutation which results in a duplicated coding or non-coding sequence, which can be in-frame or can cause a frame-shift.

According to specific embodiments loss-of-function alteration of a gene may comprise at least one allele of the gene.

The term “allele” as used herein, refers to any of one or more alternative forms of a gene locus, all of which alleles relate to a trait or characteristic. In a diploid cell or organism, the two alleles of a given gene occupy corresponding loci on a pair of homologous chromosomes.

According to other specific embodiments loss-of-function alteration of a gene comprises both alleles of the gene. In such instances the e.g. MLKL, RIPK3 or RIPK1 may be in a homozygous form or in a heterozygous form. According to this embodiment, homozygosity is a condition where both alleles at the e.g. MLKL, RIPK3 or RIPK1 locus are characterized by the same nucleotide sequence. Heterozygosity refers to different conditions of the gene at the e.g. MLKL, RIPK3 or RIPK1 locus.

Methods of introducing nucleic acid alterations to a gene of interest are well known in the art [see for example Menke D. Genesis (2013) 51: -618; Capecchi, Science (1989) 244:1288-1292; Santiago et al. Proc Natl Acad Sci USA (2008) 105:5809-5814; International Patent Application Nos. WO 2014085593, WO 2009071334 and WO 2011146121; U.S. Pat. Nos. 8,771,945, 8,586,526, 6,774,279 and UP Patent Application Publication Nos. 20030232410, 20050026157, US20060014264; the contents of which are incorporated by reference in their entireties] and include targeted homologous recombination, site specific recombinases, PB transposases and genome editing by engineered nucleases. Agents for introducing nucleic acid alterations to a gene of interest can be designed publically available sources or obtained commercially from Transposagen, Addgene and Sangamo Biosciences.

Following is a description of various exemplary methods used to introduce nucleic acid alterations to a gene of interest and agents for implementing same that can be used according to specific embodiments of the present invention.

Genome Editing using engineered endonucleases—this approach refers to a reverse genetics method using artificially engineered nucleases to cut and create specific double-stranded breaks at a desired location(s) in the genome, which are then repaired by cellular endogenous processes such as, homology directed repair (HDR) and non-homologous end-joining (NFfEJ). NFfEJ directly joins the DNA ends in a double-stranded break, while HDR utilizes a homologous sequence as a template for regenerating the missing DNA sequence at the break point. In order to introduce specific nucleotide modifications to the genomic DNA, a DNA repair template containing the desired sequence must be present during HDR. Genome editing cannot be performed using traditional restriction endonucleases since most restriction enzymes recognize a few base pairs on the DNA as their target and the probability is very high that the recognized base pair combination will be found in many locations across the genome resulting in multiple cuts not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs) and CRISPR/Cas system.

Meganucleases—Meganucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp) thus making them naturally very specific for cutting at a desired location. This can be exploited to make site-specific double-stranded breaks in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence. Alternatively, DNA interacting amino acids of the meganuclease can be altered to design sequence specific meganucleases (see e.g., U.S. Pat. No. 8,021,867). Meganucleases can be designed using the methods described in e.g., Certo, M T et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8,148,098; or 8,163,514, the contents of each are incorporated herein by reference in their entirety. Alternatively, meganucleases with site specific cutting characteristics can be obtained using commercially available technologies e.g., Precision Biosciences' Directed Nuclease Editor™ genome editing technology.

ZFNs and TALENs—Two distinct classes of engineered nucleases, zinc-finger nucleases (ZFNs) and transcription activator-like effector nucleases (TALENs), have both proven to be effective at producing targeted double-stranded breaks (Christian et al., 2010; Kim et al., 1996; Li et al., 2011; Mahfouz et al., 2011; Miller et al., 2010).

Basically, ZFNs and TALENs restriction endonuclease technology utilizes a non-specific DNA cutting enzyme which is linked to a specific DNA binding domain (either a series of zinc finger domains or TALE repeats, respectively). Typically a restriction enzyme whose DNA recognition site and cleaving site are separate from each other is selected. The cleaving portion is separated and then linked to a DNA binding domain, thereby yielding an endonuclease with very high specificity for a desired sequence. An exemplary restriction enzyme with such properties is Fokl. Additionally Fokl has the advantage of requiring dimerization to have nuclease activity and this means the specificity increases dramatically as each nuclease partner recognizes a unique DNA sequence. To enhance this effect, Fokl nucleases have been engineered that can only function as heterodimers and have increased catalytic activity. The heterodimer functioning nucleases avoid the possibility of unwanted homodimer activity and thus increase specificity of the double-stranded break.

Thus, for example to target a specific site, ZFNs and TALENs are constructed as nuclease pairs, with each member of the pair designed to bind adjacent sequences at the targeted site. Upon transient expression in cells, the nucleases bind to their target sites and the Fokl domains heterodimerize to create a double-stranded break. Repair of these double-stranded breaks through the nonhomologous end-joining (NHEJ) pathway most often results in small deletions or small sequence insertions. Since each repair made by NHEJ is unique, the use of a single nuclease pair can produce an allelic series with a range of different deletions at the target site. The deletions typically range anywhere from a few base pairs to a few hundred base pairs in length, but larger deletions have successfully been generated in cell culture by using two pairs of nucleases simultaneously (Carlson et al., 2012; Lee et al., 2010). In addition, when a fragment of DNA with homology to the targeted region is introduced in conjunction with the nuclease pair, the double-stranded break can be repaired via homology directed repair to generate specific modifications (Li et al., 2011; Miller et al., 2010; Urnov et al., 2005).

Although the nuclease portions of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers typically found in repeats that are 3 bp apart and are found in diverse combinations in a variety of nucleic acid interacting proteins. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from e.g., Sangamo Biosciences™ (Richmond, Calif.).

Method for designing and obtaining TALENs are described in e.g. Reyon et al. Nature Biotechnology 2012 May; 30(5):460-5; Miller et al. Nat Biotechnol. (2011) 29: 143-148; Cermak et al. Nucleic Acids Research (2011) 39 (12): e82 and Zhang et al. Nature Biotechnology (2011) 29 (2): 149-53. A recently developed web-based program named Mojo Hand was introduced by Mayo Clinic for designing TAL and TALEN constructs for genome editing applications (can be accessed through www.talendesign.org). TALEN can also be designed and obtained commercially from e.g., Sangamo Biosciences™ (Richmond, Calif.).

CRISPR-Cas system—Many bacteria and archaea contain endogenous RNA-based adaptive immune systems that can degrade nucleic acids of invading phages and plasmids. These systems consist of clustered regularly interspaced short palindromic repeat (CRISPR) genes that produce RNA components and CRISPR associated (Cas) genes that encode protein components. The CRISPR RNAs (crRNAs) contain short stretches of homology to specific viruses and plasmids and act as guides to direct Cas nucleases to degrade the complementary nucleic acids of the corresponding pathogen. Studies of the type II CRISPR/Cas system of Streptococcus pyogenes have shown that three components form an RNA/protein complex and together are sufficient for sequence-specific nuclease activity: the Cas9 nuclease, a crRNA containing 20 base pairs of homology to the target sequence, and a trans-activating crRNA (tracrRNA) (Jinek et al. Science (2012) 337: 816-821.). It was further demonstrated that a synthetic chimeric guide RNA (gRNA) composed of a fusion between crRNA and tracrRNA could direct Cas9 to cleave DNA targets that are complementary to the crRNA in vitro. It was also demonstrated that transient expression of Cas9 in conjunction with synthetic gRNAs can be used to produce targeted double-stranded brakes in a variety of different species (Cho et al., 2013; Cong et al., 2013; DiCarlo et al., 2013; Hwang et al., 2013a,b; Jinek et al., 2013; Mali et al., 2013).

The CRIPSR/Cas system for genome editing contains two distinct components: a gRNA and an endonuclease e.g. Cas9.

The gRNA is typically a 20 nucleotide sequence encoding a combination of the target homologous sequence (crRNA) and the endogenous bacterial RNA that links the crRNA to the Cas9 nuclease (tracrRNA) in a single chimeric transcript. The gRNA/Cas9 complex is recruited to the target sequence by the base-pairing between the gRNA sequence and the complement genomic DNA. For successful binding of Cas9, the genomic target sequence must also contain the correct Protospacer Adjacent Motif (PAM) sequence immediately following the target sequence. The binding of the gRNA/Cas9 complex localizes the Cas9 to the genomic target sequence so that the Cas9 can cut both strands of the DNA causing a double-strand break. Just as with ZFNs and TALENs, the double-stranded brakes produced by CRISPR/Cas can undergo homologous recombination or NHEJ.

The Cas9 nuclease has two functional domains: RuvC and HNH, each cutting a different DNA strand. When both of these domains are active, the Cas9 causes double strand breaks in the genomic DNA.

A significant advantage of CRISPR/Cas is that the high efficiency of this system coupled with the ability to easily create synthetic gRNAs enables multiple genes to be targeted simultaneously. In addition, the majority of cells carrying the mutation present biallelic mutations in the targeted genes.

However, apparent flexibility in the base-pairing interactions between the gRNA sequence and the genomic DNA target sequence allows imperfect matches to the target sequence to be cut by Cas9.

Modified versions of the Cas9 enzyme containing a single inactive catalytic domain, either RuvC- or HNH-, are called ‘nickases’. With only one active nuclease domain, the Cas9 nickase cuts only one strand of the target DNA, creating a single-strand break or ‘nick’. A single-strand break, or nick, is normally quickly repaired through the HDR pathway, using the intact complementary DNA strand as the template. However, two proximal, opposite strand nicks introduced by a Cas9 nickase are treated as a double-strand break, in what is often referred to as a ‘double nick’ CRISPR system. A double-nick can be repaired by either NHEJ or HDR depending on the desired effect on the gene target. Thus, if specificity and reduced off-target effects are crucial, using the Cas9 nickase to create a double-nick by designing two gRNAs with target sequences in close proximity and on opposite strands of the genomic DNA would decrease off-target effect as either gRNA alone will result in nicks that will not change the genomic DNA.

Modified versions of the Cas9 enzyme containing two inactive catalytic domains (dead Cas9, or dCas9) have no nuclease activity while still able to bind to DNA based on gRNA specificity. The dCas9 can be utilized as a platform for DNA transcriptional regulators to activate or repress gene expression by fusing the inactive enzyme to known regulatory domains. For example, the binding of dCas9 alone to a target sequence in genomic DNA can interfere with gene transcription.

There are a number of publically available tools available to help choose and/or design target sequences as well as lists of bioinformatically determined unique gRNAs for different genes in different species such as the Feng Zhang lab's Target Finder, the Michael Boutros lab's Target Finder (E-CRISP), the RGEN Tools: Cas-OFFinder, the CasFinder: Flexible algorithm for identifying specific Cas9 targets in genomes and the CRISPR Optimal Target Finder.

In order to use the CRISPR system, both gRNA and Cas9 should be expressed in a target cell. The insertion vector can contain both cassettes on a single plasmid or the cassettes are expressed from two separate plasmids. CRISPR plasmids are commercially available such as the px330 plasmid from Addgene.

“Hit and run” or “in-out”—involves a two-step recombination procedure. In the first step, an insertion-type vector containing a dual positive/negative selectable marker cassette is used to introduce the desired sequence alteration. The insertion vector contains a single continuous region of homology to the targeted locus and is modified to carry the mutation of interest. This targeting construct is linearized with a restriction enzyme at a one site within the region of homology, electroporated into the cells, and positive selection is performed to isolate homologous recombinants. These homologous recombinants contain a local duplication that is separated by intervening vector sequence, including the selection cassette. In the second step, targeted clones are subjected to negative selection to identify cells that have lost the selection cassette via intrachromosomal recombination between the duplicated sequences. The local recombination event removes the duplication and, depending on the site of recombination, the allele either retains the introduced mutation or reverts to wild type. The end result is the introduction of the desired modification without the retention of any exogenous sequences.

The “double-replacement” or “tag and exchange” strategy—involves a two-step selection procedure similar to the hit and run approach, but requires the use of two different targeting constructs. In the first step, a standard targeting vector with 3′ and 5′ homology arms is used to insert a dual positive/negative selectable cassette near the location where the mutation is to be introduced. After electroporation and positive selection, homologously targeted clones are identified. Next, a second targeting vector that contains a region of homology with the desired mutation is electroporated into targeted clones, and negative selection is applied to remove the selection cassette and introduce the mutation. The final allele contains the desired mutation while eliminating unwanted exogenous sequences.

Site-Specific Recombinases—The Cre recombinase derived from the P1 bacteriophage and Flp recombinase derived from the yeast Saccharomyces cerevisiae are site-specific DNA recombinases each recognizing a unique 34 base pair DNA sequence (termed “Lox” and “FRT”, respectively) and sequences that are flanked with either Lox sites or FRT sites can be readily removed via site-specific recombination upon expression of Cre or Flp recombinase, respectively. For example, the Lox sequence is composed of an asymmetric eight base pair spacer region flanked by 13 base pair inverted repeats. Cre recombines the 34 base pair lox DNA sequence by binding to the 13 base pair inverted repeats and catalyzing strand cleavage and religation within the spacer region. The staggered DNA cuts made by Cre in the spacer region are separated by 6 base pairs to give an overlap region that acts as a homology sensor to ensure that only recombination sites having the same overlap region recombine.

Basically, the site specific recombinase system offers means for the removal of selection cassettes after homologous recombination. This system also allows for the generation of conditional altered alleles that can be inactivated or activated in a temporal or tissue-specific manner. Of note, the Cre and Flp recombinases leave behind a Lox or FRT “scar” of 34 base pairs. The Lox or FRT sites that remain are typically left behind in an intron or 3′ UTR of the modified locus, and current evidence suggests that these sites usually do not interfere significantly with gene function.

Thus, Cre/Lox and Flp/FRT recombination involves introduction of a targeting vector with 3′ and 5′ homology arms containing the mutation of interest, two Lox or FRT sequences and typically a selectable cassette placed between the two Lox or FRT sequences. Positive selection is applied and homologous recombinants that contain targeted mutation are identified. Transient expression of Cre or Flp in conjunction with negative selection results in the excision of the selection cassette and selects for cells where the cassette has been lost. The final targeted allele contains the Lox or FRT scar of exogenous sequences.

Transposases—As used herein, the term “transposase” refers to an enzyme that binds to the ends of a transposon and catalyzes the movement of the transposon to another part of the genome.

As used herein the term “transposon” refers to a mobile genetic element comprising a nucleotide sequence which can move around to different positions within the genome of a single cell. In the process the transposon can cause mutations and/or change the amount of a DNA in the genome of the cell.

A number of transposon systems that are able to also transpose in cells e.g. vertebrates have been isolated or designed, such as Sleeping Beauty [Izsvák and Ivics Molecular Therapy (2004) 9, 147-156], piggyBac [Wilson et al. Molecular Therapy (2007) 15, 139-145], To12 [Kawakami et al. PNAS (2000) 97 (21): 11403-11408] or Frog Prince [Miskey et al. Nucleic Acids Res. Dec 1, (2003) 31(23): 6873-6881]. Generally, DNA transposons translocate from one DNA site to another in a simple, cut-and-paste manner. Each of these elements has their own advantages, for example, Sleeping Beauty is particularly useful in region-specific mutagenesis, whereas Tol2 has the highest tendency to integrate into expressed genes. Hyperactive systems are available for Sleeping Beauty and piggyBac. Most importantly, these transposons have distinct target site preferences, and can therefore introduce sequence alterations in overlapping, but distinct sets of genes. Therefore, to achieve the best possible coverage of genes, the use of more than one element is particularly preferred. The basic mechanism is shared between the different transposases, therefore piggyBac (PB) is described as an example.

PB is a 2.5 kb insect transposon originally isolated from the cabbage looper moth, Trichoplusia ni. The PB transposon consists of asymmetric terminal repeat sequences that flank a transposase, PBase. PBase recognizes the terminal repeats and induces transposition via a “cut-and-paste” based mechanism, and preferentially transposes into the host genome at the tetranucleotide sequence TTAA. Upon insertion, the TTAA target site is duplicated such that the PB transposon is flanked by this tetranucleotide sequence. When mobilized, PB typically excises itself precisely to reestablish a single TTAA site, thereby restoring the host sequence to its pretransposon state. After excision, PB can transpose into a new location or be permanently lost from the genome.

Typically, the transposase system offers an alternative means for the removal of selection cassettes after homologous recombination quit similar to the use Cre/Lox or Flp/FRT. Thus, for example, the PB transposase system involves introduction of a targeting vector with 3′ and 5′ homology arms containing the mutation of interest, two PB terminal repeat sequences at the site of an endogenous TTAA sequence and a selection cassette placed between PB terminal repeat sequences. Positive selection is applied and homologous recombinants that contain targeted mutation are identified. Transient expression of PBase removes in conjunction with negative selection results in the excision of the selection cassette and selects for cells where the cassette has been lost. The final targeted allele contains the introduced mutation with no exogenous sequences.

For PB to be useful for the introduction of sequence alterations, there must be a native TTAA site in relatively close proximity to the location where a particular mutation is to be inserted.

Genome editing using recombinant adeno-associated virus (rAAV) platform—this genome-editing platform is based on rAAV vectors which enable insertion, deletion or substitution of DNA sequences in the genomes of live mammalian cells. The rAAV genome is a single-stranded deoxyribonucleic acid (ssDNA) molecule, either positive- or negative-sensed, which is about 4.7 kb long. These single-stranded DNA viral vectors have high transduction rates and have a unique property of stimulating endogenous homologous recombination in the absence of double-strand DNA breaks in the genome. One of skill in the art can design a rAAV vector to target a desired genomic locus and perform both gross and/or subtle endogenous gene alterations in a cell. rAAV genome editing has the advantage in that it targets a single allele and does not result in any off-target genomic alterations. rAAV genome editing technology is commercially available, for example, the rAAV GENESIS™ system from Horizon™ (Cambridge, UK).

It will be appreciated that the agent can be a mutagen that causes random mutations and the cells exhibiting downregulation of the expression level and/or activity of MLKL, RIPK3 or RIPK1 may be selected.

The mutagens may be, but are not limited to, genetic, chemical or radiation agents. For example, the mutagen may be ionizing radiation, such as, but not limited to, ultraviolet light, gamma rays or alpha particles. Other mutagens may include, but not be limited to, base analogs, which can cause copying errors; deaminating agents, such as nitrous acid; intercalating agents, such as ethidium bromide; alkylating agents, such as bromouracil; transposons; natural and synthetic alkaloids; bromine and derivatives thereof; sodium azide; psoralen (for example, combined with ultraviolet radiation). The mutagen may be a chemical mutagen such as, but not limited to, ICR191, 1,2,7,8-diepoxy-octane (DEO), 5-azaC, N-methyl-N-nitrosoguanidine (MNNG) or ethyl methane sulfonate (EMS).

Methods for qualifying efficacy and detecting sequence alteration are well known in the art and include, but not limited to, DNA sequencing, electrophoresis, an enzyme-based mismatch detection assay and a hybridization assay such as PCR, RT-PCR, RNase protection, in-situ hybridization, primer extension, Southern blot, Northern Blot and dot blot analysis.

Sequence alterations in a specific gene can also be determined at the protein level using e.g. chromatography, electrophoretic methods, immunodetection assays such as ELISA and western blot analysis and immunohistochemistry.

In addition, one ordinarily skilled in the art can readily design a knock-in/knock-out construct including positive and/or negative selection markers for efficiently selecting transformed cells that underwent a homologous recombination event with the construct. Positive selection provides a means to enrich the population of clones that have taken up foreign DNA. Non-limiting examples of such positive markers include glutamine synthetase, dihydrofolate reductase (DHFR), markers that confer antibiotic resistance, such as neomycin, hygromycin, puromycin, and blasticidin S resistance cassettes. Negative selection markers are necessary to select against random integrations and/or elimination of a marker sequence (e.g. positive marker). Non-limiting examples of such negative markers include the herpes simplex-thymidine kinase (HSV-TK) which converts ganciclovir (GCV) into a cytotoxic nucleoside analog, hypoxanthine phosphoribosyltransferase (HPRT) and adenine phosphoribosytransferase (ARPT).

Ribozymes

Another agent capable of downregulating a MLKL, RIPK3 or RIPK1 is a ribozyme molecule capable of specifically cleaving an mRNA transcript encoding a MLKL, RIPK3 or RIPK1, respectively. Ribozymes are being increasingly used for the sequence-specific inhibition of gene expression by the cleavage of mRNAs encoding proteins of interest [Welch et al., Curr Opin Biotechnol. 9:486-96 (1998)]. The possibility of designing ribozymes to cleave any specific target RNA has rendered them valuable tools in both basic research and therapeutic applications. In the therapeutics area, ribozymes have been exploited to target viral RNAs in infectious diseases, dominant oncogenes in cancers and specific somatic mutations in genetic disorders [Welch et al., Clin Diagn Virol. 10:163-71 (1998)]. Most notably, several ribozyme gene therapy protocols for HIV patients are already in Phase 1 trials. More recently, ribozymes have been used for transgenic animal research, gene target validation and pathway elucidation. Several ribozymes are in various stages of clinical trials. ANGIOZYME was the first chemically synthesized ribozyme to be studied in human clinical trials. ANGIOZYME specifically inhibits formation of the VEGF-r (Vascular Endothelial Growth Factor receptor), a key component in the angiogenesis pathway. Ribozyme Pharmaceuticals, Inc., as well as other firms have demonstrated the importance of anti-angiogenesis therapeutics in animal models. HEPTAZYME, a ribozyme designed to selectively destroy Hepatitis C Virus (HCV) RNA, was found effective in decreasing Hepatitis C viral RNA in cell culture assays (Ribozyme Pharmaceuticals, Incorporated—WEB home page).

DNAzymes

Another agent capable of downregulating a MLKL, RIPK3 or RIPK1 is a DNAzyme molecule capable of specifically cleaving an mRNA transcript or DNA sequence of the MLKL, RIPK3 or RIPK1, respectively. DNAzymes are single-stranded polynucleotides which are capable of cleaving both single and double stranded target sequences (Breaker, R. R. and Joyce, G. Chemistry and Biology 1995;2:655; Santoro, S. W. & Joyce, G. F. Proc. Natl, Acad. Sci. USA 1997; 943:4262) A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, L M [Curr Opin Mol Ther 4:119-21 (2002)].

Examples of construction and amplification of synthetic, engineered DNAzymes recognizing single and double-stranded target cleavage sites have been disclosed in U.S. Pat. No. 6,326,174 to Joyce et al. DNAzymes of similar design directed against the human Urokinase receptor were recently observed to inhibit Urokinase receptor expression, and successfully inhibit colon cancer cell metastasis in vivo (Itoh et al, 20002, Abstract 409, Ann Meeting Am Soc Gen Ther www.asgt.org). In another application, DNAzymes complementary to bcr-ab1 oncogenes were successful in inhibiting the oncogenes expression in leukemia cells, and lessening relapse rates in autologous bone marrow transplant in cases of CML and ALL.

An additional method of regulating the expression of an MLKL, RIPK3 or RIPK1 gene in cells is via triplex forming oligonuclotides (TFOs). Recent studies have shown that TFOs can be designed which can recognize and bind to polypurine/polypirimidine regions in double-stranded helical DNA in a sequence-specific manner. These recognition rules are outlined by Maher III, L. J., et al., Science, 1989; 245:725-730; Moser, H. E., et al., Science, 1987; 238:645-630; Beal, P. A., et al, Science, 1992; 251:1360-1363; Cooney, M., et al., Science, 1988; 241:456-459; and Hogan, M. E., et al., EP Publication 375408. Modification of the oligonuclotides, such as the introduction of intercalators and backbone substitutions, and optimization of binding conditions (pH and cation concentration) have aided in overcoming inherent obstacles to TFO activity such as charge repulsion and instability, and it was recently shown that synthetic oligonucleotides can be targeted to specific sequences (for a recent review see Seidman and Glazer, J Clin Invest 2003; 112:487-94).

In general, the triplex-forming oligonucleotide has the sequence correspondence:

| | | | | |
|---|---|---|---|---|
| oligo | 3′--A | G | G | T |
| duplex | 5′--A | G | C | T |
| duplex | 3′--T | C | G | A |

However, it has been shown that the A-AT and G-GC triplets have the greatest triple helical stability (Reither and Jeltsch, BMC Biochem, 2002, Sep. 12, Epub). The same authors have demonstrated that TFOs designed according to the A-AT and G-GC rule do not form non-specific triplexes, indicating that the triplex formation is indeed sequence specific.

Thus for any given sequence in the MLKL, RIPK3 or RIPK1 regulatory region a triplex forming sequence may be devised. Triplex-forming oligonucleotides preferably are at least 15, more preferably 25, still more preferably 30 or more nucleotides in length, up to 50 or 100 bp.

Transfection of cells (for example, via cationic liposomes) with TFOs, and formation of the triple helical structure with the target DNA induces steric and functional changes, blocking transcription initiation and elongation, allowing the introduction of desired sequence changes in the endogenous DNA and resulting in the specific downregulation of gene expression. Examples of such suppression of gene expression in cells treated with TFOs include knockout of episomal supFG1 and endogenous HPRT genes in mammalian cells (Vasquez et al., Nucl Acids Res. 1999; 27:1176-81, and Puri, et al, J Biol Chem, 2001; 276:28991-98), and the sequence- and target specific downregulation of expression of the Ets2 transcription factor, important in prostate cancer etiology (Carbone, et al, Nucl Acid Res. 2003; 31:833-43), and the pro-inflammatory ICAM-1 gene (Besch et al, J Biol Chem, 2002; 277:32473-79). In addition, Vuyisich and Beal have recently shown that sequence specific TFOs can bind to dsRNA, inhibiting activity of dsRNA-dependent enzymes such as RNA-dependent kinases (Vuyisich and Beal, Nuc. Acids Res 2000; 28:2369-74).

Additionally, TFOs designed according to the abovementioned principles can induce directed mutagenesis capable of effecting DNA repair, thus providing both downregulation and upregulation of expression of endogenous genes (Seidman and Glazer, J Clin Invest 2003; 112:487-94). Detailed description of the design, synthesis and administration of effective TFOs can be found in U.S. Patent Application Nos. 2003 017068 and 2003 0096980 to Froehler et al, and 2002 0128218 and 2002 0123476 to Emanuele et al, and U.S. Pat. No. 5,721,138 to Lawn.

Down-Regulation at the Polypeptide Level

One example, of an agent capable of downregulating a MLKL, RIPK3 or RIPK1 is an antibody or antibody fragment capable of specifically binding MLKL, RIPK3 or RIPK1, respectively. Preferably, the antibody specifically binds at least one epitope of a MLKL, RIPK3 or RIPK1. As used herein, the term “epitope” refers to any antigenic determinant on an antigen to which the paratope of an antibody binds. Epitopic determinants usually consist of chemically active surface groupings of molecules such as amino acids or carbohydrate side chains and usually have specific three dimensional structural characteristics, as well as specific charge characteristics.

As MLKL, RIPK3 or RIPK1 are typically localized intracellularly, an antibody or antibody fragment capable of specifically binding MLKL, RIPK3 or RIPK1 is typically an intracellular antibody.

It will be appreciated that targeting of a particular compartment within the cell can be achieved using intracellular antibodies (also known as “intrabodies”). These are essentially single chain antibodies to which intracellular localization signals have been added (e.g., ER, mitochondrial, nuclear, cytoplasmic). This technology has been successfully applied in the art (for review, see Richardson and Marasco, 1995, TIBTECH vol. 13: 306-310). Intrabodies have been shown to virtually eliminate the expression of otherwise abundant cell surface receptors and to inhibit a protein function within a cell (See, for example, Richardson et al., 1995, Proc. Natl. Acad. Sci. USA 92: 3137-3141; Deshane et al., 1994, Gene Ther. 1: 332-337; Marasco et al., 1998 Human Gene Ther 9: 1627-42; Shaheen et al., 1996 J. Virol. 70: 3392-400; Werge, T. M. et al., 1990, FEBS Letters 274:193-198; Carlson, J. R. 1993 Proc. Natl. Acad. Sci. USA 90:7427-7428; Biocca, S. et al., 1994, Bio/Technology 12: 396-399; Chen, S-Y. et al., 1994, Human Gene Therapy 5:595-601; Duan, L et al., 1994, Proc. Natl. Acad. Sci. USA 91:5075-5079; Chen, S-Y. et al., 1994, Proc. Natl. Acad. Sci. USA 91:5932-5936; Beerli, R. R. et al., 1994, J. Biol. Chem. 269:23931-23936; Mhashilkar, A. M. et al., 1995, EMBO J. 14:1542-1551; PCT Publication No. WO 94/02610 by Marasco et al.; and PCT Publication No. WO 95/03832 by Duan et al.).

To prepare an intracellular antibody expression vector, the cDNA encoding the antibody light and heavy chains specific for the target protein of interest are isolated, typically from a hybridoma that secretes a monoclonal antibody specific for the marker. Hybridomas secreting anti-marker monoclonal antibodies, or recombinant monoclonal antibodies, can be prepared using methods known in the art. Once a monoclonal antibody specific for the marker protein is identified (e.g., either a hybridoma-derived monoclonal antibody or a recombinant antibody from a combinatorial library), DNAs encoding the light and heavy chains of the monoclonal antibody are isolated by standard molecular biology techniques. For hybridoma derived antibodies, light and heavy chain cDNAs can be obtained, for example, by PCR amplification or cDNA library screening. For recombinant antibodies, such as from a phage display library, cDNA encoding the light and heavy chains can be recovered from the display package (e.g., phage) isolated during the library screening process and the nucleotide sequences of antibody light and heavy chain genes are determined. For example, many such sequences are disclosed in Kabat, E. A., et al. (1991) Sequences of Proteins of Immunological Interest, Fifth Edition, U.S. Department of Health and Human Services, NIH Publication No. 91-3242 and in the “Vbase” human germline sequence database. Once obtained, the antibody light and heavy chain sequences are cloned into a recombinant expression vector using standard methods.

For cytoplasmic expression of the light and heavy chains, the nucleotide sequences encoding the hydrophobic leaders of the light and heavy chains are removed. An intracellular antibody expression vector can encode an intracellular antibody in one of several different forms. For example, in one embodiment, the vector encodes full-length antibody light and heavy chains such that a full-length antibody is expressed intracellularly. In another embodiment, the vector encodes a full-length light chain but only the VH/CH1 region of the heavy chain such that a Fab fragment is expressed intracellularly. In another embodiment, the vector encodes a single chain antibody (scFv) wherein the variable regions of the light and heavy chains are linked by a flexible peptide linker [e.g., (Gly₄Ser)₃ and expressed as a single chain molecule. To inhibit marker activity in a cell, the expression vector encoding the intracellular antibody is introduced into the cell by standard transfection methods, as discussed hereinbefore.

Once antibodies are obtained, they may be tested for activity, for example via ELISA.

Another agent which can be used along with some embodiments of the invention to downregulate MLKL, RIPK3 or RIPK1 is an aptamer. As used herein, the term “aptamer” refers to double stranded or single stranded RNA molecule that binds to specific molecular target, such as a protein. Various methods are known in the art which can be used to design protein specific aptamers. The skilled artisan can employ SELEX (Systematic Evolution of Ligands by Exponential Enrichment) for efficient selection as described in Stoltenburg R, Reinemann C, and Strehlitz B (Biomolecular engineering (2007) 24(4):381-403).

Another agent capable of downregulating MLKL, RIPK3 or RIPK1 would be any molecule which binds to and/or cleaves MLKL, RIPK3 or RIPK1. Such molecules can be a small molecule, MLKL, RIPK3 or RIPK1 antagonists, or MLKL, RIPK3 or RIPK1 inhibitory peptide.

It will be appreciated that a non-functional analogue of at least a catalytic or binding portion of MLKL, RIPK3 or RIPK1 can be also used as an agent which downregulates MLKL, RIPK3 or RIPK1.

Alternatively or additionally, small molecule or peptides can be used which interfere with MLKL, RIPK3 or RIPK1 protein function (e.g., catalytic or interaction).

Another agent which can be used along with some embodiments of the invention to downregulate MLKL, RIPK3 or RIPK1 is a molecule which prevents MLKL, RIPK3 or RIPK1 activation or substrate binding.

Exemplary agents for downregulating an activity or expression of MLKL, RIPK3 or RIPK1 are described in detail hereinabove.

According to one embodiment, the agent for downregulating an activity or expression of RIPK1 comprises Necrostatin-1 (as described in detail above).

According to one embodiment, the agent for downregulating an activity or expression of RIPK3 comprises GSK' 872 (as described in detail above).

According to one embodiment, the agent for downregulating an activity or expression of MLKL comprises necrosulfonamide (as described in detail above).

According to one embodiment, the agent is capable of downregulating an endocytic activity of the MLKL without compromising a necroptotic activity of the MLKL. Screening for agents capable of downregulating an endocytic activity of the MLKL without compromising a necroptotic activity of the MLKL can be carried out using any method known in the art and as described herein. Thus, for example, a cell can be contacted with an agent and tested for endocytic activity and for necroptosis. For example, endocytic activity can be tested by measuring the rate of intracellular degradation of a ligand (e.g. TNF) after binding to its receptor (e.g. TNF receptor) using a ligand-receptor uptake assay, e.g. as discussed in detail the materials and methods section below. Furthermore, testing for necroptosis of a cell can be carried out, for example, by testing the release of cytoplasmic component such as the enzyme lactic dehydrogenase, or by staining for dead cells such as by staining for Annexin-V and 7-amino actinomycin D (or propidium iodine), or for DCFH-DA and propidium iodide (as described above), or using the Cytotoxicity Detection Kit (such as the one available from Roche Applied Science).

According to one embodiment, endocytosis of a cell surface receptor is reduced by about 5%, 10%, 20%, 30%, 40%, 50%, 60%, 70%, 80%, 90%, 95%, 99% or by 100% as compared to a cell not administered with the agent of some embodiments of the invention.

According to another embodiment, the cell is further contacted with a ligand.

According to another embodiment, the method is effected ex vivo.

According to one embodiment, the method is effected in vivo.

According to one embodiment, the MLKL downregulating agent and the ligand are used concomitantly.

According to another embodiment, the MLKL downregulating agent and the ligand are used sequentially, wherein the ligand is used, for example, 30 minutes, 1 hour, 6 hours, 12 hours, 24 hours, 48 hours, 72 hours, 96 hours, a week, a month or more after the MLKL downregulating agent. Such a determination is well within the capacity of one of skill in the art.

According to an alternative or an additional aspect, there is provided a method of treating a disease or disorder in which modulating endocytosis of a cell surface receptor capable of ligand induced endocytosis is beneficial, the method comprising administering to a subject an agent capable of downregulating an endocytic activity of MLKL without compromising necroptotic activity of the MLKL, thereby treating the disease or disorder in the subject.

According to one embodiment, the method further comprises administering to the subject the ligand.

According to one embodiment, the MLKL downregulating agent and the ligand are administered concomitantly.

According to another embodiment, the MLKL downregulating agent and the ligand are administered sequentially, wherein the ligand is used, for example, 30 minutes, 1 hour, 6 hours, 12 hours, 24 hours, 48 hours, 72 hours, 96 hours, a week, a month or more after the MLKL downregulating agent. Such a determination is well within the capacity of one of skill in the art.

According to one embodiment, the disease or disorder comprises, but is not limited to, a cancer, an immunodeficiency, an inflammatory disease (e.g. an inflammatory bowel disease), a neurodegeneration (e.g. Alzheimer's disease, Parkinson's disease, diffuse Lewy body disease (LBD)), a Chronic Obstructive Pulmonary Disease (COPD), a thrombocytopenia, a chronic infection (e.g. Chronic hepatitis B infection), an autoimmune disease and a diabetes.

According to one embodiment, the subject is immunocompromised. According to another embodiment, the subject has a low cellular sensitivity to a ligand.

According to another embodiment, the subject may benefit from increased cellular sensitivity to a ligand.

According to one embodiment, when the subject has diabetes, the ligand may comprise insulin.

According to one embodiment, when the subject has neurodegeneration, the ligand may comprise a neurotransmitter.

According to another embodiment, when the subject has cancer, the ligand may comprise an anti-tumor ligand such as a TNF family member (as discussed in detail above), or any other ligand which is capable of potentiating an antitumor immune responses.

According to an alternative or an additional aspect, there is provided a method of enhancing immunotherapy in a subject in need thereof, the method comprising modulating endocytosis of a cell surface receptor capable of ligand induced endocytosis according to the method of some embodiments of the invention, wherein the ligand is capable of modulating T cell activation and enhancing an immune response.

The term “immunotherapy” as used herein refers to an array of treatment strategies based upon the concept of modulating the immune system to achieve a prophylactic and/or therapeutic goal.

The term “modulating T cell activation” as used herein refers to reducing or enhancing an activity of a T lymphocyte, including but not limited to, effector T cells, cytotoxic T cells, helper T cells, regulatory T cells (e.g. suppressor T cells), naïve T cells and memory T cells.

T cell activation can be measured by any method known in the art, including but not limited to the following: detection and/or quantitation of cell surface markers such as CD69, CD25, HLA-DR, CD62L, CD154 and/or the production of IL-2, calcium mobilization, ZAP-70 phosphorylation, LAT phosphorylation, Lck phosphorylation; NF-κB activation, MEK activation, NFAT activation, Ap-1 activation; T cell proliferation and cytotoxicity (i.e. defined as the ability to kill target cells).

According to one embodiment, modulating T cell activation is used to enhance an activity of a T cell (e.g. cytotoxic T cell, helper T cells).

According to one embodiment, modulating T cell activation is used to decrease T cell anergy.

According to another embodiment, modulating T cell activation is used to target and destroy diseased cells (e.g. cancer cells).

Enhancing T cell activation may be beneficial in a subject having an infection or at risk of having an infection, a subject having a suppressed immune system or suppressed immune response or at risk of having a suppressed immune system or suppressed immune response, as known in the art. Examples of infections that cause immunosuppression include, but are not limited to, human immunodeficiency virus (HIV) infection, cytomegalovirus infection, vaccinia virus infection, and F. tularenesis bacterial infection. Conditions under which immune suppression occurs include, but are not limited to, severe immunodeficiencies (e.g. SCID), advanced age, chemotherapy, radiation therapy, irradiation and upon severe burn.

According to one embodiment, enhancing T cell activation is beneficial in a subject having a diseases cell such as a cell associated with a hyperproliferative disease e.g. cancer (e.g. solid tumor, tumor metastasis, hematological malignancy) or a cell associated with an inflammatory disease, as discussed in further detail hereinbelow.

According to one embodiment, the immunotherapy comprises a ligand which activates a T cell, including but not limited to, peptides, DNA and glycoproteins. Exemplary ligands capable of modulating T cell activation and enhancing an immune response include, but are not limited to, GM-CSF, IL-2, IL-12, IFN-γ, IL-4, TNF family members (e.g. TNF-α), IL-1, hematopoietic factor flt3L, CD40L, B7.1 co-stimulatory molecules and B7.2 co-stimulatory molecules, and toll-like receptor agonists.

According to one embodiment, the immune response is enhanced by about 5%, 10%, 20%, 30%, 40%, 50%, 60%, 70%, 80%, 90%, 95%, 99% or by 100% as compared to a subject not treated by an agent capable of modulating endocytosis of a cell surface receptor.

According to one embodiment, the effect of the immunotherapy is enhanced by about 5%, 10%, 20%, 30%, 40%, 50%, 60%, 70%, 80%, 90%, 95%, 99% or by 100% as compared to a subject not treated by an agent capable of modulating endocytosis of a cell surface receptor.

According to a specific embodiment, when the disease is a cancer, the ligand comprises a TNF family member [For additional details see e.g. Schaer D. J Immunother Cancer. (2014) 2: 7, incorporated herein by reference].

Under physiological conditions exosomes can play a role in cell to cell interactions. Accordingly, the present invention is further directed to the use of exosomes as delivery vehicles of genetic material to a target cell.

According to one aspect of the invention, there is provided a method of inducing necroptosis or inflammation in a subject in need thereof, the method comprising administering to the subject a therapeutically effective amount of a population of exosomes comprising a component of a necroptosis activation pathway.

As used herein, the phrase “inducing necroptosis or inflammation” refers to triggering a necroptosis or inflammation as a way of treating a disease.

Typically, by causing local inflammation at a site of disease, various immune cells are attracted to the site of inflammation and act to eliminate the diseased cells. Furthermore, induction of necroptosis leads to death of the diseased cell.

Inducing necroptosis or inflammation may be beneficial in a subject having an inflammatory disease, a cancer or a hyperproliferative disorder (i.e. a condition in which non-cancerous (i.e. non-neoplastic) cells overproduce in response to a particular growth factor).

Examples of inflammatory diseases are provided hereinabove.

Examples of hyperproliferative disorders include, but are not limited to, diabetic retinopathy, psoriasis, endometriosis, macular degenerative disorders and benign growth disorders such as prostate enlargement and lipomas.

Examples of cancer include, but are not limited to, carcinoma, lymphoma, blastoma, sarcoma, and leukemia. Particular examples of cancerous diseases but are not limited to: Myeloid leukemia such as Chronic myelogenous leukemia. Acute myelogenous leukemia with maturation. Acute promyelocytic leukemia, Acute nonlymphocytic leukemia with increased basophils, Acute monocytic leukemia. Acute myelomonocytic leukemia with eosinophilia; Malignant lymphoma, such as Birkitt's Non-Hodgkin's; Lymphoctyic leukemia, such as Acute lumphoblastic leukemia. Chronic lymphocytic leukemia; Myeloproliferative diseases, such as Solid tumors Benign Meningioma, Mixed tumors of salivary gland, Colonic adenomas; Adenocarcinomas, such as Small cell lung cancer, Kidney, Uterus, Prostate, Bladder, Ovary, Colon, Sarcomas, Liposarcoma, myxoid, Synovial sarcoma, Rhabdomyosarcoma (alveolar), Extraskeletel myxoid chonodrosarcoma, Ewing's tumor; other include Testicular and ovarian dysgerminoma, Retinoblastoma, Wilms' tumor, Neuroblastoma, Malignant melanoma, Mesothelioma, breast, skin, prostate, and ovarian.

The method of some embodiments of the invention is affected by administering to the subject a therapeutically effective amount of a population of exosomes comprising a component of a necroptosis activation pathway.

According to one embodiment, the component of the necroptosis activation pathway comprises a MLKL.

According to one embodiment, the MLKL comprises a phosphorylated MLKL.

According to one embodiment, the MLKL comprises a constitutively active mutant (e.g. constitutively phosphorylated).

According to one embodiment, the phosphorylated MLKL comprises a phospho-mimetic mutation at an amino acid residue that is the target of phosphorylation by RIPK3.

According to a specific embodiment, the phospho-mimetic mutation comprises a threonine to glutamic acid modification in amino acid 357 and/or a serine to aspartic acid modification in amino acid 358 of the MLKL.

According to one embodiment, the phosphorylated MLKL comprises a phospho-mimetic mutation at an amino acid residue within the ATP-binding pocket of the MLKL.

According to a specific embodiment, the phospho-mimetic mutation comprises a lysine to methionine modification in amino acid 230 and/or a glutamine to alanine modification in amino acid 356 of the MLKL.

According to one embodiment, the component of the necroptosis activation pathway comprises a receptor interacting protein kinase 1 (RIPK1) or a receptor interacting protein kinase 3 (RIPK3).

According to one embodiment, the RIPK1 or RIPK3 comprises a phosphorylated RIPK1 or RIPK3.

According to one embodiment, the RIPK1 or RIPK3 comprises a constitutively active mutant (e.g. constitutively phosphorylated).

Exosomes comprising a component of a necroptosis activation pathway (e.g. MLKL, RIPK3 or RIPK1) may be obtained using any method known in the art. For example, exosomes may be genetically engineered to express the component of the necroptosis activation pathway (e.g. MLKL, RIPK3 or RIPK1).

Accordingly, the exogenous genetic material (i.e. a component of the necroptosis activation pathway, e.g. MLKL, RIPK3 or RIPK1) can be introduced into the exosomes by a various techniques. For example, the exosomes may be loaded by electroporation or the use of a transfection reagent. Despite the small size of exosomes (e.g. typically between 20-200 nm), previous publications have illustrated that it is possible to use electroporation and transfection reagent to load the exosomes with the exogenous genetic material including DNA and RNA (see for example European Patent No. EP2419144). Typical voltages are in the range of 20 V/cm to 1000 V/cm, such as 20 V/cm to 100 V/cm with capacitance typically between 25 μF and 250 μF, such as between 25 μF and 125 μF. Alternatively, conventional transfection reagent can be used for transfection of exosomes with genetic material, such as but not limited to, cationic liposomes.

As exosomes are derived from a variety of different cells, cells (e.g. antigen presenting cells such as dendritic cells and macrophages) may be genetically engineered with an exogenous genetic material (including DNA and RNA) for expression of a component of the necroptosis activation pathway (e.g. MLKL, RIPK3 or RIPK1). These cells are then cultured for an ample amount of time to produce exosomes (e.g. for 1, 2, 3, 4, 5, 6, 12, 24, 48, 72, 96 hours, for several days e.g. 1, 2, 3, 4, 5, 6, 7, 8, 9, 10, 12, 14, 21 or 30 days, or for several weeks e.g. 1, 2, 3, 4, 5, 6, 7, 8, 10, 12 or 14 weeks) prior to harvesting of the exosomes.

According to some embodiments of the invention, the exosomes are targeted to a desired cell or tissue (e.g. diseased cells including cancerous cells, inflammatory cells or hyperproliferative cells). This targeting is achieved by expressing on the surface of the exosomes a binding agent which binds to a cell surface moiety expressed on the surface of the cell to be targeted. For example, the exosomes can be targeted to particular cell types or tissues by expressing on their surface a binding agent such as a protein, a peptide or a glycolipid molecule. For example, suitable peptides are those which bind to cell surface moieties such as receptors or their ligands found on the cell surface of the cell to be targeted. Examples of suitable binding agents are short peptides, scFv and complete proteins, so long as the binding agent can be expressed on the surface of the exosome and does not interfere with expression of the component of the necroptosis activation pathway.

Determination that the exosomes comprise a component of the necroptosis activation pathway (e.g. MLKL, RIPK3 or RIPK1) can be carried out using any of the methods described herein, e.g. by Western blot, ELISA, FACS, MACS. Likewise, determination that the exosomes comprise a binding agent can be carried out using any method known in the art.

Each of the downregulating agents, ligands or population of exosomes described hereinabove can be administered to the individual per se or as part of a pharmaceutical composition which also includes a physiologically acceptable carrier. The purpose of a pharmaceutical composition is to facilitate administration of the active ingredient to an organism.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the downregulating agents, ligands or population of exosomes accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, inrtaperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

The term “tissue” refers to part of an organism consisting of cells designed to perform a function or functions. Examples include, but are not limited to, brain tissue, retina, skin tissue, hepatic tissue, pancreatic tissue, bone, cartilage, connective tissue, blood tissue, muscle tissue, cardiac tissue brain tissue, vascular tissue, renal tissue, pulmonary tissue, gonadal tissue, hematopoietic tissue.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (e.g. downregulating agents, ligands or population of exosomes) effective to prevent, alleviate or ameliorate symptoms of a disorder (e.g., a disease associated with activation of a necroptosis activation pathway, necroptosis or inflammation) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Animal models for necroptosis are described, for example, in Pasparakis M. and Vandenabeele P., Nature 517, 311-320 (2015); Christofferson D. E. et al., Annu. Rev. Physiol. 76, 129-150 (2014); and Zhao H. et al., Cell Death and Disease. (2015) 6, e1975; doi:10.1038/cddis.2015.316. Animal models for inflammation are described for example in Webb D R, Biochem Pharmacol. (2014) 87(1):121-30.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide the active ingredient at a sufficient amount to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

According to one embodiment, there is provided a pharmaceutical composition comprising as an active ingredient an agent capable of downregulating an endocytic activity of MLKL without compromising necroptotic activity of the MLKL, and a pharmaceutically accepted carrier.

According to one embodiment, the pharmaceutical composition further comprises a ligand capable of binding to a cell surface receptor capable of ligand induced endocytosis.

According to one embodiment, there is provided a pharmaceutical composition comprising as an active ingredient a population of exosomes comprising a component of a necroptosis activation pathway and a pharmaceutically accepted carrier.

The composition can be substantially enriched for exosomes. For example, the composition can be substantially free of cells, cellular debris, or non-exosomal proteins, peptides, or nucleic acids (such as biological molecules not contained within the exosomes). Such a composition can be obtained by any method disclosed herein, such as through the use of one or more agents (e.g. antibody) for one or more exosomes.

According to one embodiment, the exosomes can comprise at least about 20%, 30%, 40%, 50%, 60%, 70%, 80%, 90%, 95% or 99% of the total composition, by weight or by mass.

According to one embodiment, the exosomes can comprise a heterogeneous or homogeneous population of exosomes. For example, a homogeneous population of exosomes comprises exosomes that are homogeneous as to one or more properties or characteristics (e.g. exosomes of a particular size, exosomes expressing a cell specific marker).

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

According to one embodiment, there is provided an article of manufacture comprising an agent capable of downregulating an endocytic activity of MLKL without compromising necroptotic activity of the MLKL, and a ligand capable of binding to a cell surface receptor capable of ligand induced endocytosis, being packaged in a packaging material and identified in print, in or on the packaging material for use in the treatment of a disease or disorder in which modulating endocytosis of a cell surface receptor capable of ligand induced endocytosis is beneficial.

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Experimental Procedures

Reagents

Bafilomycin A1 from Santa Cruz Biotechnology was applied to the cells at 100 nM. Chloroquine was applied at 25 μM, 4-hydroxytamoxifen was applied at 1 μM, and ionomycin was applied at 1 μM, ultra pure LPS, applied at 1 μg/ml (all from Sigma). Human ECF from Biological industries, mouse EGF from ProSpec and human TNF from Ybdy were biotinylated using an EZ-Link biotinylation kit (Thermo Fisher Scientific) and then applied to the cells at 5 μg/ml or injected intravenously into mice at a dose of 10 μg/mouse. Bovine serum albumin (BSA), tagged with gold particles (BSA-gold, CMC Utrecht), was applied to the cells at an optical density of 2. To trigger necroptosis, TNF (1000 units/ml) was applied together with the bivalentIAP (inhibitor of apoptosis protein) antagonist BV6 and the caspase inhibitor z-VAD-fmk, both from WuXu App Tec, at concentrations of 1 μM and 20 μM respectively. IL-1β ELISA kit was from eBiosicne.

Antibodies

The following antibodies were applied for Western blotting analysis:

Anti-human MLKL (GTX107538) from GeneTex; anti-mouse MLKL (Sab1302339), anti β-actin (A5441), anti-ERK (M5670) and anti-phospho-ERK (M8159) from Sigma; anti-human phospho MLKL (ab187091) and anti-mouse phospho MLKL (ab196436) from Abcam; anti-Flotillin-1 (610820), anti-TSG101 (612696), anti-phospho Tyrosine (61000), and anti-Rab 27a (558532) from BD Biosciences; anti-human RIPK3 (13526), anti-AKT (9272), anti-phospho AKT (4051) and anti-phospho-STAT3 (9131) from Cell Signaling; anti-HSP70 (EXOAB-hsp70A-1) and anti-CD9 (EXOAB-CD9A-1) from System Biosciences; anti-IL-1β (AF-401-NA) from R&D systems; anti-Alix (3A9) from BioLegend; anti-Hrs (A-5) and anti-EGFR (6F1) from Enzo Life Sciences; anti-Rab 27b (13412-1AP) from Proteintech; and anti-STAT3 (SC-8019) from Santa Cruz. Streptavidin-HRP (21124) was purchased from Thermo Fisher Scientific and HRP-conjugated antibodies from Jackson ImmunoResearch.

For immunofluorescence analysis the following antibodies were used:

EGFR conjugated to Alexa 647 (5588, Cell Signaling); antibodies against Rab7 (ab137029, Abcam) and EEA1 (610456, BD Bioscience); Cy2-conjugated goat anti-rabbit (111-225-144), Jackson ImmunoResearch; and Cy3-conjugated anti-mouse (AP124C, EMD Millipore).

For immune electron microscopy the following antibodies were used:

Anti-CD63 (MEM-259, DSHB) and anti-EGFR (20-E504, Fitzgerald). Secondary antibodies were 12 nm colloidal gold-conjugated donkey anti-sheep IgG (713-205-147), and nm colloidal gold-conjugated goat anti-mouse IgG (115-215-166, Jackson ImmunoResearch).

Cell Culture

Cells of the human HT-29 colorectal adenocarcinoma line were grown in McCoy's 5A medium. Cells of the HeLa cervical adenocarcinoma line and of the HepG2 hepatocellular carcinoma line, as well as mouse embryonic fibroblasts (MEFs) immortalized by expression of the SV40 large T antigen, were cultured in Dulbecco's modified Eagle's medium. Both media were supplemented with 10% fetal bovine serum (ITS), 100 U/ml penicillin, and 100 μg/ml streptomycin. Mouse bone marrow-derived macrophages (BMDM) and bone marrow-derived dendritic cells (RMDC) were produced as described previously [Kang T B et al. J Immunol. (2013) 173, 2976-2984].

RNA Interference

To knock down the expression of human Rab27a and Rab27b, RNAi duplex (Rab27a, 5′-UAUGUUUGUCCCAUUGGCAGCTT-3′ as set forth in SEQ ID NO: 1, Rab27b: 5′-UACUGUAGUGAUGAAUUUGGGTT-3′ as set forth in SEQ ID NO: 2) from Integrated DNA Technologies were used. Human MLKL expression was knocked down using 3′-UTR targeting lentiviral shRNA (Sigma).

inducible Expression of MLKL Mutants

The various mutants of MLKL were expressed inducibly in HT-29 cells in which the endogenous MLKL mRNA was constitutively knocked down. Expression was achieved by lentiviral infection with the GEV16/pF5x UAS system as described previously [Dunning C J et al. The EMBO Journal (2007) 26, 3227-3237, doi:10.1038/sj.emboj.7601748].

Mice

Mice carrying knocked-out MLKL allele were obtained from Taconic. Mice carrying knocked-out RIPK3 allele and mice carrying knocked-out RIPK1 allele were also used. All animal protocols were approved by the Institutional Animal Care and Use Committee (IACUC) of the Weizmann Institute of Science.

Collection and Quantification of Exosomes

In all experiments aimed at assessing the generation of exosomes by cultured cells, the FBS supplementing the growth medium was pre-depleted of bovine exosomes by centrifugation at 10,000×G for 18 hours. Except where otherwise indicated, the exosomes were collected after 12 hours of incubation. The stimuli whose effect on exosome generation was to be assessed were applied for the indicated times towards the end of this 12 hour period. Unless otherwise indicated, TBZ was applied for 4 hours. For recovery of exosomes from the mice, mouse blood samples were collected in MiniCollect®TUBEs (Greiner Bio-One) and centrifuged at 3000 rpm for 15 minutes. Mouse plasma and cell-growth media were passed through 0.2 μm filters and centrifuged at 10,000×G for 90 minutes, after which the pellet was suspended in phosphate-buffered saline (PBS) and re-sedimented by centrifugation as above. Size spectra and amounts of the particles in the exosome preparations were determined by Nanoparticle Tracking Analysis using the NanoSight NS300 device (Malvern Instruments) according to the manufacturer's instructions.

Ligand and Receptor Uptake Assays

Prior to ligand treatment the cells were incubated for 12 hours in serum-free medium. Biotinylated EGF and TNF were applied to the cells for 30 minutes on ice in CO₂-independent medium (Thermo Fisher Scientific), and this was followed by rinsing and rther incubation without those ligands at 37° C. For western blot analysis, cellular proteins were extracted at the indicated times in RIPA buffer (20 mM Tris-HCl pH 7.5, 150 mM NaCl, 1 mM EDTA, 1 mM EGTA, 1% NP-40, 0.5% sodium deoxycholate, 0.1% sodium dodecyl sulfate (SDS), protease and phosphatase inhibitors). For immunofluorescence microscopy, cells were fixed in 4% paraformaldehyde (PFA) in PBS and then stained with fluorescence-conjugated antibodies.

Fluorescence Microscopy

Immunostaining was performed as described previously [Yoon S. et al., Cell death and differentiation (2016) 23, 253-260]. Briefly, cells fixed with 4% PFA in PBS were permeabilized by incubation in methanol at −20° C. for 10 minutes, followed by incubation in blocking buffer (20% normal goat serum (NGS), 2% BSA, 0.3 M glycine and 0.1% Tween-20 in PBS, pH 7.2). Following incubation with the indicated antibodies, images were obtained using an Olympus IX 81 confocal microscope (Olympus Imaging), with a UPLSAPO 60×1.35 NA oil objective and FluoView FV1000 software (Olympus Imaging). Images were processed using Imaris software from Bitplane.

Transmission Electron Microscopy

HepG2 cells were incubated for 30 minutes at 37° C. in cell-growth medium containing BSA tagged with gold (BSA-gold) and 100 ng/ml EGF, and then fixed for 2 hours with Karnovsky's fixative (4% PFA, 2% glutaraldehyde, 5 mM CaCl₂ in 0.1 M cacodylate buffer, pH 7.4). The fixed cells were scraped, pelleted by centrifugation, embedded in 1.7% Nobel agar and postfixed with 1% osmium tetroxide, 0.5% potassium dichromate and 0.5% potassium hexacyanoferrate in 0.1 M cacodylate buffer. The pellet was stained and blocked with 2% aqueous uranyl acetate, then dehydrated with ethanol and embedded in graded Epon 812. Ultrathin sections (70-100 nm) were cut with Leica Ultracut UCT and analyzed using an FEI T12 Spirit electron microscope. Images were obtained with an Eagle CCD camera and processed by Image J software.

Immunogold Electron Microscopy

Cells were fixed for 2 hours at room temperature in freshly prepared 3% PFA, 0.1% glutaraldedyde in 0.1 M cacodylate buffer containing 5 mM CaCl₂. Pelleted fixed cells were infiltrated for 30 minutes in 10% gelatin at 37° C. Excess gelatin was removed by centrifugation at 37° C., followed by incubation at 4° C. for 24 hours. The fixed cell pellets were cryoprotected by overnight infiltration with 2.3 M sucrose in cacodylate buffer, then frozen by injection into liquid nitrogen. Ultrathin (75 nm) frozen sections were sliced at −110° C. on a Leica EM FC6 ultramicrotome and transferred to formvar-coated 200-mesh nickel grids. Sections were treated for 5 minutes with 3% NGS, 0.5% BSA, 0.1% glycine and 1% Tween-20 in PBS to block non-specific binding, and this was followed by incubation for 2 hours with the primary antibodies. After extensive rinsing with 0.1% glycine in PBS (PBS-glycine) the cells were further incubated for 30 minutes in colloidal gold-conjugated rabbit anti-mouse antibody. The grids were then washed in PBS-glycine and stained with 2% methyl cellulose/uranyl acetate.

Cell Death Tests

Cell death was quantified using the Cytotoxicity Detection Kit (Roche Applied Science) to determine the concentration of lactic dehydrogenase (LDH) in the mouse cell media or plasma.

Real-Time PCR Analysis

The mRNA of IL-1β was quantified, and the results of this assessment were presented, as described previously. [Kang T B et al., Immunity (2013) 38, 1-14]

Western Blot Analysis

Cells and liver samples were extracted in RIPA and in 1% SDS, respectively. Samples of the cell and liver lysates containing 20 μg protein (determined using the BCA protein assay kit; Thermo Fisher Scientific) and samples of exosomes derived from 2.3×10⁷ cells were loaded on SDS-polyacrylamide gel electrophoresis (PAGE) gels, electrophoresed, and transferred onto nitrocellulose membranes (Bio-Rad). The membranes were treated for 1 hour at room temperature with PBS containing 5% skimmed milk and 0.05% Tween-20 and then further incubated for 16 hours at 4° C. with either primary antibody in PBS containing 5% BSA or (for detection of biotinylated TNF or EGF) with streptavidin linked to horseradish peroxidase (Strep-HRP) in PBS containing 0.025% Tween-20 (PBS-Tween). After three washes in PBS-Tween the membranes were incubated for 1 hour at room temperature with the HRP-conjugated secondary antibody in PBS-Tween. After three more washes in PBS-Tween, blots were developed using the West Pico stable peroxidase solution (Thermo Fisher Scientific). Band intensity was quantified using the ImageJ software.

Statistics and Reproducibility

All of the presented data are representative results of at least two independent experiments. In all diagrams, values correspond to mean values of triplicate samples; error bars show standard deviations.

### Example 1

### Triggering the Necroptotic Signaling Pathway Enhances Exosome Generation

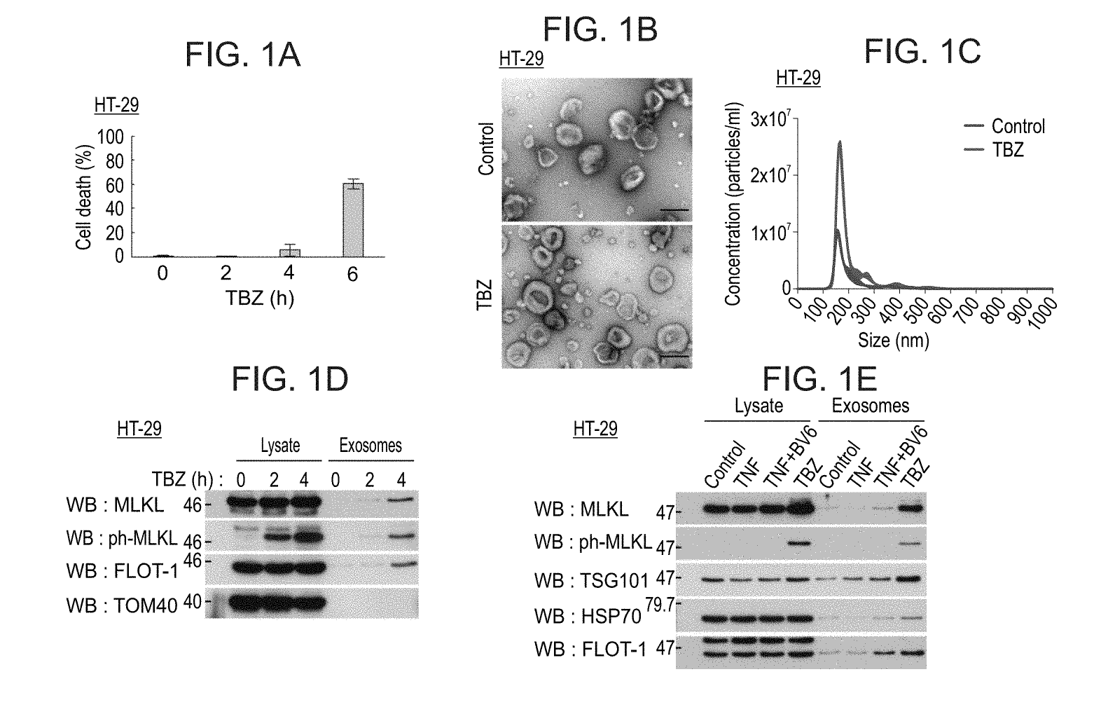

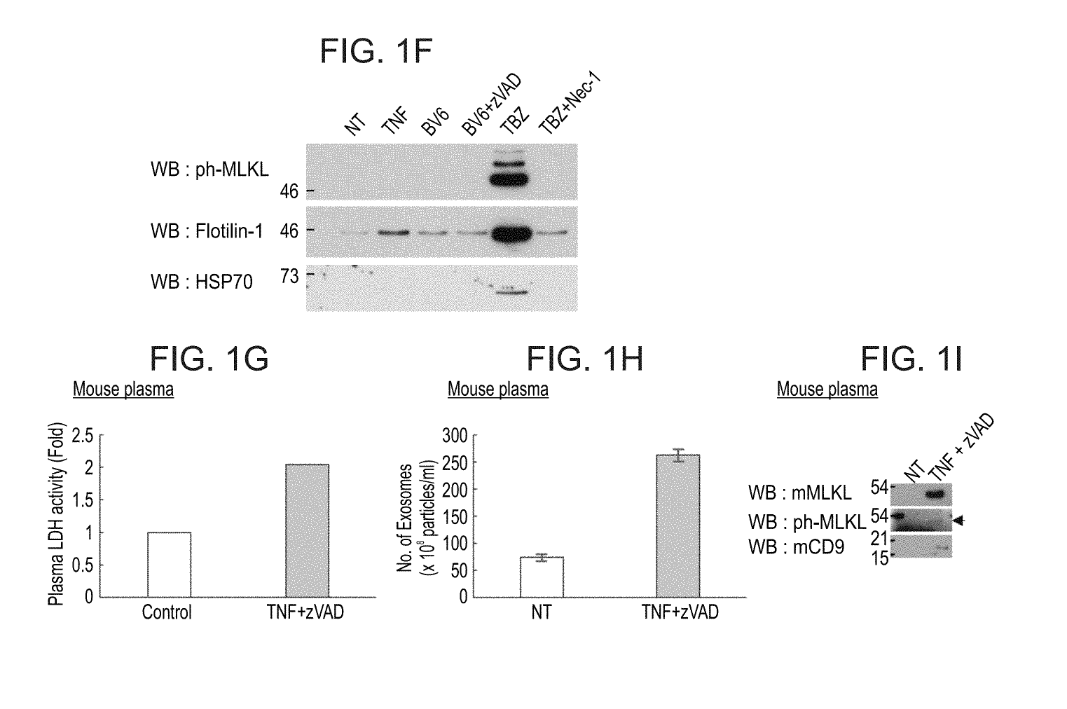

Since programmed necrotic death of cells is believed to serve for controlled release of pro-inflammatory intracellular components, the present inventors endeavored to thoroughly analyze the cellular constituents released after induction of necroptosis. Triggering necroptosis in HT29 cells by combined treatment with TNF, the SMAC mimetic agent BV6, and the caspase inhibitor zVAD (TBZ) it was surprisingly found that already at 4 hours of treatment, a time at which only very few of the cells have died (FIG. 1A), the content of particulate constituents of the cells in the media increased. In electron microscopic analysis of the material sedimented by ultra-centrifugation of the growth media of the cells it was found to contain doughnut shaped small membrane vesicles (FIG. 1B), the size of which could be defined by Nanoparticle Tracking Analysis to be at the range of 50-170 nm, which is characteristic of exosomes (FIG. 1C) [Colombo M et al., Annual review of cell and developmental biology (2014) 30: 255-289]. Western blot analysis confirmed presence of proteins characteristic of exosomes such as TSG101, HSP70, flotilin 1 and flotilin 2 and Alix, yet not of the mitochondrial protein TOM40, in the sedimented material, and of their increase in response to TBZ (FIG. 1D). Treating the cells with TNF and BV6 in the absence of zVAD resulted in milder increase in exosome generation, while treatment with TNF alone was without effect (FIG. 1E). The exosomes were found also to contain MLKL, a protein taking part in the signaling for necroptosis, at amounts that dramatically increased in response to TBZ. Like the MLKL molecules inside the TBZ treated cells, those extruded in exosome were found to be phophorylated at S358, a target for RIPK3-mediated phosphorylation in the necroptotic pathway (FIGS. 1D-E).

Triggering necroptosis by TNF depends on the protein kinase functions of RIPK1 and of RIPK3 and on phosphorylation of MLKL by the latter. In support of the involvement of the necroptotic signaling pathway in the enhancement of exosome generation by TBZ, such enhancement could not be observed in dermal fibroblasts derived from mice in which the RIPK1, RIPK3 or MLKL genes were knocked out (data not shown). Furthermore, necrostatin-1 (Nec-1), an inhibitor of the kinase function of RIPK1 [Degterev A et al., Nat Chem Biol (2008) 4: 313-321], was found to block this enhancement (FIG. 1F).

Injecting mice with TNF plus zVAD, which together trigger necroptosis in vivo [Duprez L. et al., Immunity (2011) 35: 908-918] (FIG. 1G) resulted in increased plasma level of exosomes (FIG. 1H) and in incorporation of phospho-MLKL into them (FIG. 1I).

### Example 2

### MLKL Deficiency Compromises Exosome Generation

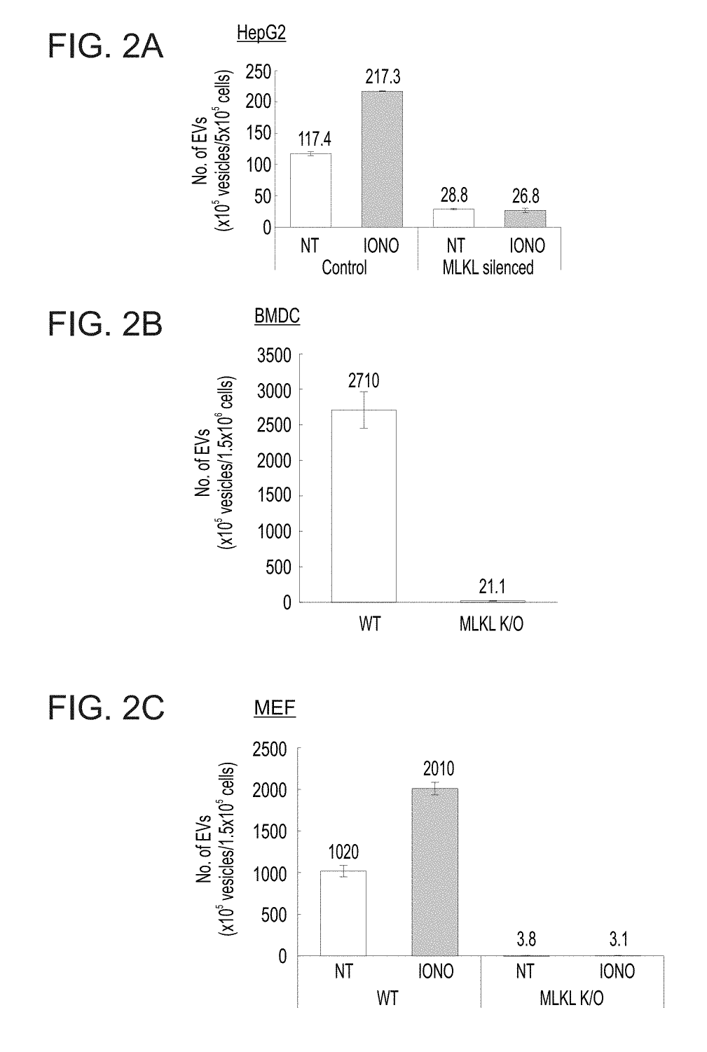

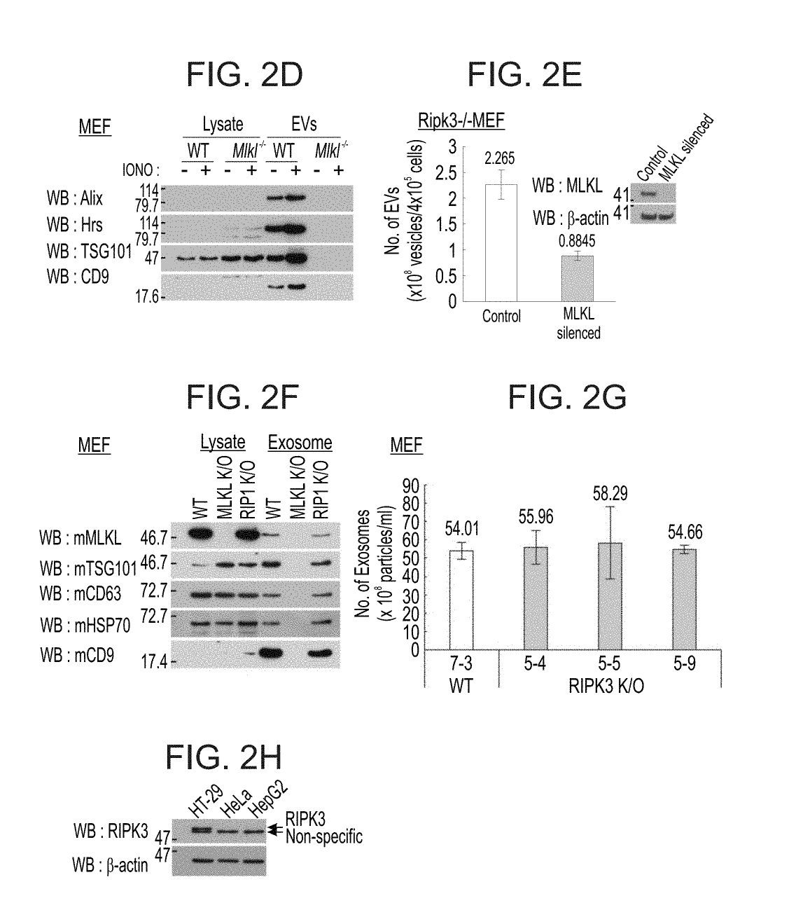

Surprisingly, it was found that both knockdown of MLKL and knockout of its gene, did not only compromise the enhancement of exosome generation by TBZ, but also resulted in a dramatic decrease in the amounts of exosomes generated by the cells in the absence of treatment by TBZ (FIGS. 2A-F). In contrast, deficiency of neither RIPK1 (FIG. 2F) or of RIPK3 (FIG. 2G), the two protein kinases acting upstream of MLKL in the necroptotic pathway, had any effect on the extent of constitutive exosome generation in the absence of TBZ. Normal extents of generation of exosomes were observed in cultures of MEFs derived from RIPK1 or RIPK3 knockout mice (FIGS. 2F-G) as well as in cultures of HeLa (not shown) and HepG2 cells (FIG. 2A) two cell lines that do not express the protein kinase RIPK3 (FIG. 2H). Just as in cells that do express RIPK3, knockdown of MLKL in the HeLa and HepG2 cells resulted in dramatic reduction in the extent of constitutive generation of exosomes by them (FIG. 2A and data not shown).

These findings suggested that MLKL contributes to the regulation of exosome generation and that it does so in two different manners: it mediates enhancement of exosome generation consequently to activation of the kinase function of RIPK3 and also contributes, independently of RIPK3 or of RIPK1, to the maintenance of constitutive generation of exosomes by cells.

### Example 3

### Assessment of the Structural Requirement for the Role of MLKL in Exosome Generation

To further explore the relationship between the role of MLKL in controlling exosome generation and its role in the mediation of necroptotic death, the impact of various mutations in MLKL on the two activities was compared.

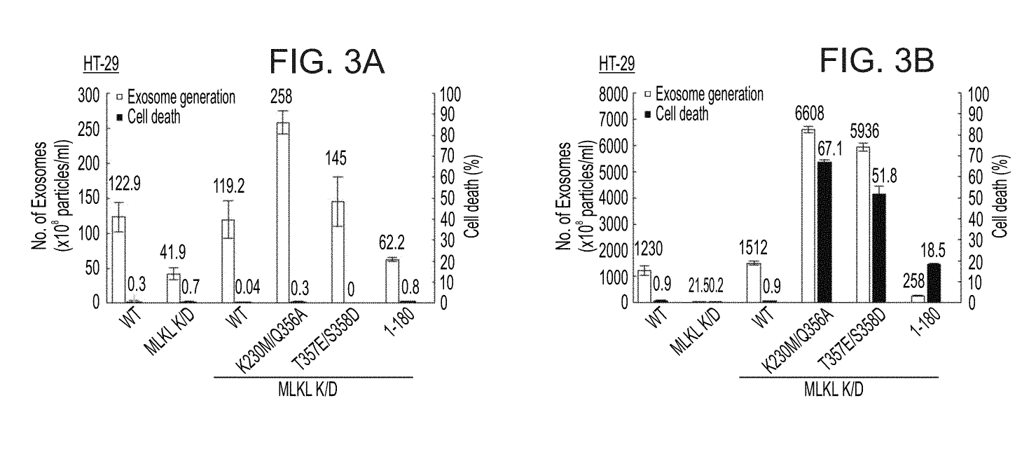

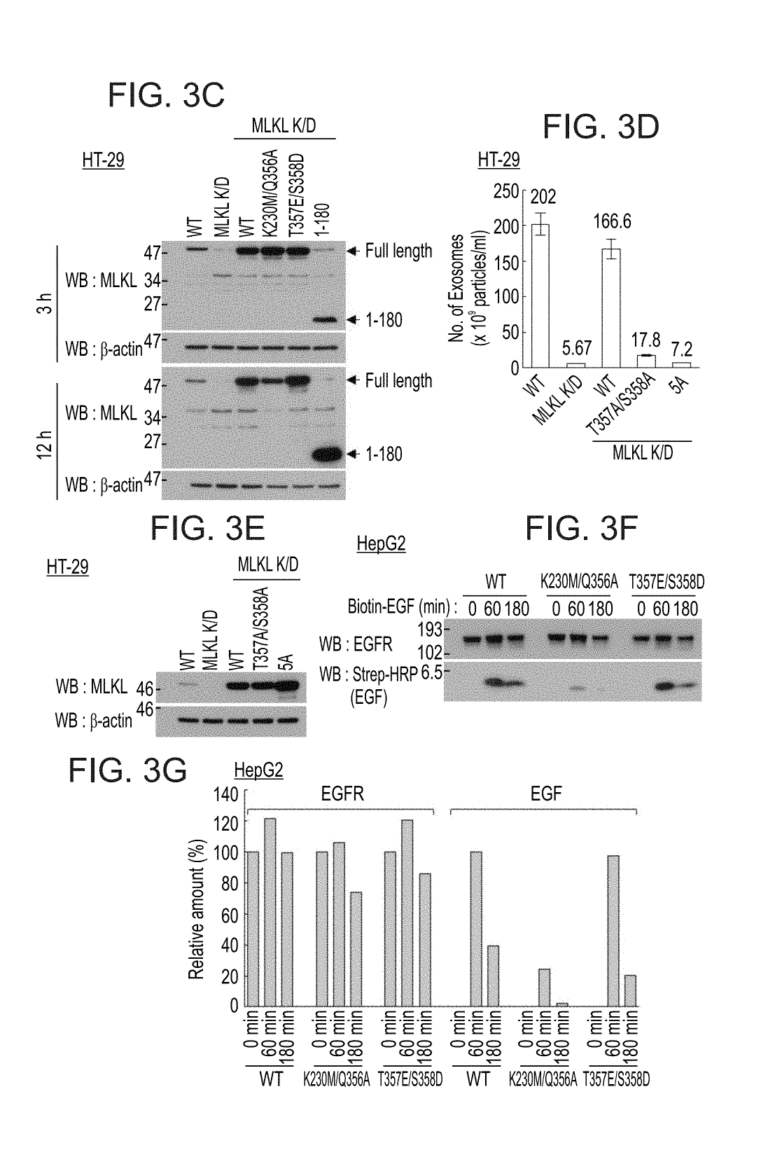

It has been previously shown that HT29 cells in which MLKL has been knocked down regain sensitivity to the induction of necroptosis by TBZ when expressing inducibly the cDNA for the wild-type protein [Yoon S. et al., Cell death and differentiation (2016) 23: 253-260]. The present inventors found them also to regain constitutive generation of exosomes (FIGS. 3A, 3B, 3D). Inducible expression of MLKL molecules with phospho-mimetic mutations of the residues that are the targets of phosphorylation by RIPK3 (T357E/S358D) and of MLKL molecules in which mutations within the ATP-binding pocket impose the conformational change that MLKL attains after phophorylation (K230M/Q356A) triggers death in the HT29 cells [Yoon et al. (2016), supra]. As shown in FIGS. 3A-C, it also triggered generation of exosomes by these cells at levels higher than in cells expressing the wild type protein. Moreover, the two mutants facilitated the intracellular degradation of EGF and EGFR following their uptake by cells (FIG. 3G), further demonstrating their ability to enhance endosomal trafficking. Conversely, replacing the RIPK3 targeted residues with alanine (T357A/S358A) compromised, not only the induction of necroptosis by TBZ as previously reported [Sun et al. (2012), supra], but also the generation of exosomes by the cells (FIG. 3D)—a rather unexpected finding in view of the finding that expression RIPK3 is not required for the contribution of MLKL to the maintenance of constitutive exosome generation. Cells inducibly expressing MLKL molecules in which—Lys5, Lys16 Arg17, and Lys50 Arg51—were replaced with alanine—a mutation that interferes with the binding of MLKL to lipids and was shown to arrest death induction by TBZ [Quarato G. et al., Molecular cell (2016) 61: 589-601], also failed to generate exosomes (‘5A’ in FIG. 3D).

The present inventors have found that induced expression of an MLKL deletion mutant corresponding to its 180 N-terminal amino acid coiled-coil region, which is believed to be constitutively folded at the conformation attained by the protein following its phosphorylation by MLKL, triggers necrotic death of the cells. Surprisingly, however, the amounts of exosomes generated by the cells expressing this mutant were significantly lower than those generated by cells expressing the wild-type protein (FIGS. 3A-B).

The findings above suggest that the structural requirement for the induction of cell death and for the facilitation of exosome generation by MLKL overlap, and yet are somewhat distinct. The conformational change that is attained by MLKL upon its phosphorylation, which is required for the mediation of necroptosis, also results in enhancement of exosome generation. However, unlike necroptosis, the latter function involves, not only the N-terminal coiled-coil portion of MLKL that is exposed upon MLKL phosphorylation, but also some structure(s) at its C-terminus. Furthermore, just like the necroptotic function MLKL, its contribution to exosome generation involves the residues within MLKL that RIPK3 phosphorylates, and yet unlike the induction of necroptosis, the contribution of MLKL to basal generation of exosomes is independent of RIPK3 itself.

### Example 4

### MLKL Controls the Accumulation of Intraluminal Vesicles in Multivesicular Bodies

Exosomes are derived from intracellular-structures called ‘multivesicular bodies’ (MVB). They accumulate within them as ‘intraluminal vesicles’ (ILV) and are released to the cells' exterior upon fusion of their membranes with the plasma membrane. This fusion can in some cells be enhanced by increased cytosolic calcium. Consistently, both in MEFs and in HepG2 cells, treatment with ionomycin, which initiates influx of calcium ions, yielded enhanced generation of exosomes. However, no such enhancement could be observed in MEFs or HepG2 cells deficient of MLKL (FIGS. 2A and 2C). This finding suggested that the low generation of exosomes by MLKL deficient cells does not reflect arrest of their release by cells but rather interference with the process of their actual generation within the cell.

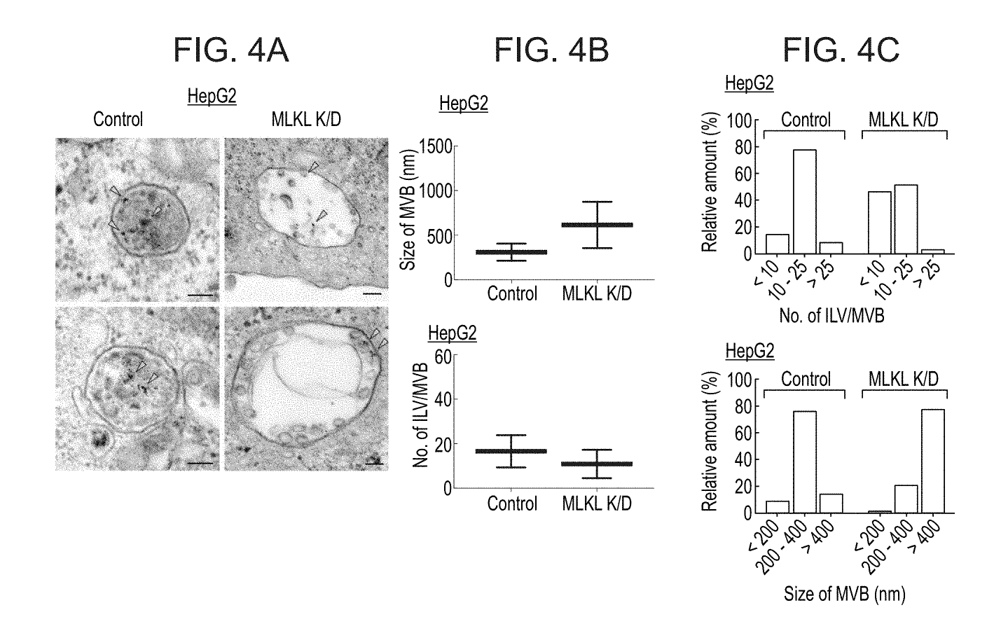

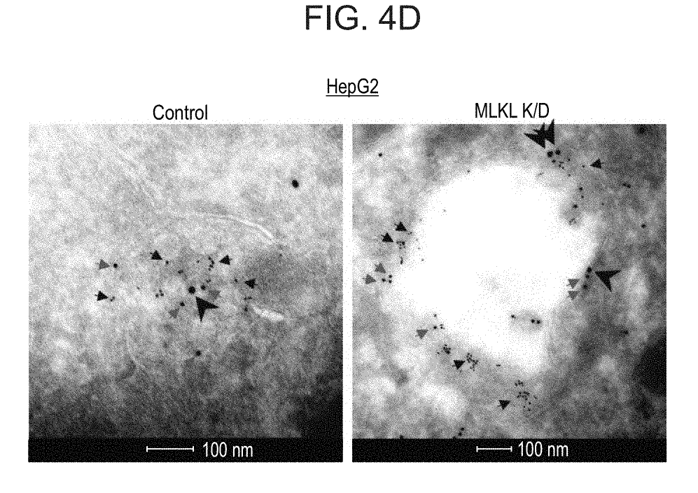

Electron microscopic analysis of cells in which the MVB were marked by uptake of albumin molecules conjugated to gold particles revealed a dramatic decrease in protein content and diminished content of ILVs in the MVBs of MLKL-deficient cells, along with an increase in diameter of the MVB -limiting membrane (FIGS. 4A-C). Whereas in MLKL-expressing cells EGFR molecules could be discerned within the MVB cavity, in MLKL-deficient cells these molecules were largely restricted to the region of the MVB-limiting membrane (FIG. 4D). This finding further confirmed that the low release of exosomes by MLKL deficient cells reflects arrest of their actual generation within the cell.

### Example 5

### MLKL Controls Transport to the Late Endosomes

Analyses of the mechanisms of uptake of cell surface receptors following ligand binding have yielded detailed information about the control of translocation of proteins into the MVB [Katzmann D J et al., Nature reviews Molecular cell biology (2002) 3: 893-905]. To further explore the mechanism by which MLKL affects exosomal generation, the impact of MLKL knockdown and of triggering of MLKL phosphorylation on the destiny of specific cell surface receptors following their ligand-dependent uptake was assessed.

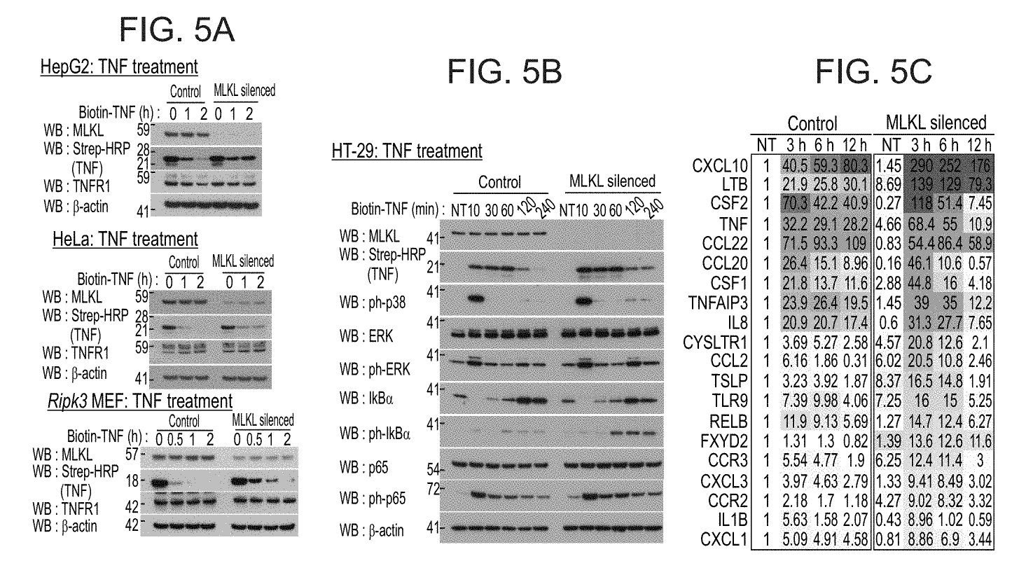

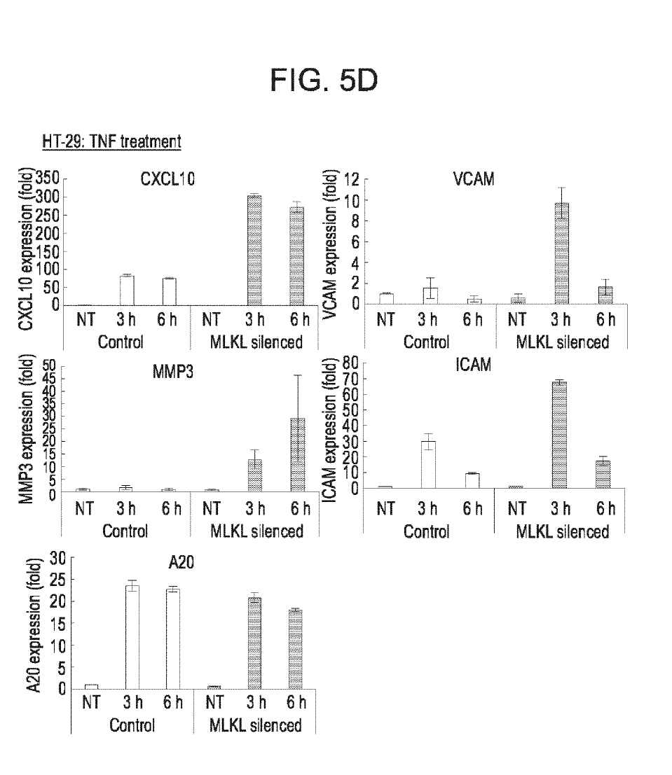

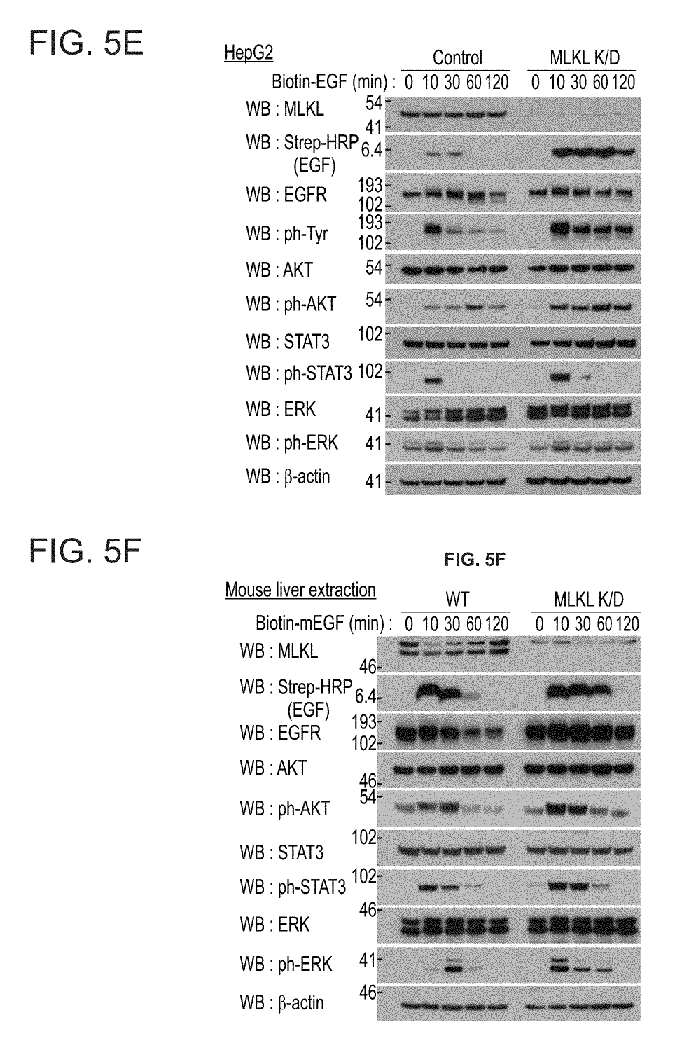

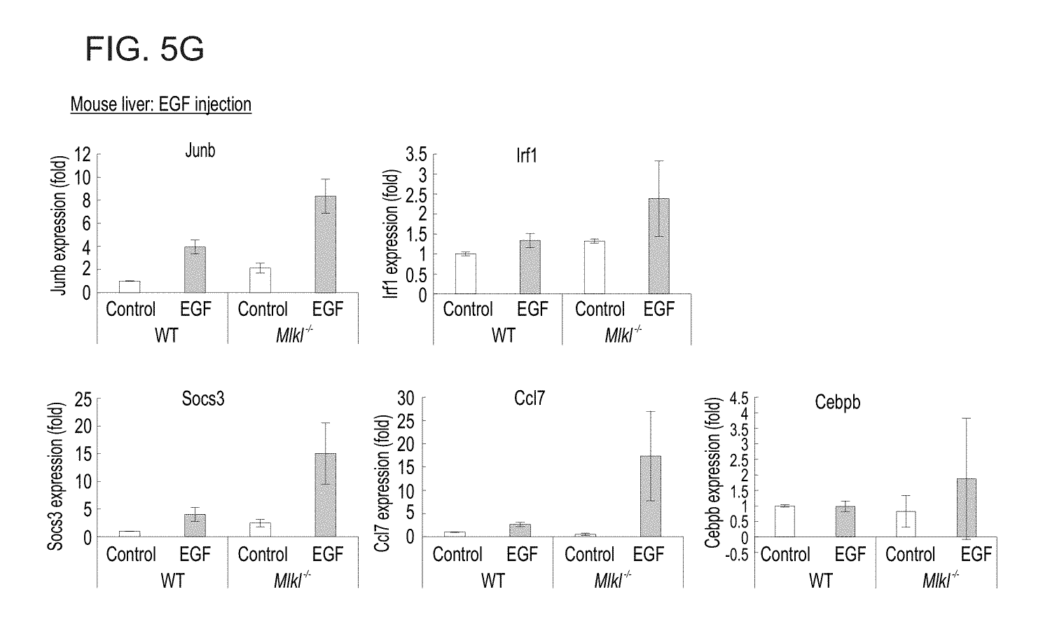

The present inventors analyzed the impact of MLKL deficiency on the cellular response to TNF. Besides their resistance to the necroptotic effect of this cytokine, cells deficient in MLKL also showed, unexpectedly, a marked reduction in the rate of intracellular degradation of TNF after its binding to the TNF receptor. This delay was observed both in RIPK3-expressing cell lines such as HT-29 and in RIPK3-deficient cells like HeLa and HepG2 and in MEFs in which Ripk3 was knocked out (FIG. 5A and data not shown), suggesting that it reflects a function of MLKL which is exerted independently of signaling for necroptosis.

The present inventors also examined the impact of MLKL deficiency on the cellular response to epidermal growth factor (EGF), a ligand of EGFR, a tyrosine kinase receptor. Like TNF, EGF is taken up and degraded, along with its receptor, in lysosomes. The present inventors found that knockdown of MLKL resulted in marked slowing down of the intracellular degradation of EGF and EGFR in vitro (FIG. 5E). Likewise, their in-vivo degradation in the livers of mice injected with EGF was slower in MLKL-knockout mice than in the wild type (FIG. 5F).

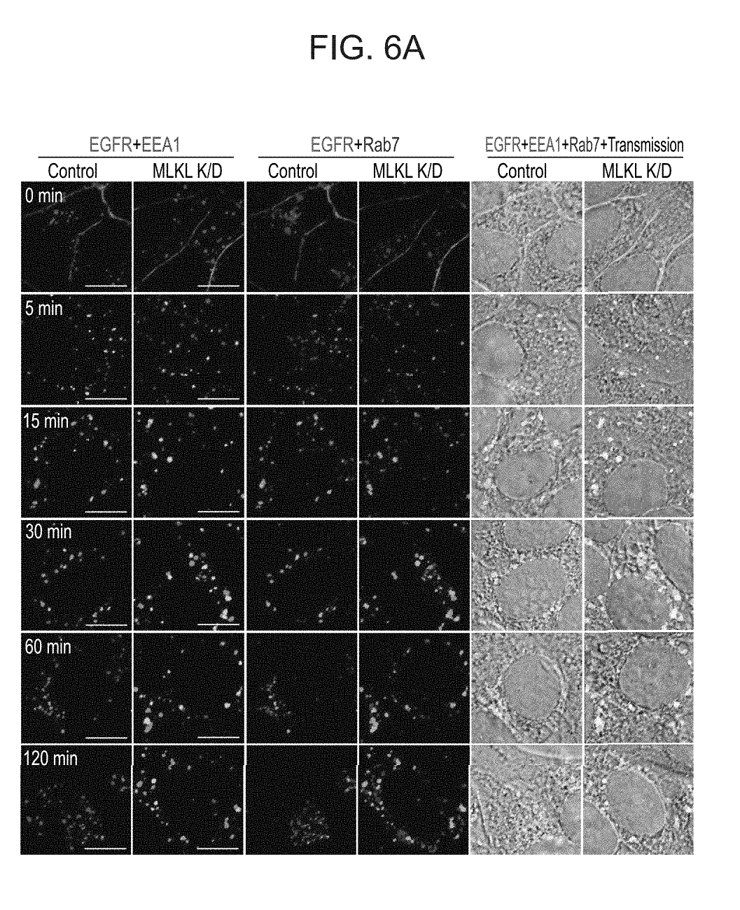

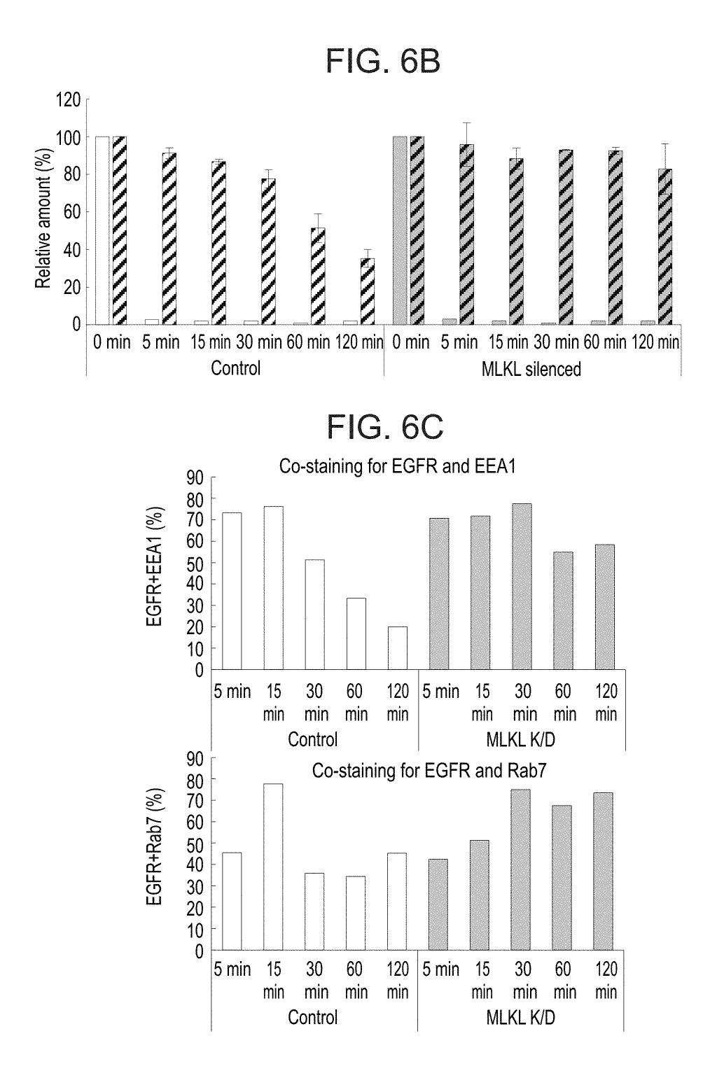

To define the intracellular site of this arrest, the HepG2 cells were co-immunostained with antibodies against the EGFR, with antibodies against EEA1, a marker of the early endosomes, and with antibodies against Rab7, a marker of the late endosomes, at different times after application of EGF to the cells. As shown in FIGS. 6A-C, the EGFR was taken up in both MLKL-expres sing and MLKL-deficient cells at about the same rate and transported to the early endosomes. However, further translocation of the receptor to the late endosomes and the pursuant degradation of the EGFR were delayed.

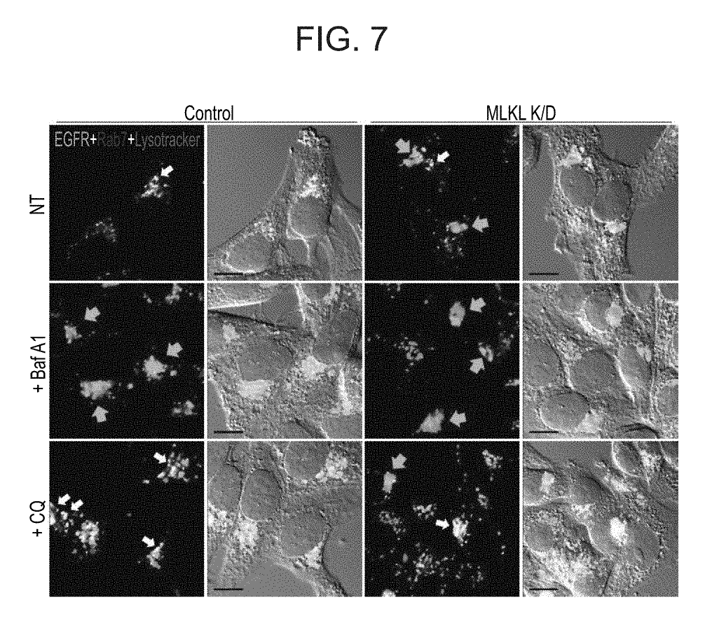

Assessing the impact of chloroquine, a lysosomal inhibitor, and of bafilomycin A1, an inhibitor of protein transport to the lysosomes, on the fate of the EGFR in the tested cells, both drugs were found to withhold the decrease in the cellular levels of the receptor and to obliterate the difference between cells expressing MLKL and cells deficient of it (FIG. 7).

Together, the findings indicated that MLKL affects trafficking at the stage of the late exosomes, assisting transport to the interior of the MVB and then further to the exosomes and to lysosomes.

### Example 6

### The Exosomes Released from Cells in which the Necrototic Pathway is Activated can Serve as Mediators of Inflammation

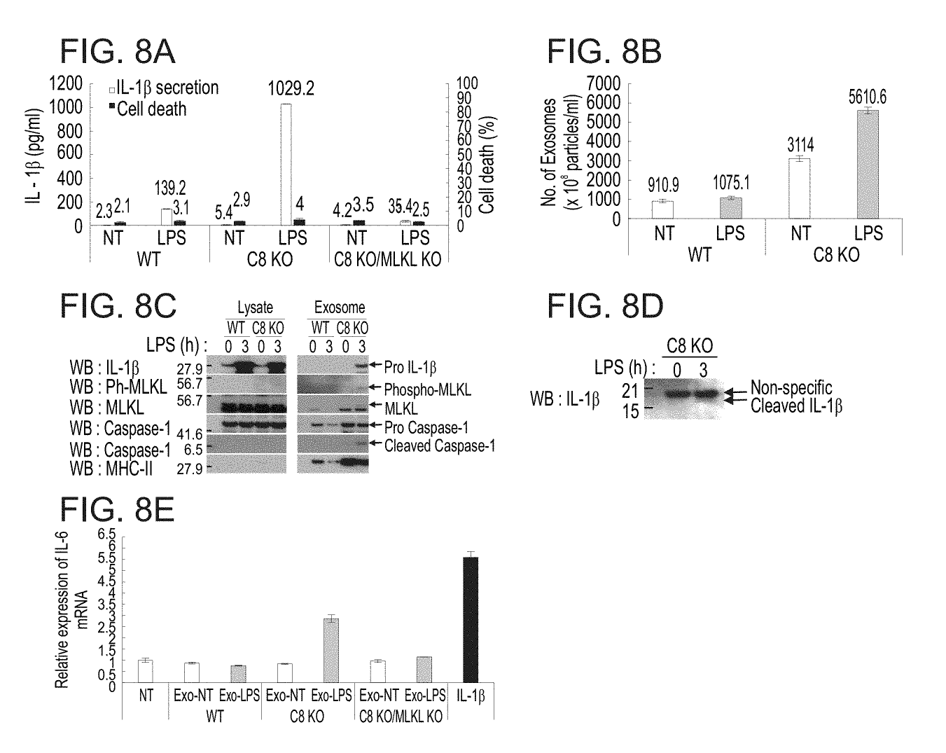

A prior study (Kang et al. (2013) supra) revealed that, besides triggering necrotic death, a process that prompts inflammation in association with tissue damage, activation of the necroptotic pathway can also trigger inflammation in other manners, independent of cell death (see FIG. 8A in which the yield of IL-1β in response to activation of the necroptotic pathway in dendritic cells is compared to the extent of death induction). Activation of the necroptotic pathway by LPS in bone marrow derived dendritic cells enhanced exosome release (FIG. 8B). Western blot analysis revealed that these exosomes contained phospho-MLKL as well IL-1β and the processed form of caspase-1- the enzyme activating IL-1β (FIGS. 8C-D). When applied to bone-marrow derived macrophages, these exosomes triggered expression of the gene encoding the inflammatory cytokine IL-6 (FIG. 8E).

### Example 7

### The Arrest of Endosomal Trafficking by Ablation of MLKL Function Potentiates Signaling by Cell Surface Ligands

Some of the signaling activities of receptors for extracellular ligands are maintained at their initial stages of their endosomal uptake. Some even occur only at that stage. However, all signaling activities of such receptors are ablated once these receptors and their ligands reach the lysosomes and are degraded therein. The discovery that MLKL function is required for effective endosomal uptake implies that arrest of this function, which results in slow down of such uptake, should also result in augmentation of some of the signaling activities of receptors that are taken up by cells. Indeed, as shown in FIG. 5B the slowdown of the uptake of the TNF receptors in MLKL deficient cells resulted in boosting of several TNF-induced signaling activities. It also resulted in upregulation of the induction of several genes by TNF (FIGS. 5C and D).

Likewise, the slowdown of uptake of the EGF receptor resulted in boosting of several EGF-induced signaling activities as well as in some increase in the basal level of the EGFR (FIGS. 5E and 5F) and in upregulation of the induction of several genes by EGF (FIG. 5G)

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of detecting activation of a necroptosis activation pathway in a subject, the method comprising:
(a) obtaining a biological sample comprising exosomes from the subject;
(b) purifying an exosome fraction of the biological sample;
(c) detecting an activity or expression of a component of the necroptosis activation pathway in an exosome fraction of the biological sample, wherein when an increase in said activity or expression of said component of said necroptosis activation pathway in said exosome fraction is beyond a predetermined threshold with respect to an activity or expression of said component of said necroptosis activation pathway in an exosome fraction from a non-necroptotic sample is indicated the sample is considered as having said activation of said necroptosis activation pathway.

2. A method of diagnosing a disease associated with activation of a necroptosis activation pathway in a subject, the method comprising:
(a) detecting activation of a necroptosis activation pathway in a biological sample of the subject according to claim 1; and
(b) diagnosing the subject as having said disease associated with said activation of said necroptosis activation pathway when an increase in said activity or expression of said component of said necroptosis activation pathway in said exosome fraction is beyond a predetermined threshold with respect to an activity or expression of said component of said necroptosis activation pathway in an exosome fraction from a non-necroptotic sample.

3. The method of claim 2, wherein said disease associated with said activation of said necroptosis activation pathway is selected from the group consisting of a necroptosis, an inflammation, a tissue damage, a tissue injury, a myocardinal infarction, a stroke, an ischemia-reperfusion injury (IRI), an atherosclerosis, a psoriasis, a pancreatitis, an inflammatory bowel disease, and a neurodegeneration.

4. A method of detecting necroptosis or inflammation in a subject, the method comprising:
(a) obtaining a biological sample comprising exosomes from the subject;
(b) purifying an exosome fraction of the biological sample;
(c) detecting a level of exosomes in said biological sample, wherein when an increase in said level is beyond a predetermined threshold with respect to a level of said exosomes in a biological sample from a non-necroptotic sample is indicated the sample is considered as a necroptotic or inflammatory sample.

5. A method of diagnosing necroptosis or inflammation in a subject, the method comprising:
(a) detecting a level of exosomes in a biological sample of the subject according to claim 4; and
(b) diagnosing the subject as having necroptosis or inflammation when an increase in said level of exosomes in said biological sample is beyond a predetermined threshold with respect to a level of said exosomes in a biological sample from a non-necroptotic sample.

6. (canceled)

7. The method of claim 4, further comprising measuring an activity or expression of a component of a necroptosis activation pathway in said exosomes, wherein a ratio of said activity or expression of said component of said necroptosis activation pathway per level of exsosomes beyond a predetermined threshold is indicative of necroptosis or inflammation.

8. A method of identifying a tissue undergoing necroptosis in a subject, the method comprising:
(a) obtaining a biological sample from the subject;
(b) purifying an exosome fraction of the biological sample;
(c) detecting an activity or expression of a component of a necroptosis activation pathway and an expression of a cell specific marker in an exosome fraction of the biological sample;
(d) identifying the tissue undergoing necroptosis based on the measured level of said activity or expression of said component of said necroptosis activation pathway and said expression of said cell specific marker.

9-11. (canceled)

12. The method of claim 8, wherein said exosomes co-express said component of said necroptosis activation pathway and said cell specific marker.

13. (canceled)

14. The method of claim 1, wherein the exosome fraction is essentially free of cells.

15-16. (canceled)

17. A method of treating a necroptosis or an inflammation in a subject in need thereof, the method comprising selecting a subject identified as having the necroptosis or the inflammation in accordance with the method of claim 5, and administering an anti-necroptosis therapy or an anti-inflammatory therapy to the subject.

18. (canceled)

19. The method of claim 17, wherein said necroptosis is associated with a disease selected from the group consisting of a tissue damage, a tissue injury, an inflammation, a myocardinal infarction, a stroke, an ischemia-reperfusion injury (IRI), an atherosclerosis, a psoriasis, a pancreatitis, an inflammatory bowel disease, and a neurodegeneration.

20. The method of claim 17, wherein said anti-necroptosis therapy comprises an anti-inflammatory agent, an immunosuppressant agent, non-steroid anti-inflammatory drugs (NSAIDs) or a small molecule inhibitor of necroptosis.

21. The method of claim 17, wherein said anti-necroptosis therapy comprises an agent for downregulating an activity or expression of at least one of MLKL, RIPK1, RIPK3, TNF-α or a Toll-like receptor ligand.

22. The method of claim 21, wherein said agent for downregulating said activity or expression of said MLKL specifically compromises necroptotic activity of said MLKL without compromising an endocytic activity of said MLKL.

23-24. (canceled)

25. The method of claim 5, wherein said inflammation is associated with a disease selected from the group consisting of an infectious disease, an autoimmune disease, a hypersensitivity associated inflammation, a graft rejection and an injury.

26-43. (canceled)

44. The method of claim 1, wherein said component of said necroptosis activation pathway comprises a mixed lineage kinase domain-like protein (MLKL).

45. The method of claim 44, wherein said MLKL comprises:
a phosphorylated MLKL; or
a constitutively active mutant; or
a phosphorylated MLKL comprising a phospho-mimetic mutation at an amino acid residue that is the target of phosphorylation by RIPK3; or
a phosphorylated MLKL comprising a phospho-mimetic mutation at an amino acid residue within the ATP-binding pocket of said MLKL.

46-48. (canceled)

49. The method of claim 1, wherein said component of said necroptosis activation pathway comprises a receptor interacting protein kinase 1 (RIPK1) or a receptor interacting protein kinase 3 (RIPK3).

50. The method, pharmaceutical composition or population of exosomes for use of claim 49, wherein said RIPK1 or RIPK3 comprises:
a phosphorylated RIPK1 or RIPK3; or
a constitutively active mutant.

51. (canceled)

52. The method of claim 1, wherein the exosomes have a particle size of about 20 to about 200 nm.

53-55. (canceled)


SEQUENCE LISTING (2 sequences)

SEQ ID NO: 1 - DNA, 23 bp, Artificial Sequence
  RNAi duplex for Rab27a
uauguuugucccauuggcagctt

SEQ ID NO: 2 - DNA, 23 bp, Artificial Sequence
  RNAi duplex for Rab27b
uacuguagugaugaauuugggtt